(19) United States
(12) Patent Application Publication
(10) Pub. No.: US 2024/0417378 A1
(43) Pub. Date: Dec. 19, 2024

(54) G-PROTEIN-COUPLED RECEPTOR REGULATORS AND METHODS OF USE THEREOF

(71) Applicant: Baylor College of Medicine, Houston, TX (US)

(72) Inventors: Damian Winston Young, Houston, TX (US); Srinivas Chamakuri, Houston, TX (US); Conrad Santini, Athens, GA (US); Martin M. Matzuk, Houston, TX (US); Kevin A. Tran, Houston, TX (US); Prashi Jain, Houston, TX (US); Shiva Krishna Reddy Guduru, Chapel Hill, NC (US); Idris O. Raji, Boston, MA (US); Errol L.G. Samuel, Houston, TX (US); Kevin R. MacKenzie, Houston, TX (US)

(73) Assignee: Baylor College of Medicine, Houston, TX (US)

(21) Appl. No.: 18/699,847

(22) PCT Filed: Oct. 11, 2022
(86) PCT No.: PCT/US2022/077907

§ 371 Date: Apr. 9, 2024

Related U.S. Application Data
(60) Provisional application No. 63/262,376, filed on Oct. 11, 2021.

(51) Int. Cl. C07D 243/08; A61K 31/495; A61K 45/06; C07D 243/10; C07D 265/30; C07D 487/04; C07D 487/10; C07D 498/04

(52) U.S. Cl. CPC C07D 243/08; A61K 31/495; A61K 45/06; C07D 243/10; C07D 265/30; C07D 487/04; C07D 487/10; C07D 498/04

(57) ABSTRACT

G protein-coupled receptor (GPCR) regulators and methods for their use are provided herein. The GPCR regulators described herein are useful in treating and/or preventing conditions or diseases associated with a GPCR, including ageing, cancer, cardiovascular disorders, hematologic disorders, infectious diseases, inflammatory diseases, metabolic diseases, neurodegenerative disorders, respiratory diseases, or urological disorders. Also provided are methods of regulating a G protein-coupled receptor in a cell using the compounds and compositions described herein.

### CROSS-REFERENCE TO PRIORITY APPLICATION

This application claims priority to U.S. Provisional Application No. 63/262,376, filed Oct. 11, 2021, which is incorporated herein by reference in its entirety.

### STATEMENT REGARDING FEDERALLY FUNDED RESEARCH

This invention was made with government support under Contract No. HHSN-271-2018-00023-C, awarded by the National Institute of Mental Health. The government has certain rights in the invention.

### BACKGROUND

G protein-coupled receptors (GPCRs) are proteins that exist within the cell membrane and transfer information encoded within neurotransmitters and drugs into cell responses. GPCRs are implicated in a host of conditions, diseases, and disorders. In fact, the GPCR family is considered as the largest group of cell surface receptors with the greatest potential of tunability, such that agents can be designed to target and regulate specific receptors. As such, GPCRs remain important drug targets.

### SUMMARY

Described herein are G protein-coupled receptor (GPCR) regulators and methods for their use. The GPCR regulators described herein are useful in treating and/or preventing conditions or diseases associated with a GPCR, including ageing, cancer, cardiovascular disorders, hematologic disorders, infectious diseases, inflammatory diseases, metabolic diseases, neurodegenerative disorders, respiratory diseases, or urological disorders, as further described herein.

GPCR regulators as described herein include compounds of the following formula:

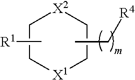

and pharmaceutically acceptable salts or prodrugs thereof. In these compounds, m is 0 or 1; X¹ is NR², ⁺NR²R², or O; X² is NR³, ⁺NR³R³, or O; R¹ is substituted or unsubstituted alkyl, substituted or unsubstituted heteroalkyl, substituted or unsubstituted aryl, substituted or unsubstituted heteroaryl; each R² and R³ are independently selected from the group consisting of hydrogen, substituted or unsubstituted alkyl, substituted or unsubstituted carbonyl, substituted or unsubstituted sulfonyl, substituted or unsubstituted aryl, or substituted or unsubstituted heteroaryl; and R⁴ is substituted or unsubstituted alkyl, substituted or unsubstituted amino, hydroxyl, substituted or unsubstituted alkoxy, substituted or unsubstituted carbonyl, or substituted or unsubstituted sulfonyl. In these compounds, X¹ and X² are not simultaneously O; and two of R¹, R², R³, and R⁴, when present and adjacent, can combine to form a substituted or unsubstituted heterocycloalkyl or a substituted or unsubstituted heteroaryl.

In some examples, GPCR regulators as described herein include compounds of the following formula:

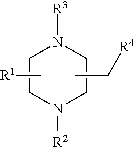

and pharmaceutically acceptable salts or prodrugs thereof, wherein R¹ is substituted or unsubstituted alkyl, substituted or unsubstituted heteroalkyl, substituted or unsubstituted aryl, substituted or unsubstituted heteroaryl; R² and R³ are each independently selected from the group consisting of hydrogen, substituted or unsubstituted alkyl, substituted or unsubstituted carbonyl, substituted or unsubstituted sulfonyl, substituted or unsubstituted aryl, or substituted or unsubstituted heteroaryl; and R⁴ is substituted or unsubstituted alkyl, substituted or unsubstituted amino, hydroxyl, substituted or unsubstituted alkoxy, substituted or unsubstituted carbonyl, or substituted or unsubstituted sulfonyl, wherein R¹ and R³, R¹ and R², R² and R⁴, or R³ and R⁴ can combine to form a substituted or unsubstituted heterocycloalkyl or a substituted or unsubstituted heteroaryl. Optionally, R¹ is a side chain of an amino acid (e.g., a natural amino acid or an unnatural amino acid). Optionally, the amino acid is a natural amino acid selected from the group consisting of alanine, valine, isoleucine, leucine, methionine, phenylalanine, tyrosine, tryptophan, serine, threonine, asparagine, glutamine, cysteine, glycine, proline, arginine, histidine, lysine, aspartic acid, and glutamic acid. In some cases, R¹ is methyl.

Optionally, R² and R³ are each independently selected from the group consisting of hydrogen, methyl, acetyl, —SO₂CH₃, phenyl, benzyl, benzoyl, aryl-substituted sulfonyl, heteroaryl-substituted sulfonyl, formyl, —CO₂R⁶, and —CONHR⁶, wherein R⁶ is hydrogen, substituted or unsubstituted alkyl, substituted or unsubstituted heteroalkyl, substituted or unsubstituted aryl, or substituted or unsubstituted heteroaryl. Optionally, R⁴ is —CO₂R⁵, wherein R⁵ is hydrogen, substituted or unsubstituted alkyl, substituted or unsubstituted heteroalkyl, substituted or unsubstituted aryl, or substituted or unsubstituted heteroaryl. In some cases, R⁴ is —CO₂H.

Optionally, the compound has one of the following formulas:

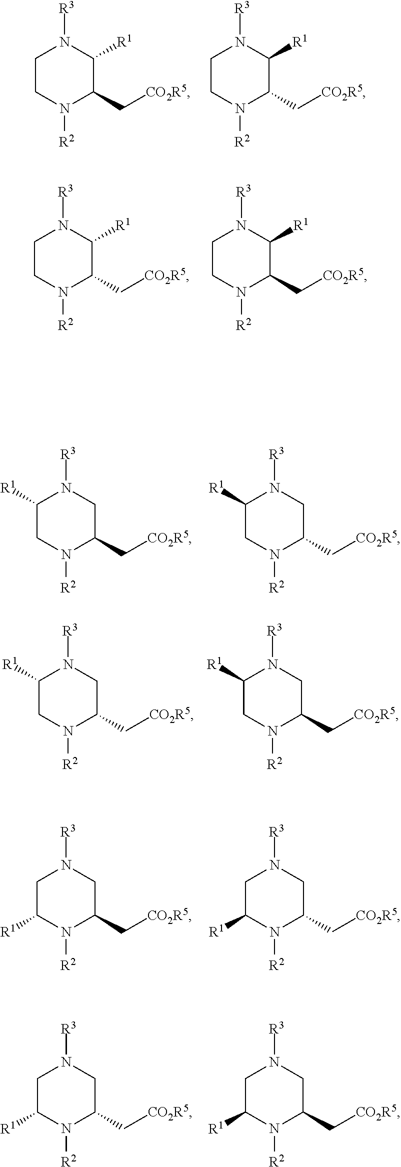

wherein R⁵ is hydrogen, substituted or unsubstituted alkyl, substituted or unsubstituted heteroalkyl, substituted or unsubstituted aryl, or substituted or unsubstituted heteroaryl.

Optionally, the compound has the following formula:

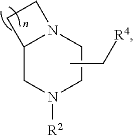

wherein n is 0, 1, 2, or 3.

Optionally, the compound has the following formula:

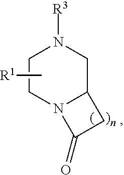

wherein n is 1, 2, or 3.

Optionally, the compound has one of the following formulas:

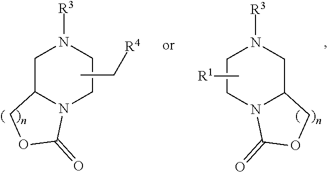

wherein n is 1 or 2.

In some examples, GPCR regulators as described herein include compounds of the following formula:

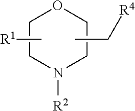

and pharmaceutically acceptable salts or prodrugs thereof, wherein R¹ is substituted or unsubstituted alkyl, substituted or unsubstituted heteroalkyl, substituted or unsubstituted aryl, substituted or unsubstituted heteroaryl; R² is hydrogen, substituted or unsubstituted alkyl, substituted or unsubstituted carbonyl, substituted or unsubstituted sulfonyl, substituted or unsubstituted aryl, or substituted or unsubstituted heteroaryl; and R⁴ is substituted or unsubstituted alkyl, substituted or unsubstituted amino, hydroxyl, substituted or unsubstituted alkoxy, substituted or unsubstituted carbonyl, or substituted or unsubstituted sulfonyl. In these compounds, R¹ and R² or R² and R⁴ can optionally combine to form a substituted or unsubstituted heterocycloalkyl or a substituted or unsubstituted heteroaryl.

In some examples, GPCR regulators as described herein include compounds of the following formula:

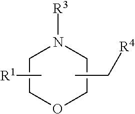

and pharmaceutically acceptable salts or prodrugs thereof, wherein R¹ is substituted or unsubstituted alkyl, substituted or unsubstituted heteroalkyl, substituted or unsubstituted aryl, substituted or unsubstituted heteroaryl; R³ is hydrogen, substituted or unsubstituted alkyl, substituted or unsubstituted carbonyl, substituted or unsubstituted sulfonyl, substituted or unsubstituted aryl, or substituted or unsubstituted heteroaryl; and R⁴ is substituted or unsubstituted alkyl, substituted or unsubstituted amino, hydroxyl, substituted or unsubstituted alkoxy, substituted or unsubstituted carbonyl, or substituted or unsubstituted sulfonyl. Optionally, R¹ and R³ or R³ and R⁴ can combine to form a substituted or unsubstituted heterocycloalkyl or a substituted or unsubstituted heteroaryl.

In some examples, the GPCR regulators as described herein include compounds of the following formula:

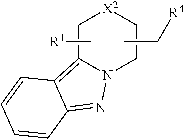

and pharmaceutically acceptable salts or prodrugs thereof, wherein X² is NR² or O; R¹ is substituted or unsubstituted alkyl, substituted or unsubstituted heteroalkyl, substituted or unsubstituted aryl, or substituted or unsubstituted heteroaryl; and R⁴ is substituted or unsubstituted alkyl, substituted or unsubstituted amino, hydroxyl, substituted or unsubstituted alkoxy, substituted or unsubstituted carbonyl, or substituted or unsubstituted sulfonyl. Optionally, the compound has the following formula:

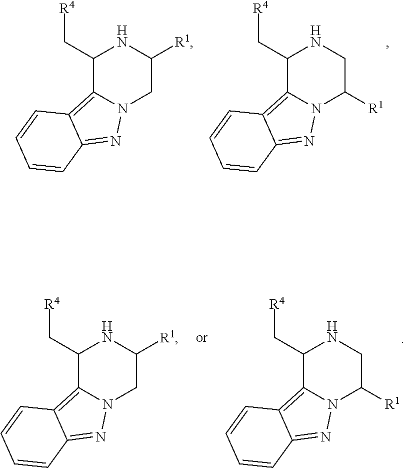

In some examples, GPCR regulators as described herein include compounds of the following formula:

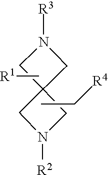

and pharmaceutically acceptable salts or prodrugs thereof, wherein R¹ is substituted or unsubstituted alkyl, substituted or unsubstituted heteroalkyl, substituted or unsubstituted aryl, or substituted or unsubstituted heteroaryl; R² and R³ are independently selected from the group consisting of hydrogen, substituted or unsubstituted alkyl, substituted or unsubstituted carbonyl, substituted or unsubstituted sulfonyl, substituted or unsubstituted aryl, or substituted or unsubstituted heteroaryl; and R⁴ is substituted or unsubstituted alkyl, substituted or unsubstituted amino, hydroxyl, substituted or unsubstituted alkoxy, substituted or unsubstituted carbonyl, or substituted or unsubstituted sulfonyl.

Also described herein are pharmaceutical compositions comprising a compound as described herein and a pharmaceutically acceptable carrier.

Further described herein is a kit comprising a compound or a pharmaceutical composition as described herein.

Methods of treating and/or preventing a condition or disease associated with a G protein-coupled receptor in a subject are also provided herein. A method of treating or preventing a condition or disease associated with a G protein-coupled receptor in a subject comprises administering to the subject an effective amount of a compound or a pharmaceutical composition as described herein. Optionally, the condition or disease associated with a G protein-coupled receptor comprises ageing, cancer, a cardiovascular disorder, a hematologic disorder, an infectious disease, an inflammatory disease, a metabolic disease, a neurodegenerative disorder, a respiratory disease, or a urological disorder. In some cases, the method can further comprise administering to the subject a second compound, biomolecule, or composition.

Methods of regulating a G protein-coupled receptor in a cell are also provided herein. A method of regulating a G protein-coupled receptor comprises contacting a cell with an effective amount of a compound as described herein. Optionally, the G protein-coupled receptor is a serotonin receptor (e.g., 5-HT1A, 5-HT1B, 5-HT1D, 5-HT1E, 5-HT2A, 5-HT2B, 5-HT2C, 5-HT3, 5-HT5A, 5-HT6, and 5-HT7A), a dopamine receptor (e.g., D1, D2, D3, D4, and D5), a muscarinic receptor (e.g., M1, M2, M3, M4, and M5), an adrenergic receptor (e.g., Alpha-1A, Alpha-1B, Alpha-1C, Alpha-2A, Alpha-2B, Alpha-2C, Beta-1, Beta-2, and Beta-3), a histamine receptor (e.g., H1, H2, H3, and H4), or a sigma receptor (e.g., Sigma-1 and Sigma-2). Optionally, the G protein-coupled receptor is selected from the group consisting of kappa-opioid receptor (KOR), mu-opioid receptor (MOR), delta-opioid receptor (DOR), gamma-aminobutyric acid A (GABAA) receptor, peripheral-type benzodiazepine receptors (PBR), serotonin transporter (SERT) receptor, and benzodiazepine (BZP) rat brain receptors. The contacting can be performed in vitro or in vivo.

The details of one or more embodiments are set forth in the drawings and the description below. Other features, objects, and advantages will be apparent from the description and drawings, and from the claims.

### DESCRIPTION OF THE DRAWINGS

FIG. 1 is a heat map of a primary binding assay showing the mean % inhibition of compounds as described herein against G protein-coupled receptors (GPCRs).

FIG. 2 is a graph showing the results of a secondary binding assay of the indicated compound against GPCR H1. Chlorpheniramine maleate was used as the control.

FIG. 3 is a graph showing the results of a secondary binding assay of the indicated compound against GPCR KOR. Salvinorin A was used as the control.

FIG. 4 is a graph showing the results of a secondary binding assay of the indicated compound against GPCR DAT. GBR 12909 (also known as vanoxerine), a known dopamine uptake inhibitor, was used as the control.

FIG. 5 is a graph showing the results of a secondary binding assay of the indicated compound against GPCR Sigma 1. Haloperidol was used as the control.

FIG. 6 is a graph showing the results of a secondary binding assay of the indicated compound against GPCR Sigma 1. Haloperidol was used as the control.

FIG. 7 is a graph showing the results of a secondary binding assay of the indicated compound against GPCR KOR. Salvinorin A was used as the control.

FIG. 8 is a graph showing the results of a secondary binding assay of the indicated compound against GPCR 5-HT1D. Ergotamine tartrate was used as the control.

FIG. 9 is a graph showing the results of a secondary binding assay of the indicated compound against GPCR D3. Nemonapride was used as the control.

FIG. 10 is a graph showing the results of a secondary binding assay of the indicated compound against GPCR 5-HT2A. Clozapine was used as the control.

FIG. 11 is a graph showing the results of a secondary binding assay of the indicated compound against GPCR MOR. Morphine was used as the control.

FIG. 12 is a graph showing the results of a secondary binding assay of the indicated compound against GPCR Sigma 1. Haloperidol was used as the control.

FIG. 13 is a graph showing the results of a secondary binding assay of the indicated compound against GPCR 5-HT2B. SB 206553, a known selective 5-HT2B and 5-HT2C receptor antagonist, was used as the control.

FIG. 14 is a graph showing the results of a secondary binding assay of the indicated compound against GPCR MOR. Morphine was used as the control.

FIG. 15 is a graph showing the results of a secondary binding assay of the indicated compound against GPCR Sigma 1. Haloperidol was used as the control.

FIG. 16 is a graph showing the results of a secondary binding assay of the indicated compound against GPCR Sigma 1. Haloperidol was used as the control.

FIG. 17 is a graph showing the results of a secondary binding assay of the indicated compound against GPCR Sigma 1. Haloperidol was used as the control.

FIG. 18 is a graph showing the results of a secondary binding assay of the indicated compound against GPCR Sigma 1. Haloperidol was used as the control.

FIG. 19 is a graph showing the results of a secondary binding assay of the indicated compound against GPCR Sigma 1. Haloperidol was used as the control.

FIG. 20 is a graph showing the results of a secondary binding assay of the indicated compound against GPCR Sigma 1. Haloperidol was used as the control.

FIG. 21 is a graph showing the results of a secondary binding assay of the indicated compound against GPCR Sigma 1. Haloperidol was used as the control.

FIG. 22 is a graph showing the results of a secondary binding assay of the indicated compound against GPCR Sigma 1. Haloperidol was used as the control.

FIG. 23 is a graph showing the results of a secondary binding assay of the indicated compound against GPCR Sigma 1. Haloperidol was used as the control.

FIG. 24 is a graph showing the results of a secondary binding assay of the indicated compound against GPCR Sigma 1. Haloperidol was used as the control.

FIG. 25 is a graph showing the results of a secondary binding assay of the indicated compound against GPCR Sigma 1. Haloperidol was used as the control.

FIG. 26 is a graph showing the results of a secondary binding assay of the indicated compound against GPCR Sigma 1. Haloperidol was used as the control.

FIG. 27 is a graph showing the results of a secondary binding assay of the indicated compound against GPCR 5-HT1D. Ergotamine tartrate was used as the control.

FIG. 28 is a graph showing the results of a secondary binding assay of the indicated compound against GPCR BZP rat brain site. Clonazepam was used as the control.

FIG. 29 is a graph showing the results of a secondary binding assay of the indicated compound against GPCR KOR. Salvinorin A was used as the control.

FIG. 30 is a graph showing the results of a secondary binding assay of the indicated compound against GPCR Sigma 1. Haloperidol was used as the control.

FIG. 31A and FIG. 31B contain heat maps of a primary binding assay (FIG. 31A) and second binding assay (FIG. 31B), showing the mean % inhibition and pKi, respectively, of compounds as described herein against G protein-coupled receptors (GPCRs).

FIG. 32 is a chord plot showing the activity of compounds as described herein against G protein-coupled receptors (GPCR) in a dose-response screen.

### DETAILED DESCRIPTION

Described herein are G protein-coupled receptor (GPCR) regulators and methods for their use. The GPCR regulators described herein are useful in treating and/or preventing conditions or diseases associated with a GPCR, including ageing, cancer, cardiovascular disorders, hematologic disorders, infectious diseases, inflammatory diseases, metabolic diseases, neurodegenerative disorders, respiratory diseases, or urological disorders, as further described herein. The GPCR regulators described herein include heterocyclic compounds, such as piperazines and morpholines. The GPCR regulators described herein are designed with specific stereochemical configurations to target specific members within the GPCR family. As such, the compounds described herein exhibit unique, selective interactions with GPCRs.

### I. Compounds

A class of GPCR regulators described herein is represented by Formula I.

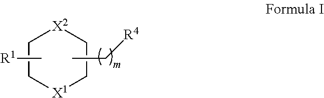

and pharmaceutically acceptable salts or prodrugs thereof.

In Formula I, m is 0 or 1.

Also in Formula I, X¹ is NR², ⁺NR²R², or O.

Additionally in Formula I, X² is NR³, ⁺NR³R³, or O.

Further in Formula I, R¹ is substituted or unsubstituted alkyl, substituted or unsubstituted heteroalkyl, substituted or unsubstituted aryl, substituted or unsubstituted heteroaryl.

Also in Formula I, each recitation of R² and R³ are independently selected from the group consisting of hydrogen, substituted or unsubstituted alkyl, substituted or unsubstituted carbonyl, substituted or unsubstituted sulfonyl, substituted or unsubstituted aryl, or substituted or unsubstituted heteroaryl. For example, when Formula I includes a quaternary amine (i.e., when X¹ is ⁺NR²R² or when X² is ⁺NR³R³, the R² groups or R³ groups, respectively, can each be the same substituent or a different substituent).

Additionally in Formula I, R⁴ is substituted or unsubstituted alkyl, substituted or unsubstituted amino, hydroxyl, substituted or unsubstituted alkoxy, substituted or unsubstituted carbonyl, or substituted or unsubstituted sulfonyl.

In Formula I, X¹ and X² are not simultaneously O.

Optionally, adjacent R groups in Formula I, e.g., R¹, R², R³, and R⁴, can be combined to form a substituted or unsubstituted heterocycloalkyl or a substituted or unsubstituted heteroaryl. For example, two of R¹, R², R³, and R⁴, when present and adjacent, can combine to form a substituted or unsubstituted heterocycloalkyl or a substituted or unsubstituted heteroaryl.

In some cases, the compounds according to Formula I are represented by Structure I-A:

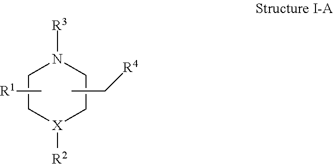

and pharmaceutically acceptable salts or prodrugs thereof.

In Structure I-A, R¹ is substituted or unsubstituted alkyl, substituted or unsubstituted heteroalkyl, substituted or unsubstituted aryl, substituted or unsubstituted heteroaryl. Optionally, R¹ is a side chain of an amino acid (e.g., a natural amino acid or an unnatural amino acid). Optionally, the amino acid is a natural amino acid selected from the group consisting of alanine, valine, isoleucine, leucine, methionine, phenylalanine, tyrosine, tryptophan, serine, threonine, asparagine, glutamine, cysteine, glycine, proline, arginine, histidine, lysine, aspartic acid, and glutamic acid. In some examples, R¹ is methyl. In some examples, R¹ is aryl-substituted alkyl or heteroaryl substituted alkyl. For example, R¹ can optionally be a methylene group substituted by a substituted or substituted pyridine, a methylene group substituted by a substituted or substituted phenyl (e.g., a halogen-substituted benzyl), or a methylene group substituted by a naphthyl. By way of non-limiting example, the compound described herein can have a structure as shown below:

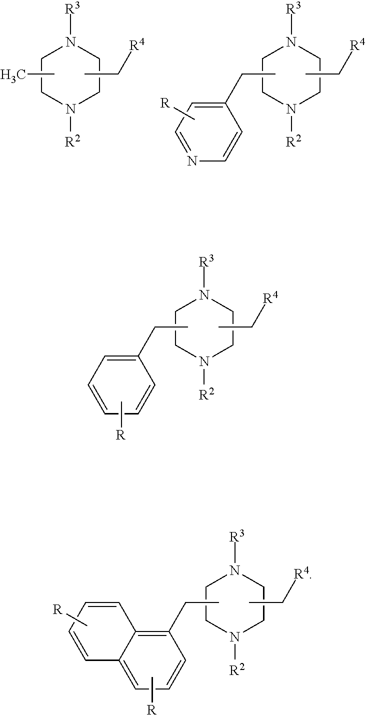

In the structures above, R can be hydrogen, hydroxyl, halogen, carboxy, alkoxy, cycloalkoxy, aryloxy, amino, alkyl, alkenyl, alkynyl, aryl, heteroalkyl, heteroalkenyl, heteroalkynyl, heteroaryl, cycloalkyl, or heterocycloalkyl.

Also in Structure I-A, R² and R³ are each independently selected from the group consisting of hydrogen, substituted or unsubstituted alkyl, substituted or unsubstituted carbonyl, substituted or unsubstituted sulfonyl, substituted or unsubstituted aryl, or substituted or unsubstituted heteroaryl. In some examples, R² and R³ are each independently selected from the group consisting of hydrogen, methyl, acetyl, —SO₂CH₃, phenyl, benzyl, benzoyl, aryl-substituted sulfonyl, heteroaryl-substituted sulfonyl, formyl, —CO₂R⁶, and —CONHR⁶, wherein R⁶ is hydrogen, substituted or unsubstituted alkyl, substituted or unsubstituted heteroalkyl, substituted or unsubstituted aryl, or substituted or unsubstituted heteroaryl. By way of non-limiting example, the compound described herein can have a structure as shown below:

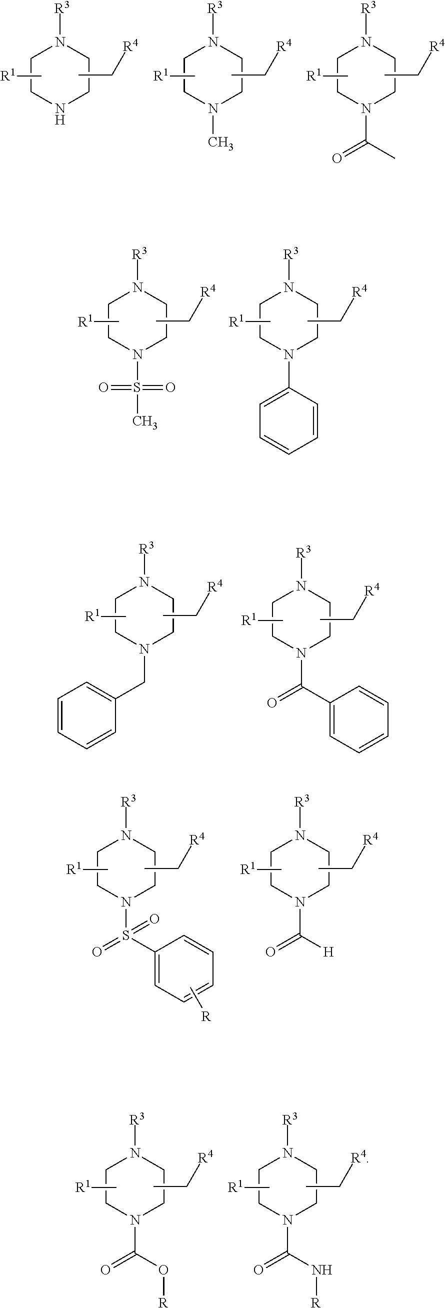

While the examples above depict options for R², the options shown for R² are also representative of options for R³. In the structures above, R can be hydrogen, hydroxyl, halogen, carboxy, alkoxy, cycloalkoxy, aryloxy, amino, alkyl, alkenyl, alkynyl, aryl, heteroalkyl, heteroalkenyl, heteroalkynyl, heteroaryl, cycloalkyl, or heterocycloalkyl.

Additionally in Structure I-A, R⁴ is substituted or unsubstituted alkyl, substituted or unsubstituted amino, hydroxyl, substituted or unsubstituted alkoxy, substituted or unsubstituted carbonyl, or substituted or unsubstituted sulfonyl. In some examples, R⁴ is —CO₂R⁵, wherein R⁵ is hydrogen, substituted or unsubstituted alkyl, substituted or unsubstituted heteroalkyl, substituted or unsubstituted aryl, or substituted or unsubstituted heteroaryl. Optionally, R⁴ is —CO₂H. By way of non-limiting example, the compound described herein can have a structure as shown below:

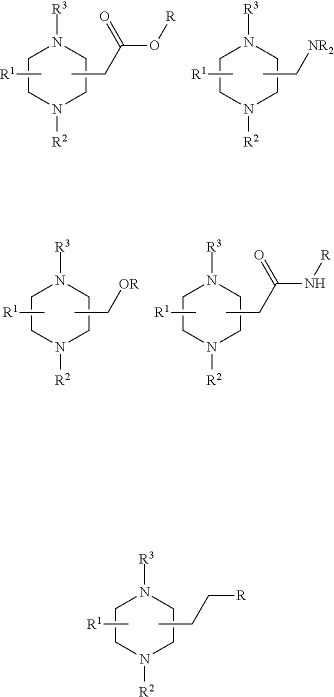

In the structures above, R can be hydrogen, hydroxyl, halogen, carboxy, alkoxy, cycloalkoxy, aryloxy, amino, alkyl, alkenyl, alkynyl, aryl, heteroalkyl, heteroalkenyl, heteroalkynyl, heteroaryl, cycloalkyl, or heterocycloalkyl.

Optionally in Structure I-A, R¹ and R³, R¹ and R², R² and R⁴, or R³ and R⁴ can combine to form a substituted or unsubstituted heterocycloalkyl or a substituted or unsubstituted heteroaryl. By way of non-limiting example, the compound described herein can have a structure according to Structure I-A13 as shown further below, wherein R¹ and R³ combine to form a substituted or unsubstituted heterocycloalkyl or a substituted or unsubstituted heteroaryl. By way of non-limiting example, the compound described herein can have a structure according to Structure I-A13 or Structure I-A14, as shown further below, wherein R² and R⁴ combine to form a substituted or unsubstituted heterocycloalkyl or a substituted or unsubstituted heteroaryl.

In some cases, the compounds according to Structure I-A are represented by Structure I-A1:

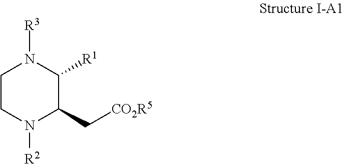

In Structure I-A1, R¹, R², and R³ are as defined above for Formula I. Also in Structure I-A1, R⁵ is hydrogen, substituted or unsubstituted alkyl, substituted or unsubstituted heteroalkyl, substituted or unsubstituted aryl, or substituted or unsubstituted heteroaryl.

Examples of Structure I-A1 include the following compounds:

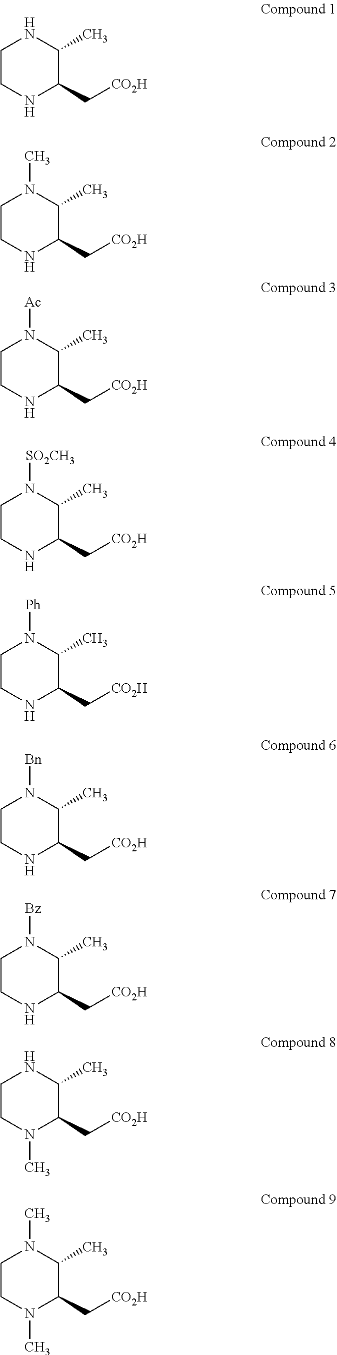

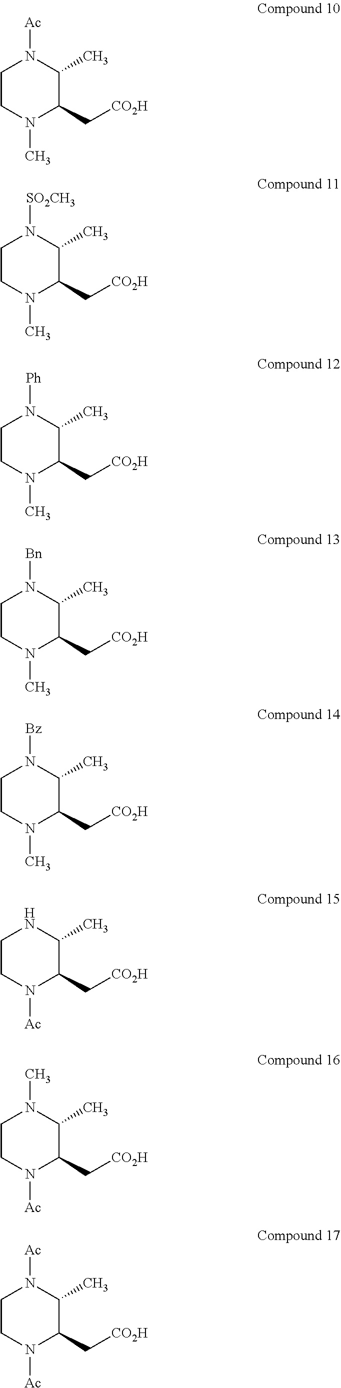

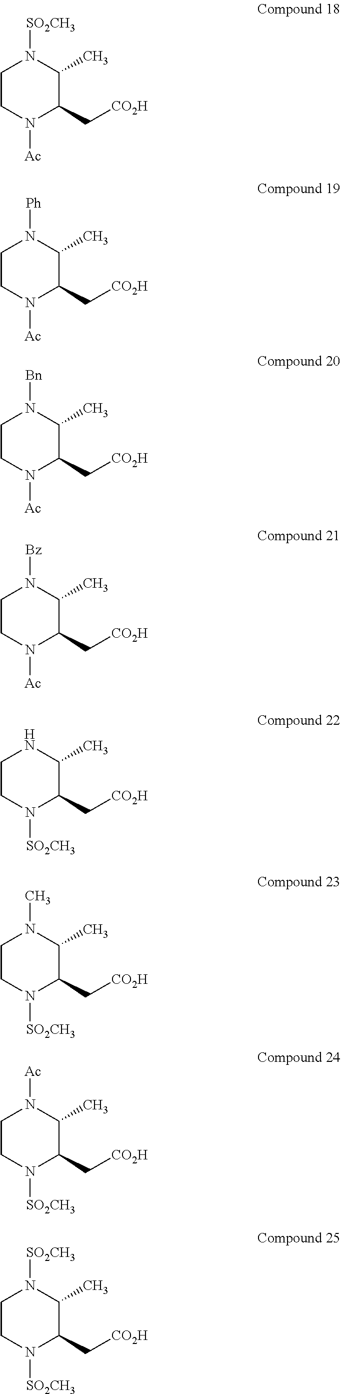

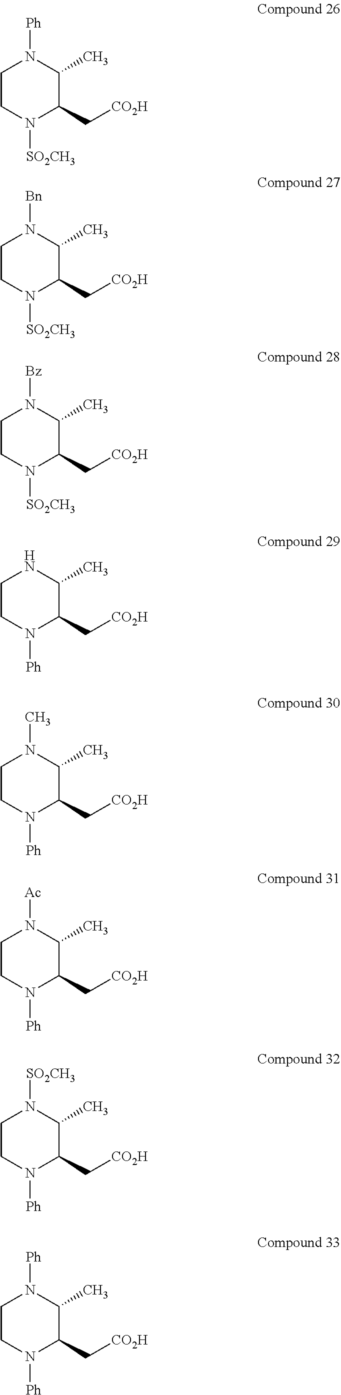

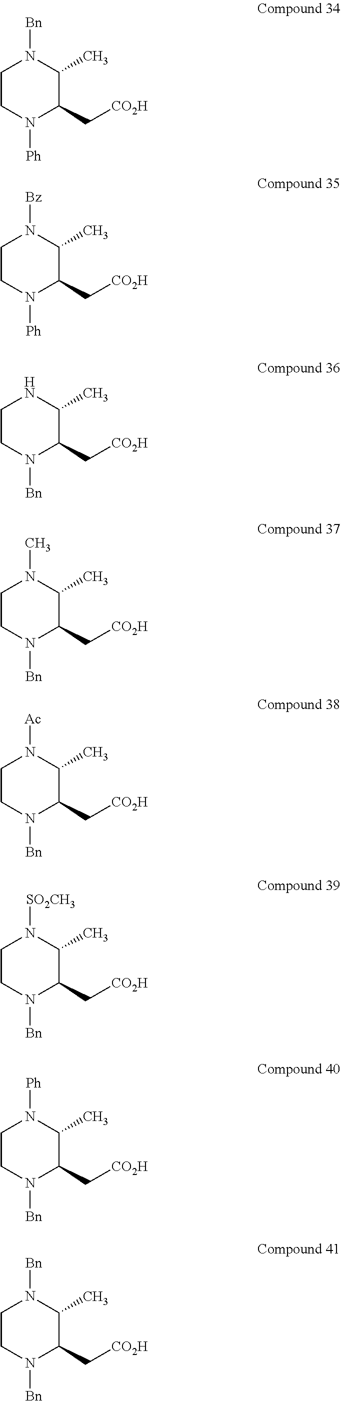

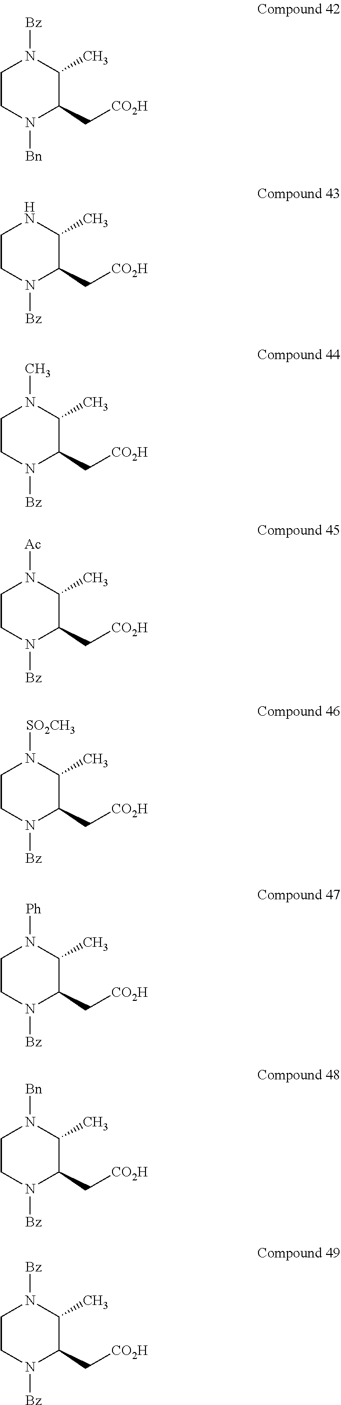

In some cases, the compounds according to Structure I-A are represented by Structure I-A2:

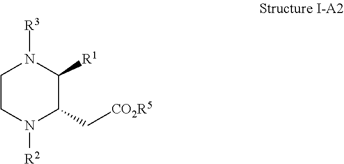

In Structure I-A2, R¹, R², and R³ are as defined above for Formula I. Also in Structure I-A2, R⁵ is hydrogen, substituted or unsubstituted alkyl, substituted or unsubstituted heteroalkyl, substituted or unsubstituted aryl, or substituted or unsubstituted heteroaryl.

Examples of Structure I-A2 include the following compounds:

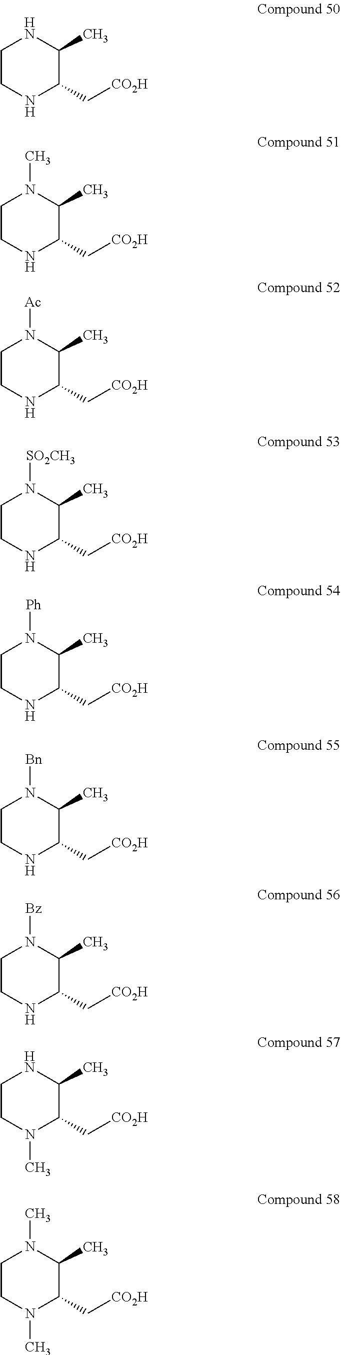

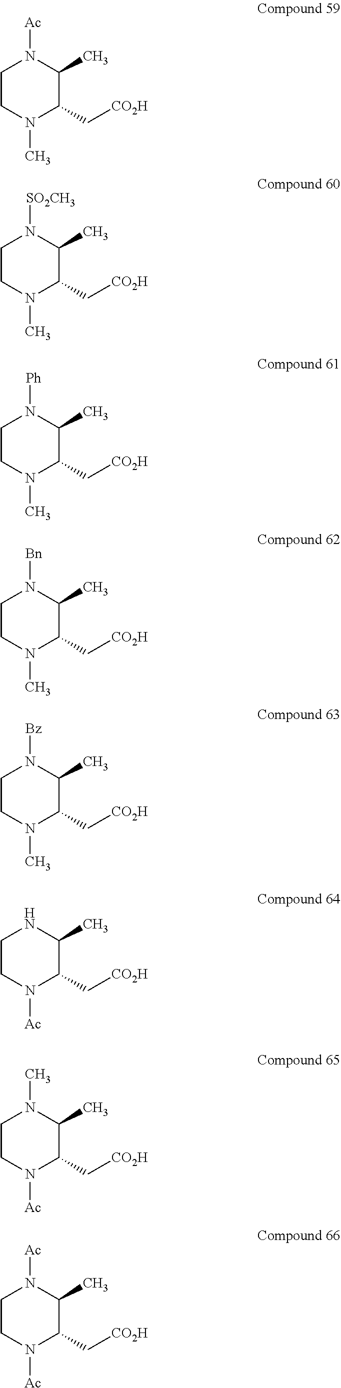

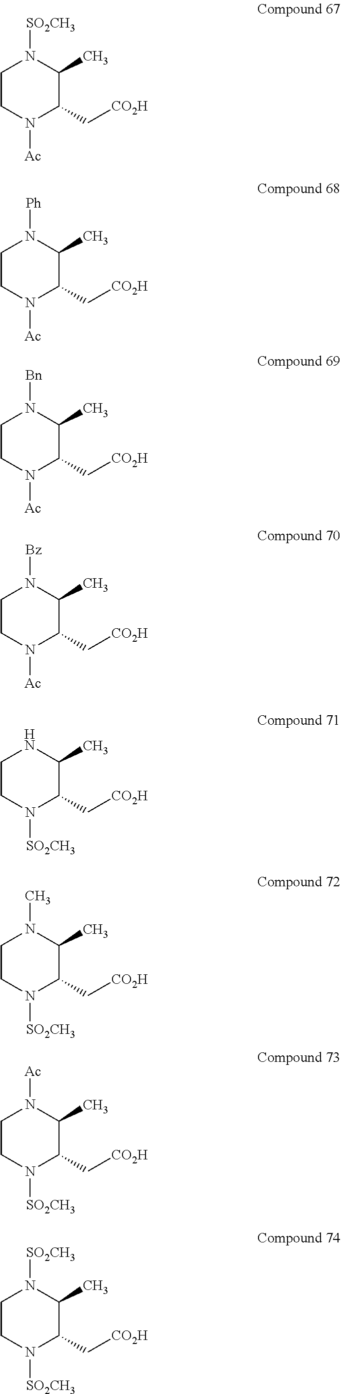

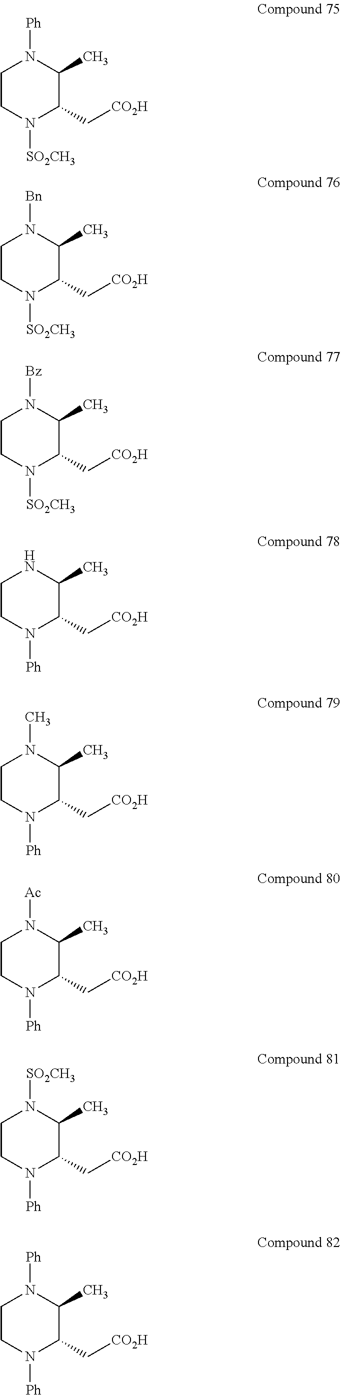

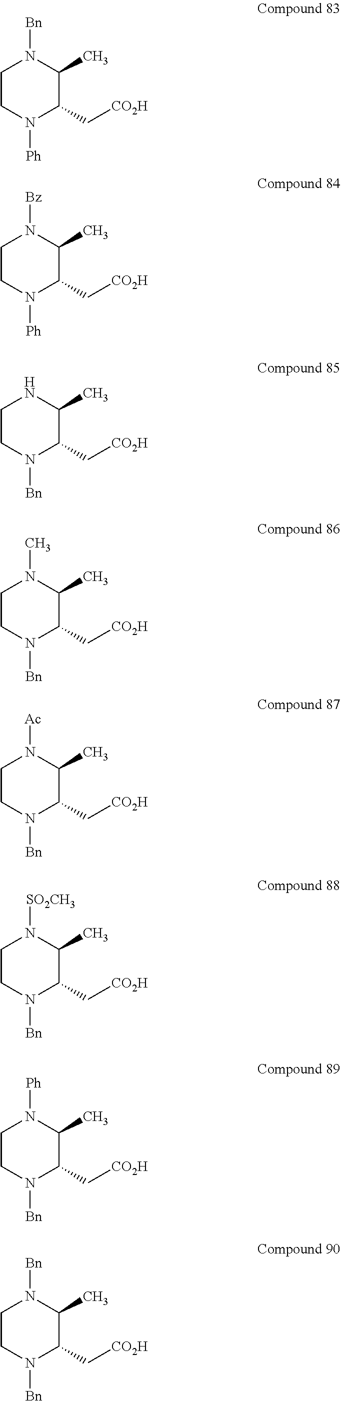

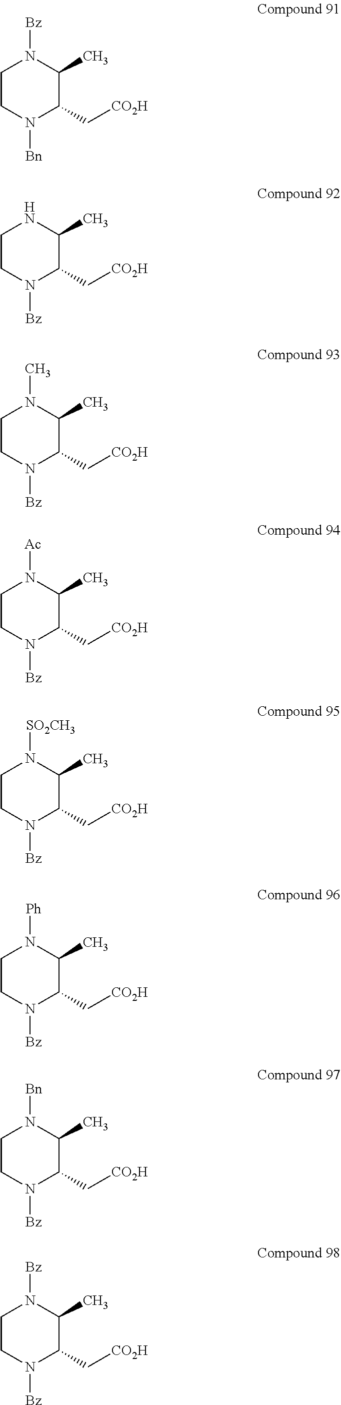

In some cases, the compounds according to Structure I-A are represented by Structure I-A3:

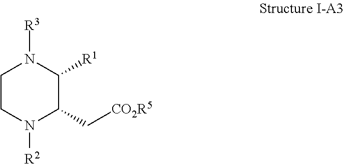

In Structure I-A3, R¹, R², and R³ are as defined above for Formula I. Also in Structure I-A3, R⁵ is hydrogen, substituted or unsubstituted alkyl, substituted or unsubstituted heteroalkyl, substituted or unsubstituted aryl, or substituted or unsubstituted heteroaryl.

Examples of Structure I-A3 include the following compounds:

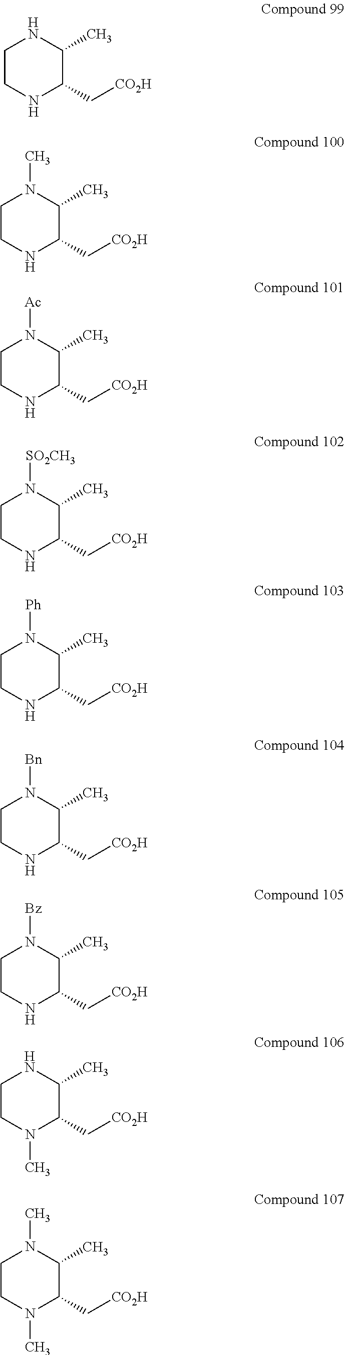

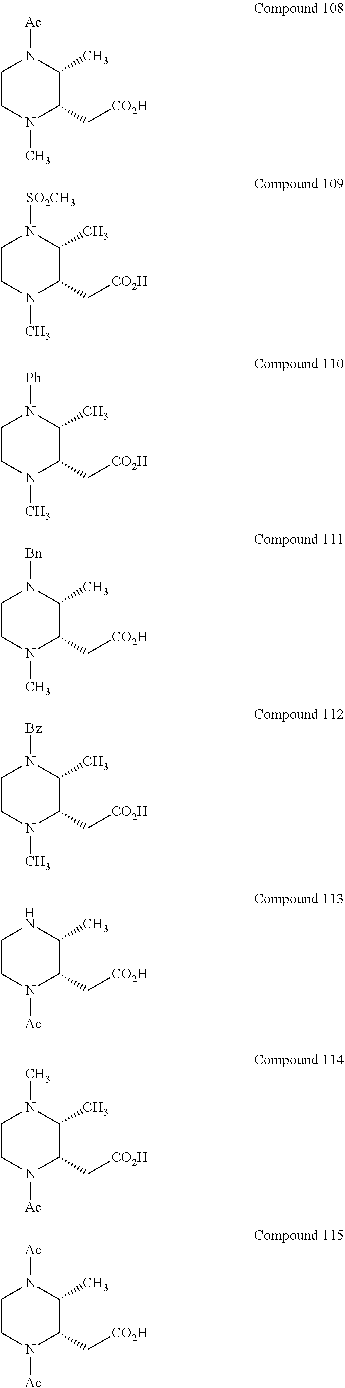

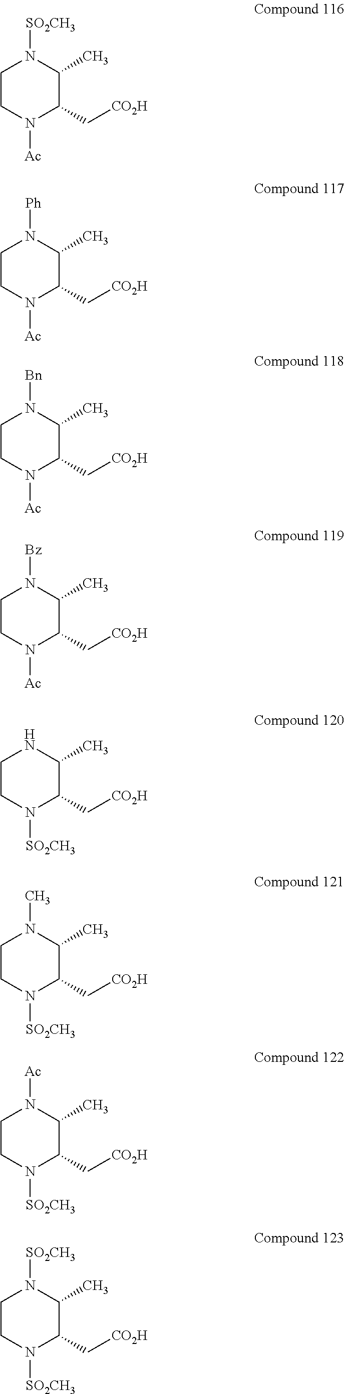

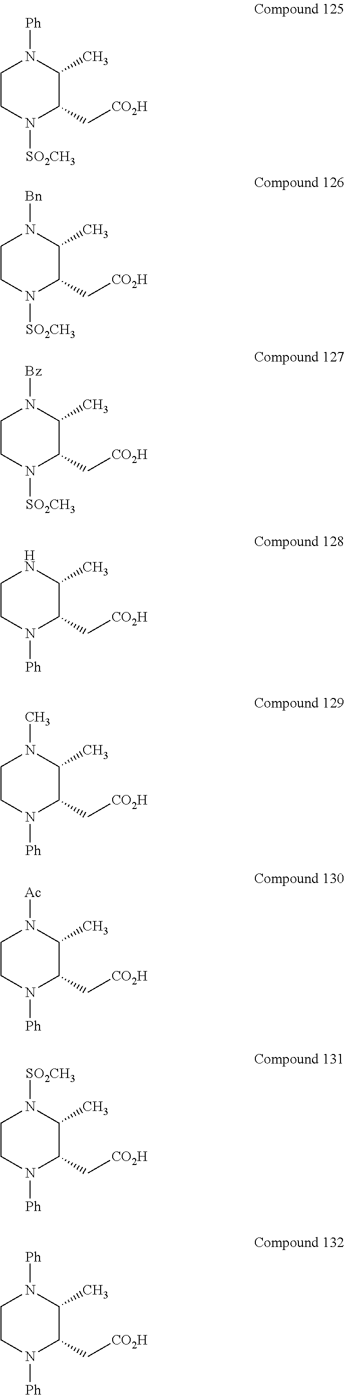

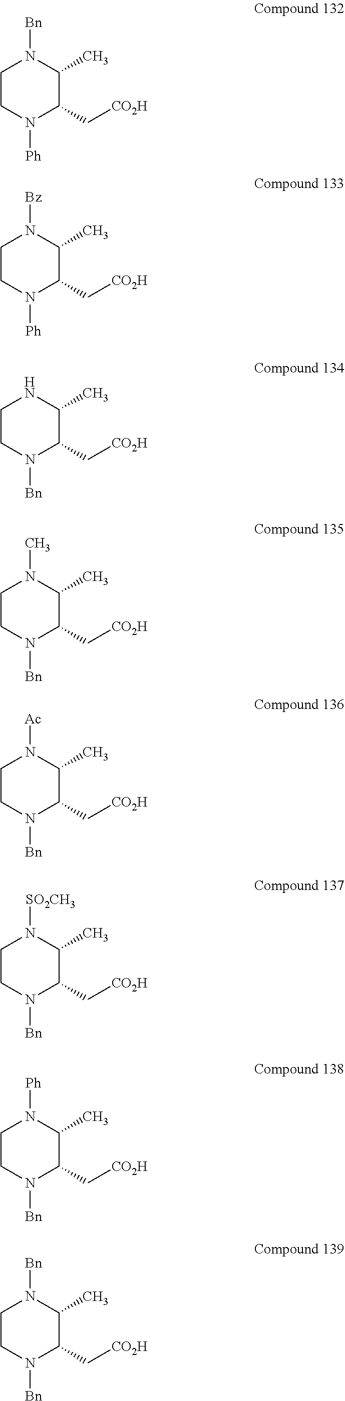

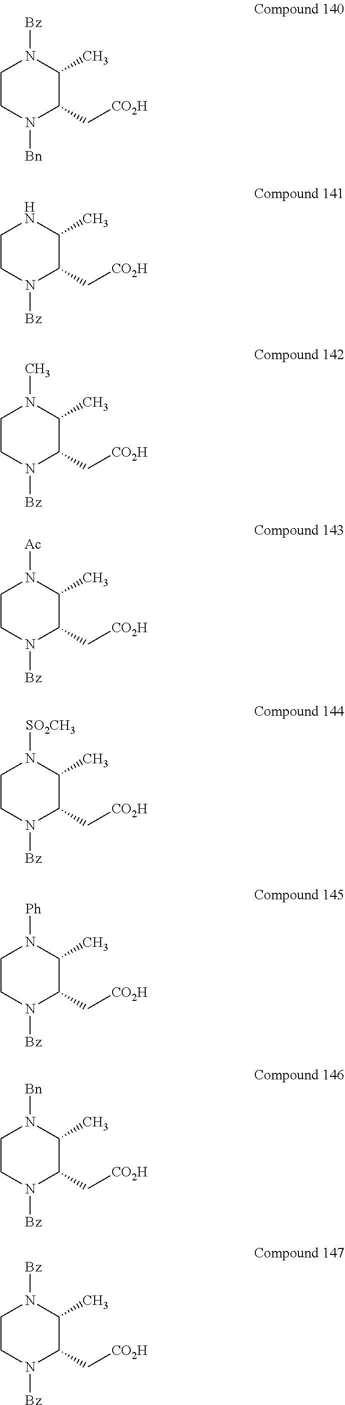

In some cases, the compounds according to Structure I-A are represented by Structure I-A4:

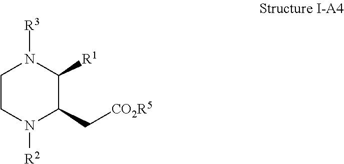

In Structure I-A4, R¹, R², and R³ are as defined above for Formula I. Also in Structure I-A4, R⁵ is hydrogen, substituted or unsubstituted alkyl, substituted or unsubstituted heteroalkyl, substituted or unsubstituted aryl, or substituted or unsubstituted heteroaryl.

Examples of Structure I-A4 include the following compounds:

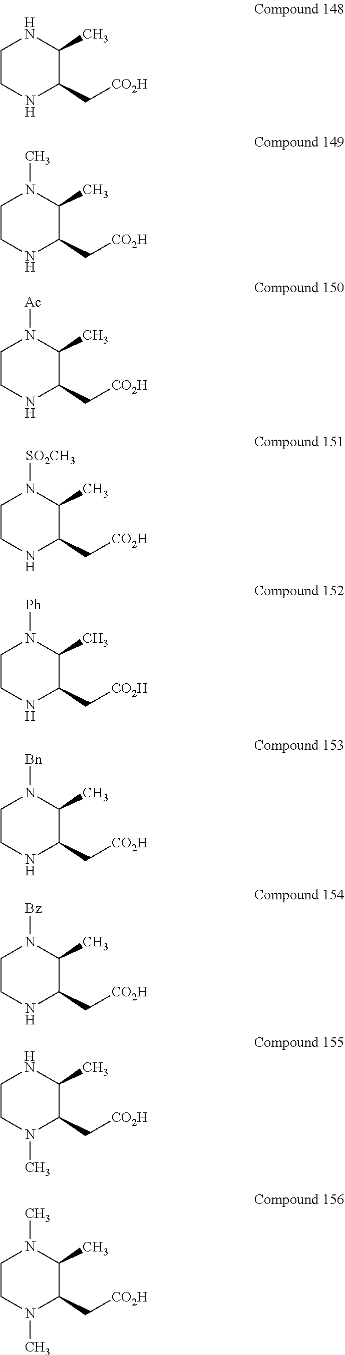

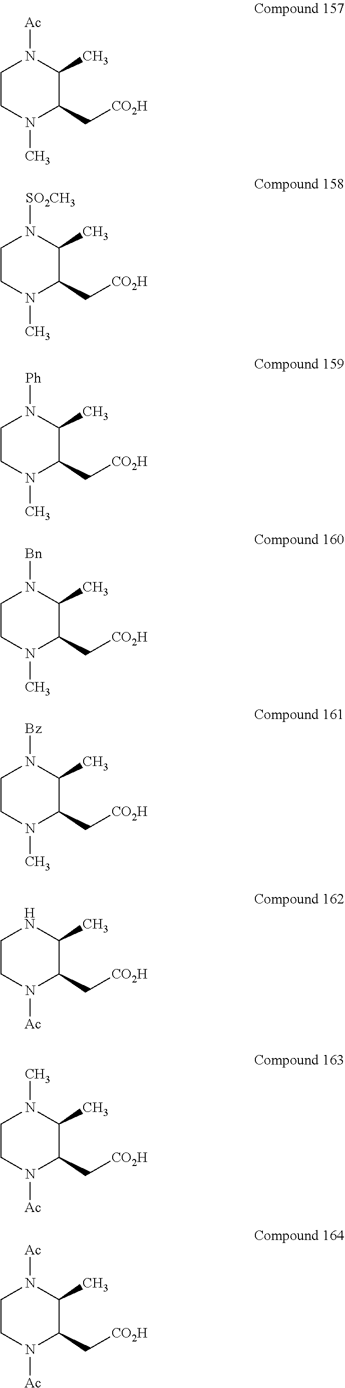

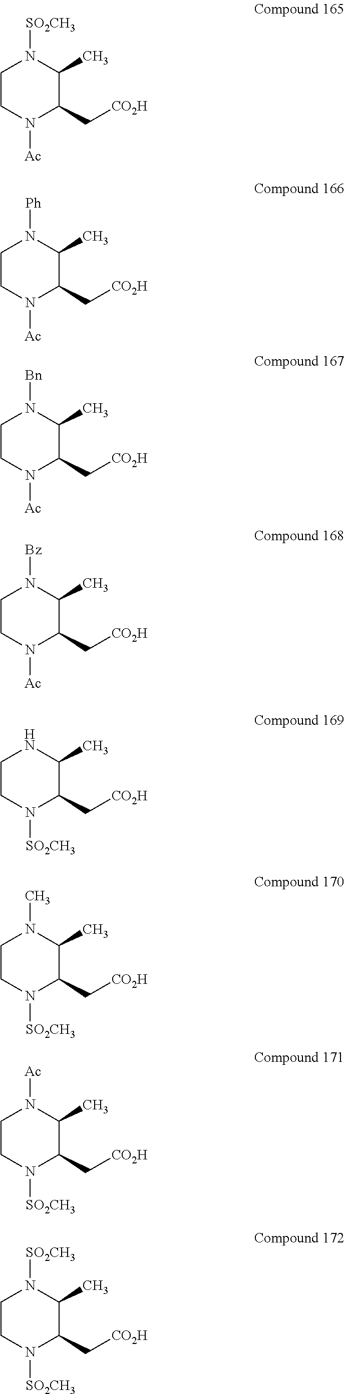

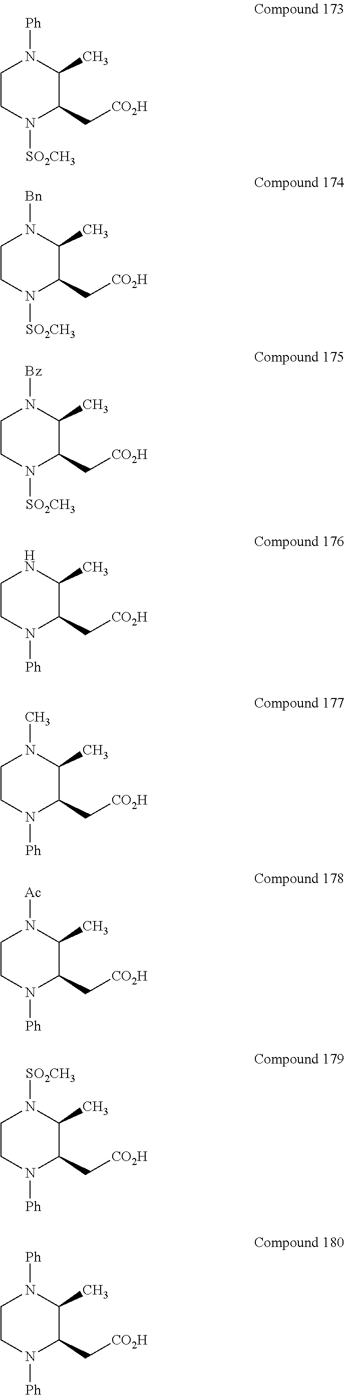

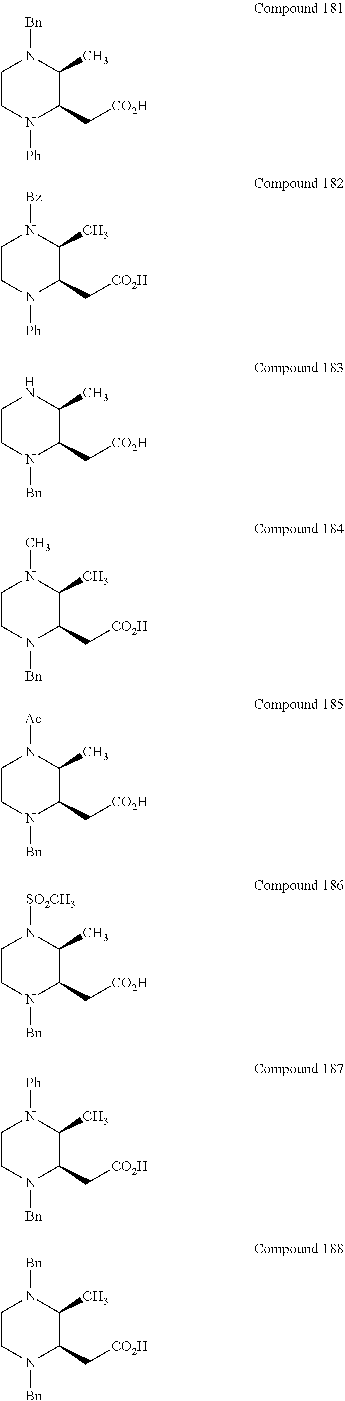

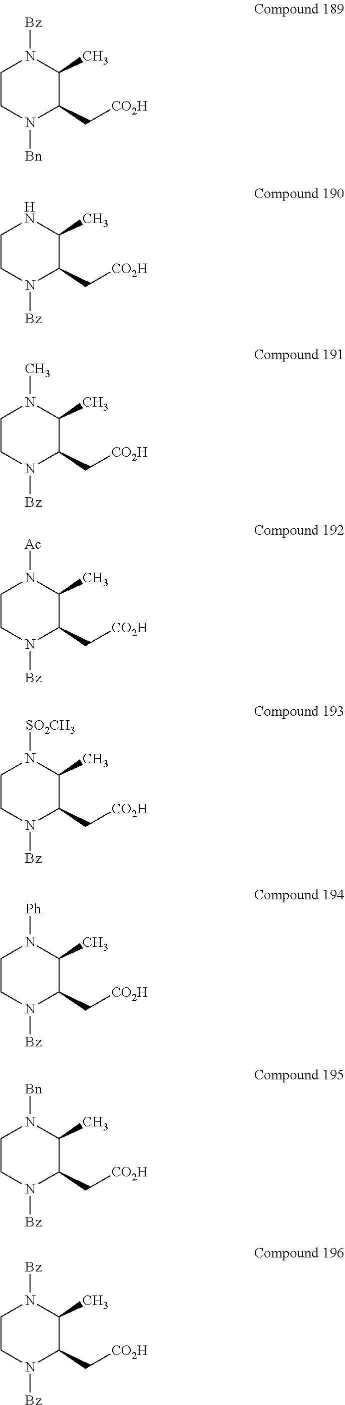

In some cases, the compounds according to Structure I-A are represented by Structure I-A5:

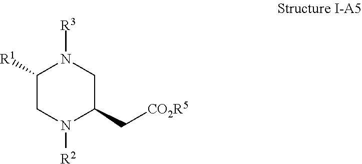

In Structure I-A5, R¹, R², and R³ are as defined above for Formula I. Also in Structure I-A5, R⁵ is hydrogen, substituted or unsubstituted alkyl, substituted or unsubstituted heteroalkyl, substituted or unsubstituted aryl, or substituted or unsubstituted heteroaryl.

Examples of Structure I-A5 include the following compounds:

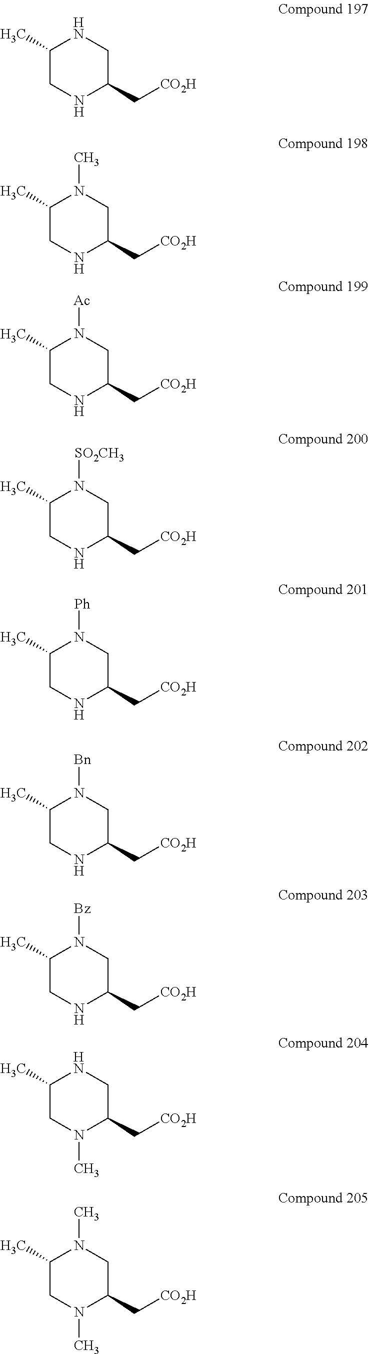

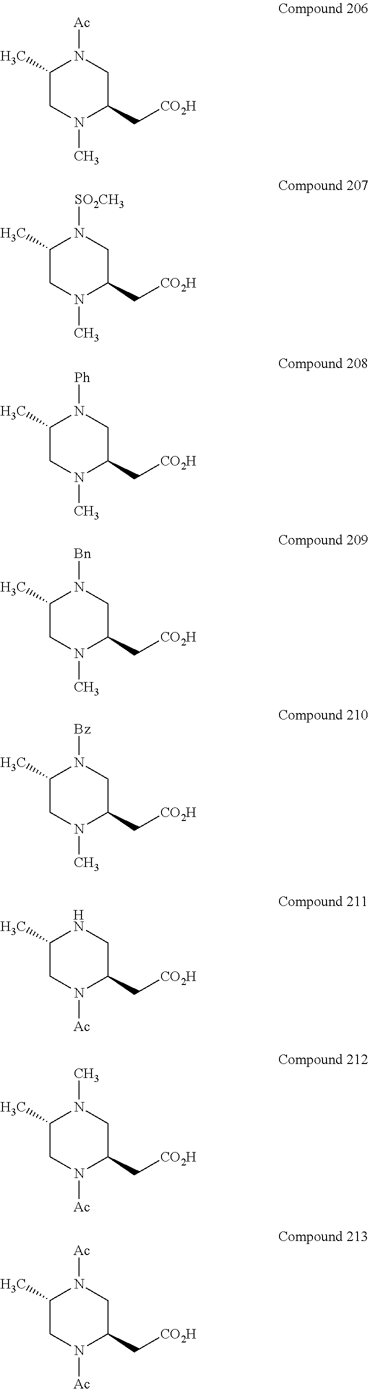

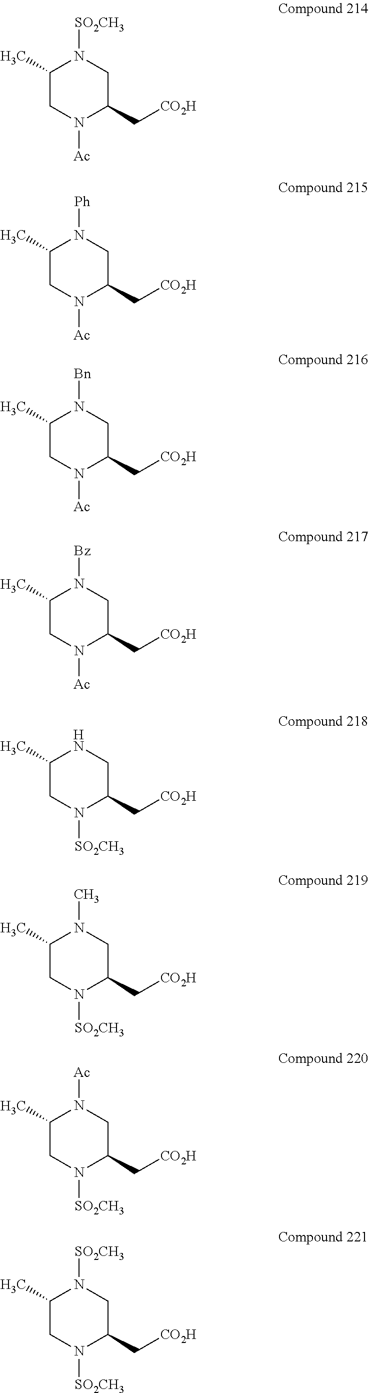

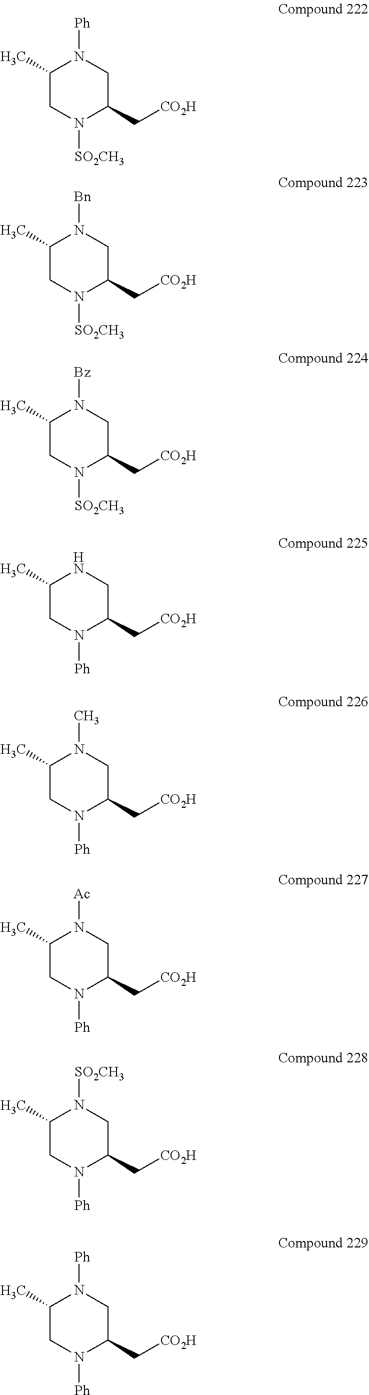

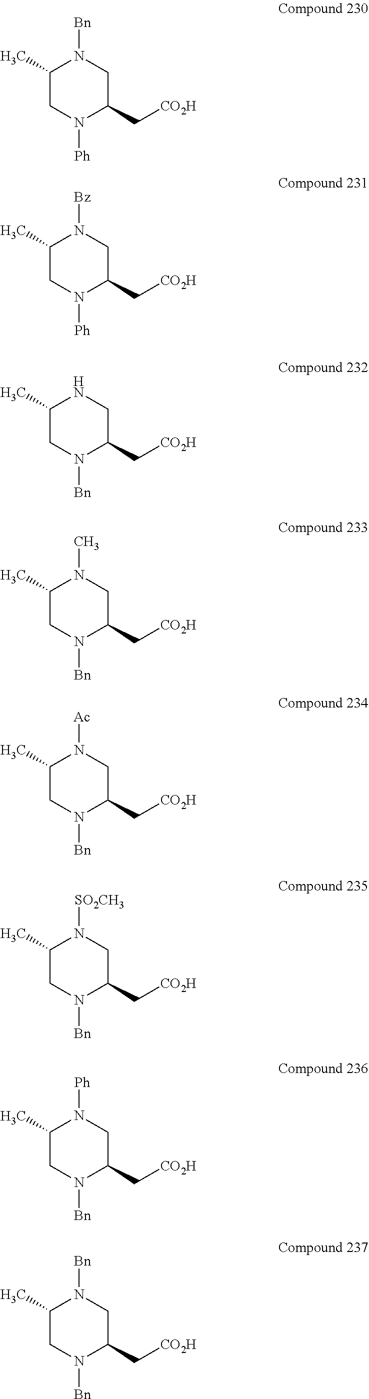

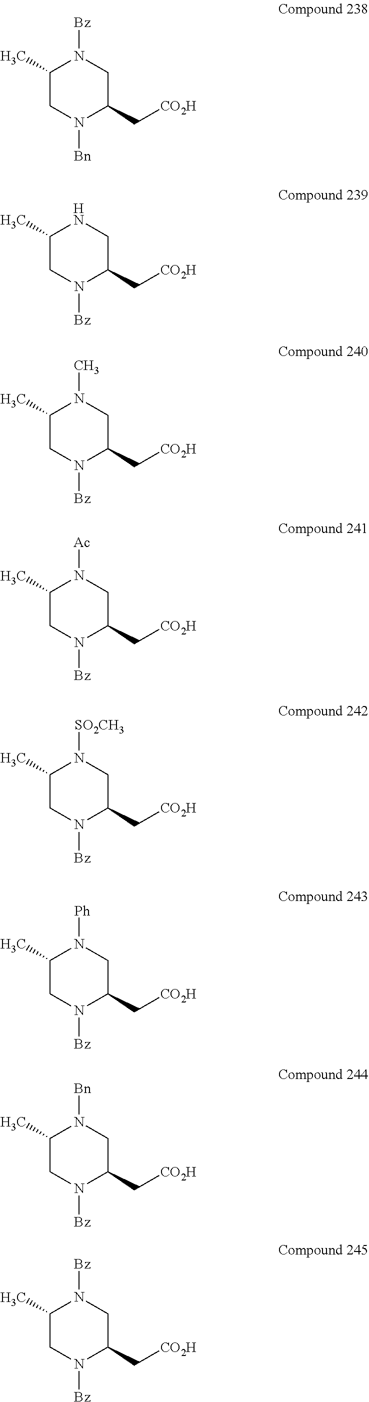

In some cases, the compounds according to Structure I-A are represented by Structure I-A6:

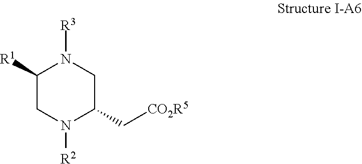

In Structure I-A6, R¹, R², and R³ are as defined above for Formula I. Also in Structure I-A6, R⁵ is hydrogen, substituted or unsubstituted alkyl, substituted or unsubstituted heteroalkyl, substituted or unsubstituted aryl, or substituted or unsubstituted heteroaryl.

Examples of Structure I-A6 include the following compounds:

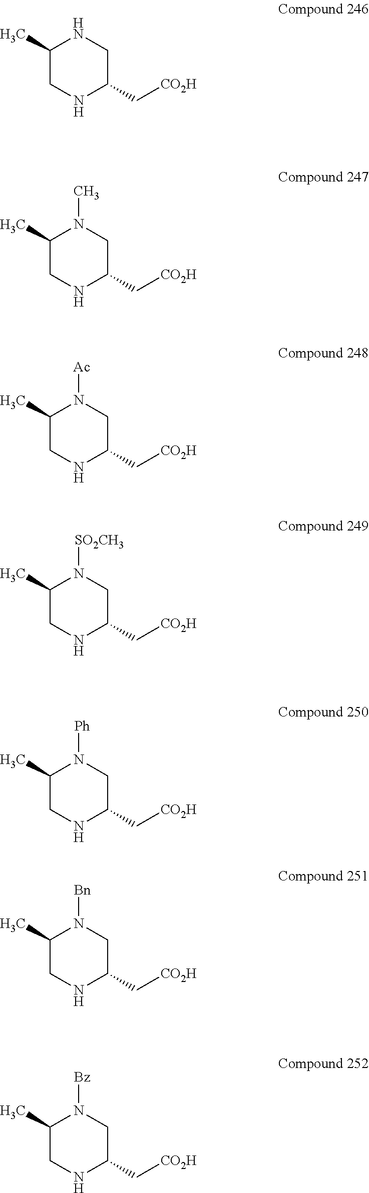

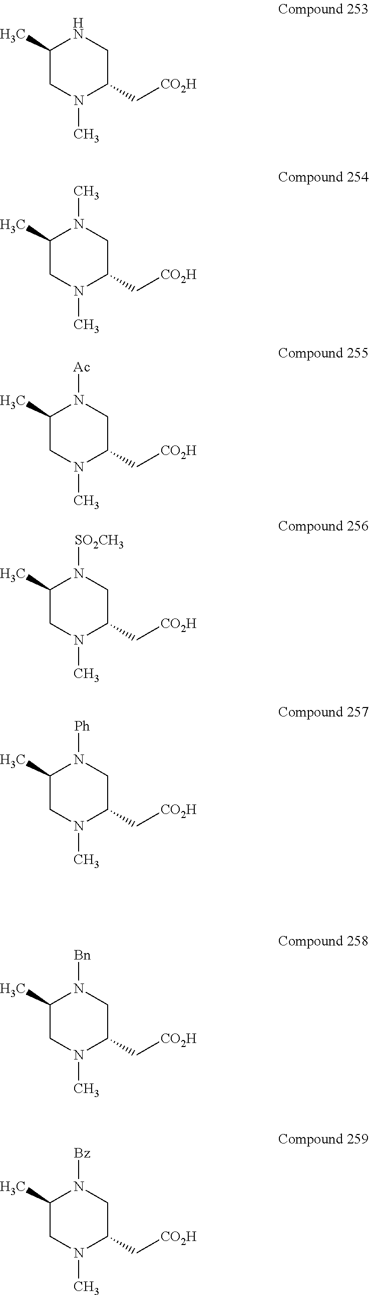

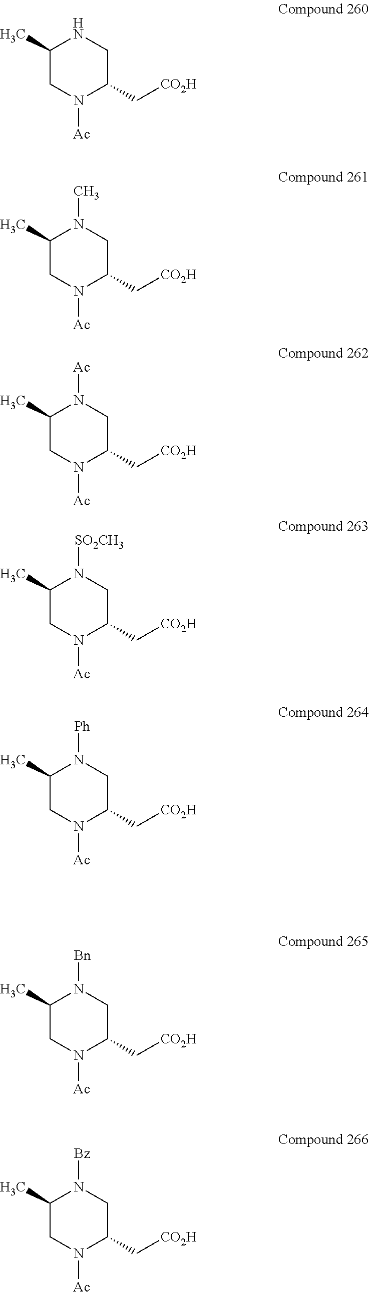

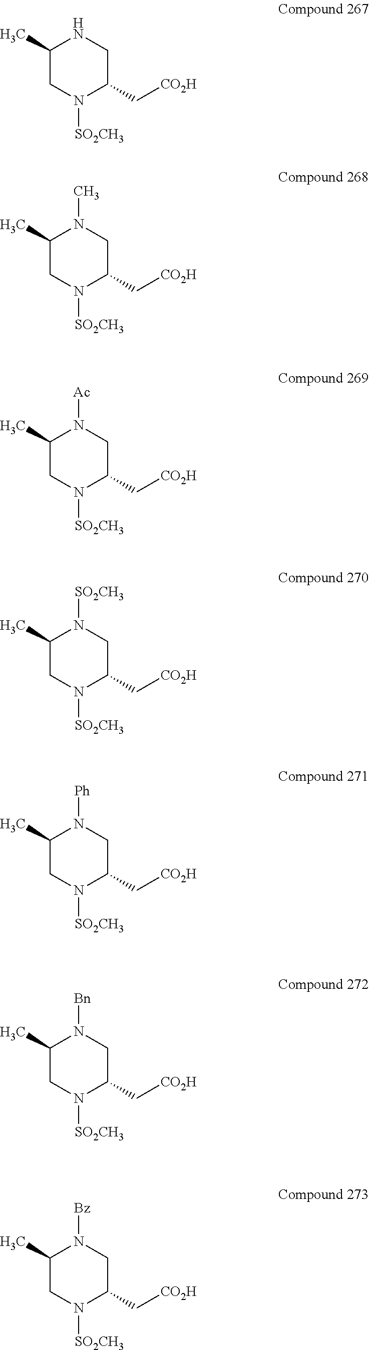

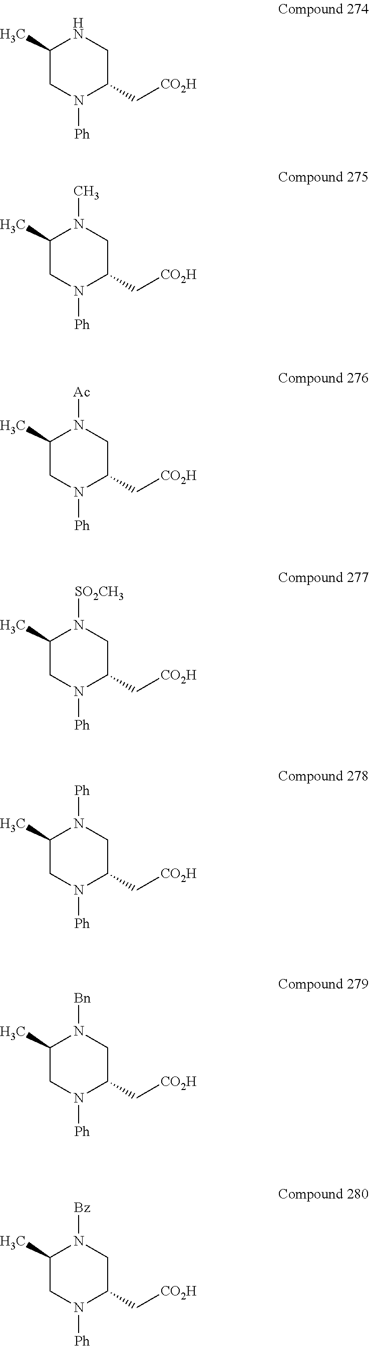

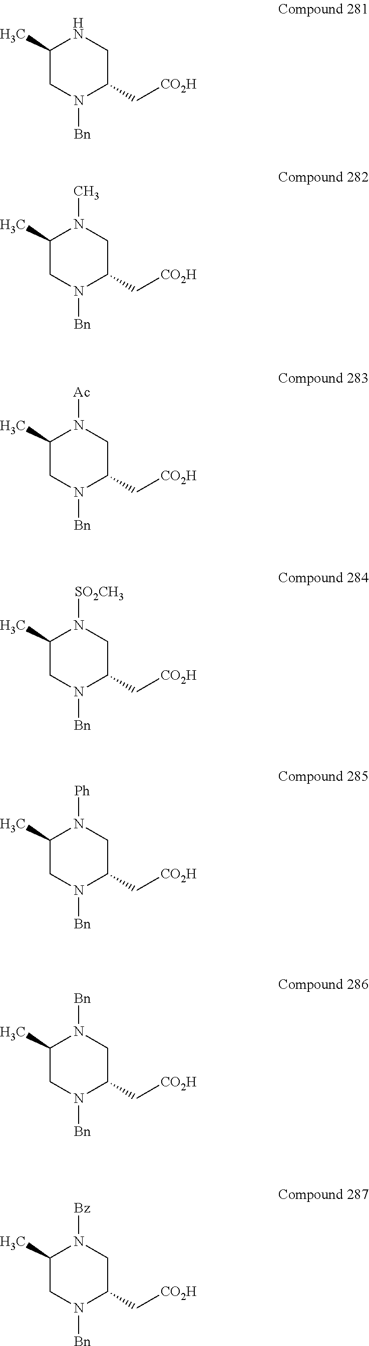

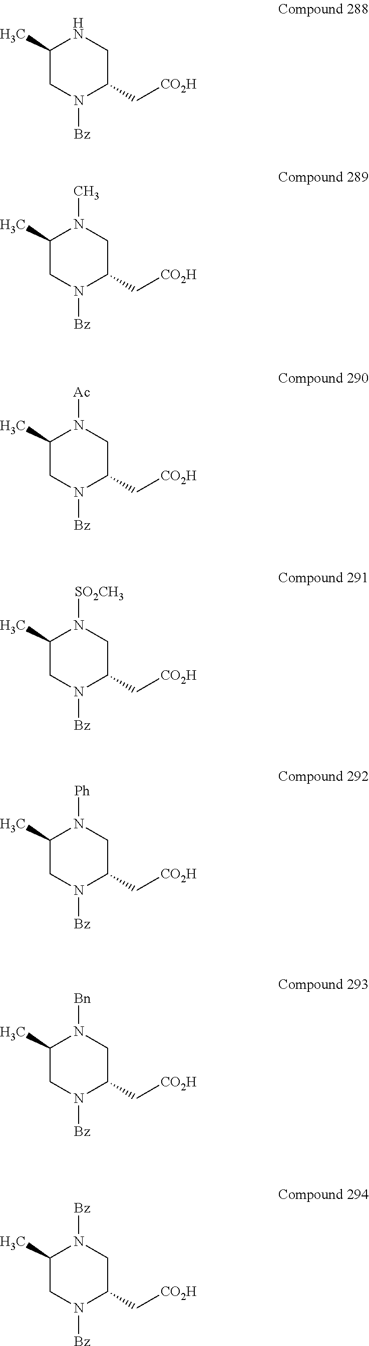

In some cases, the compounds according to Structure I-A are represented by Structure I-A7.

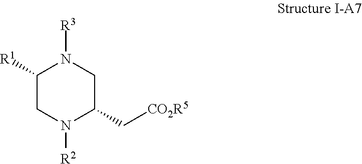

In Structure I-A7, R¹, R², and R³ are as defined above for Formula I. Also in Structure I-A7, R⁵ is hydrogen, substituted or unsubstituted alkyl, substituted or unsubstituted heteroalkyl, substituted or unsubstituted aryl, or substituted or unsubstituted heteroaryl.

Examples of Structure I-A7 include the following compounds:

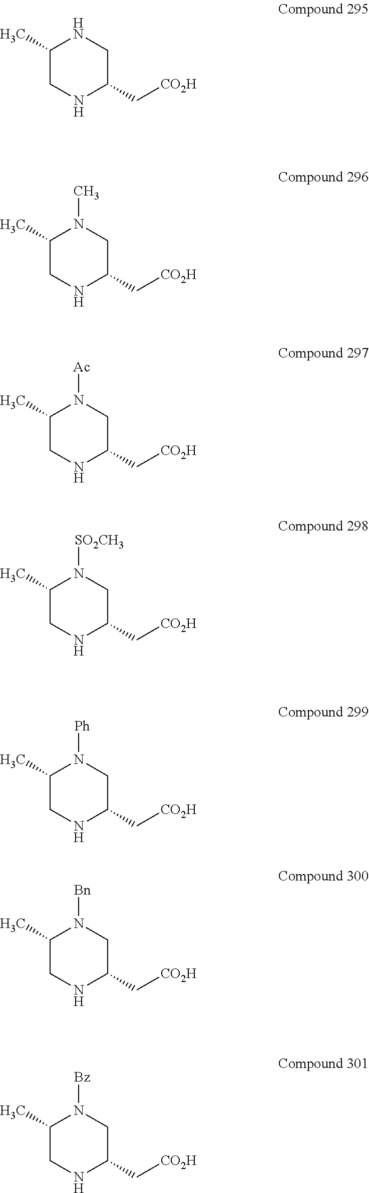

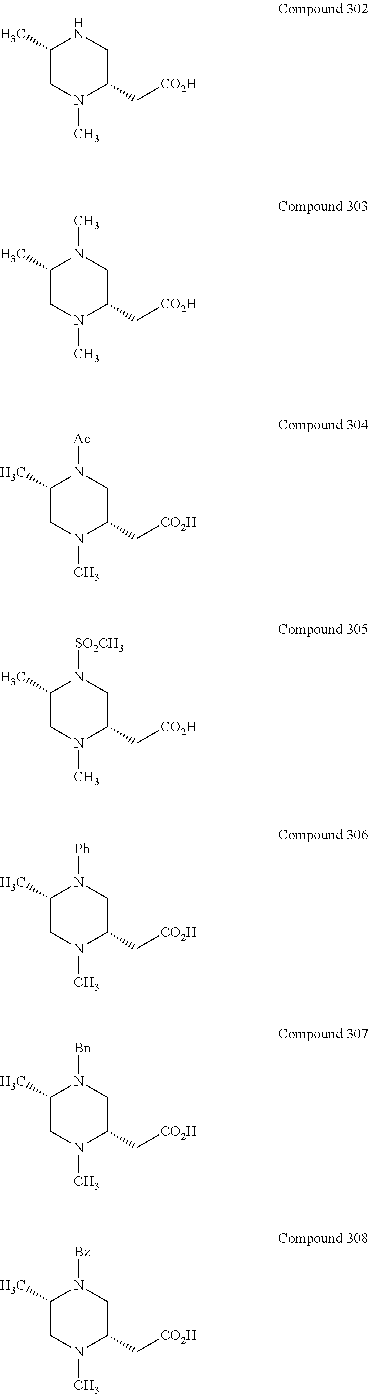

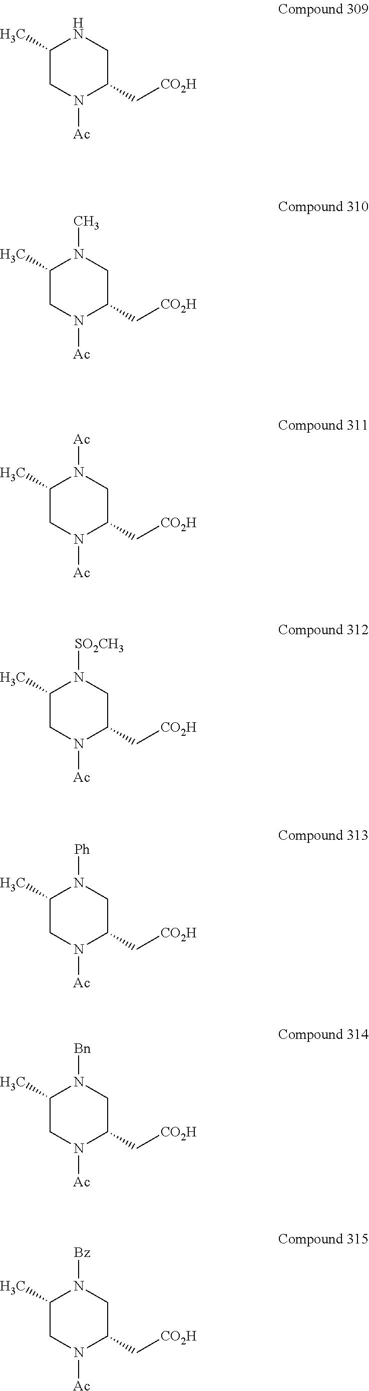

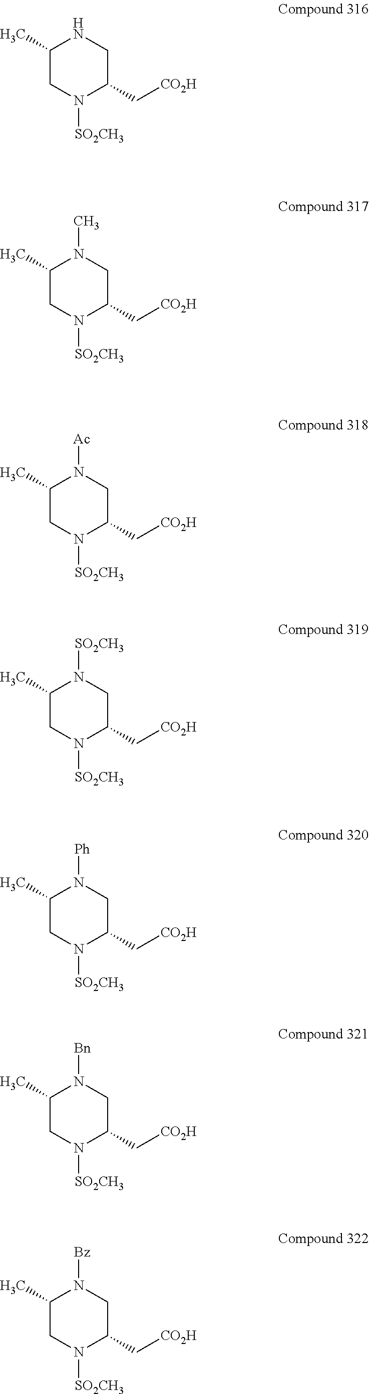

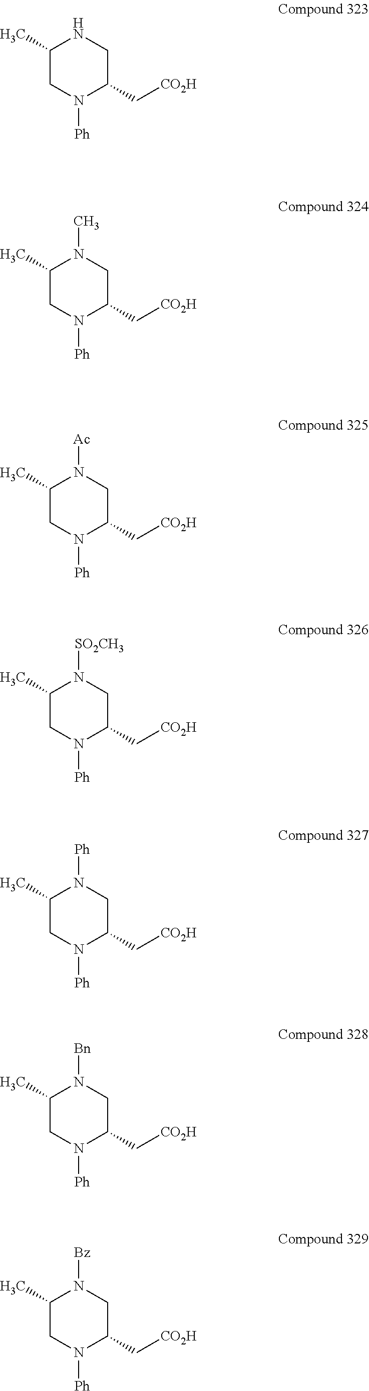

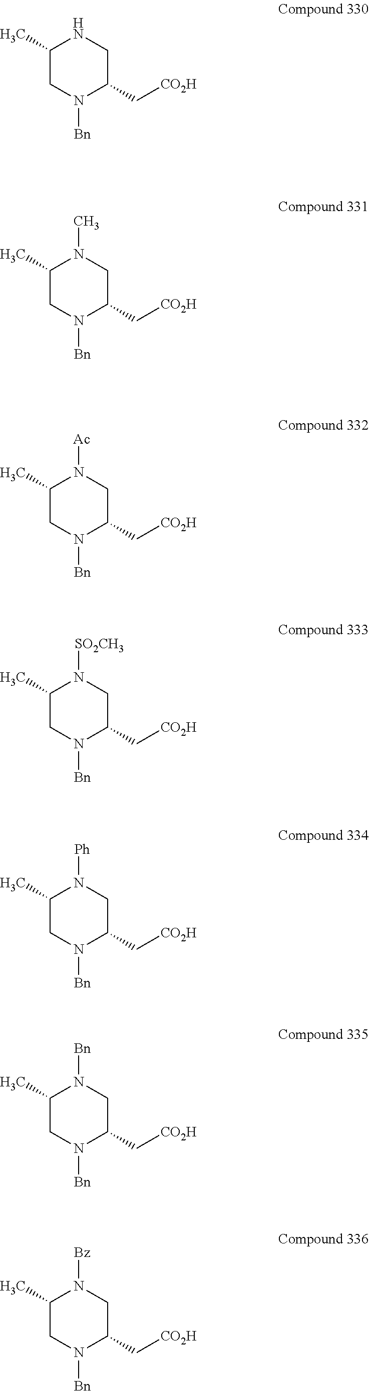

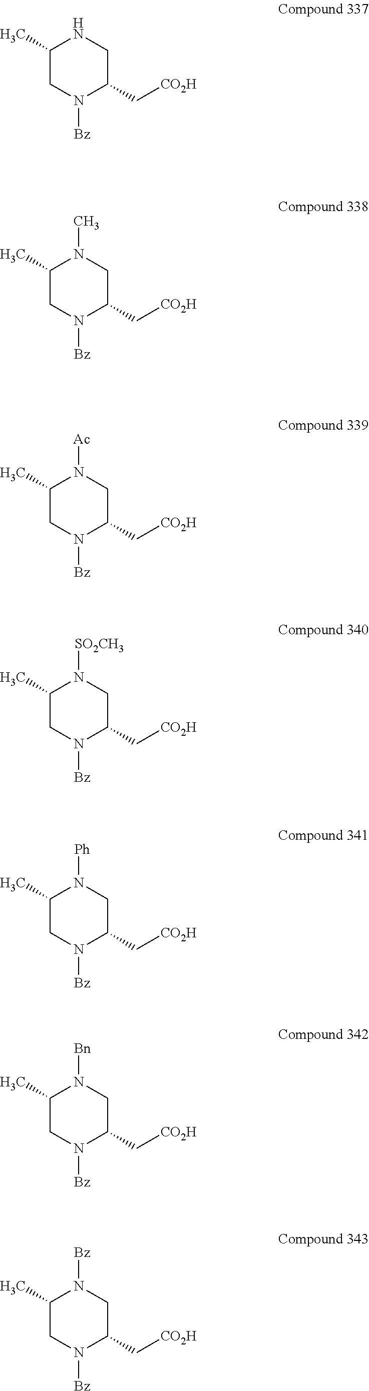

In some cases, the compounds according to Structure I-A are represented by Structure I-A8:

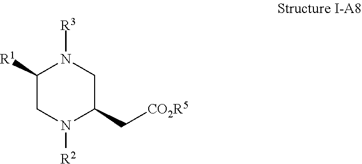

In Structure I-A8, R¹, R², and R³ are as defined above for Formula I. Also in Structure I-A8, R⁵ is hydrogen, substituted or unsubstituted alkyl, substituted or unsubstituted heteroalkyl, substituted or unsubstituted aryl, or substituted or unsubstituted heteroaryl.

Examples of Structure I-A8 include the following compounds:

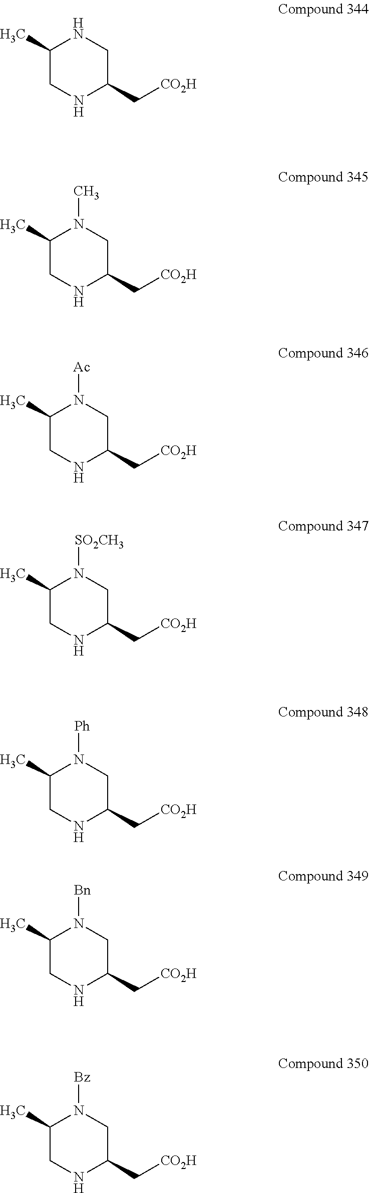

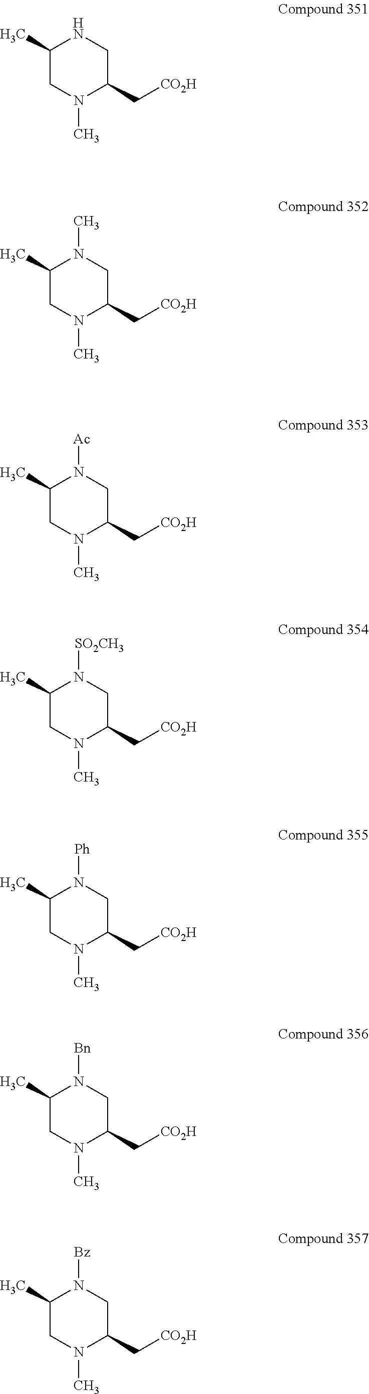

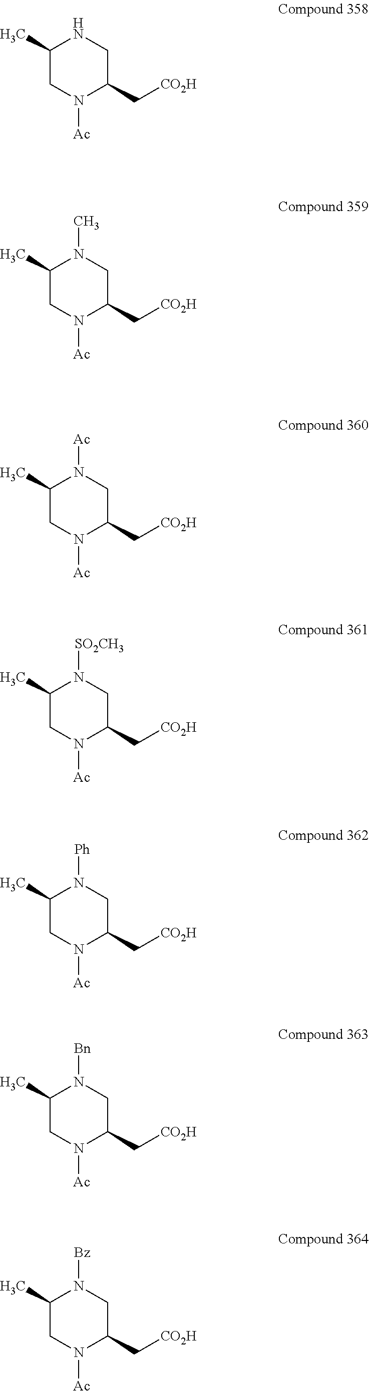

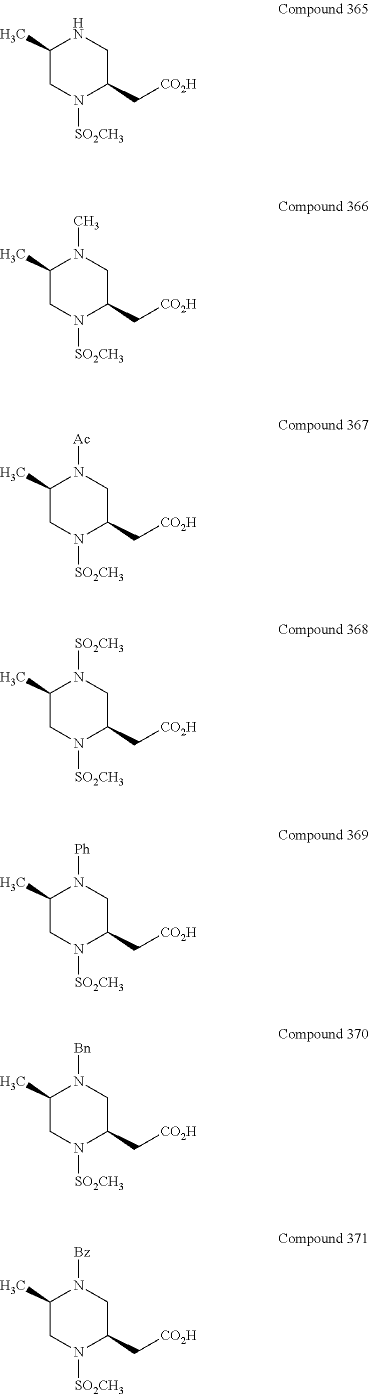

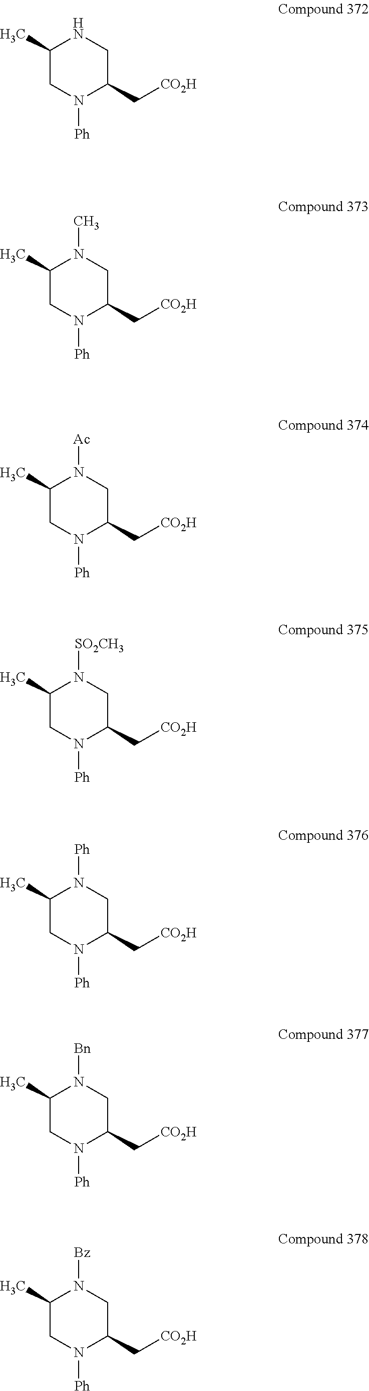

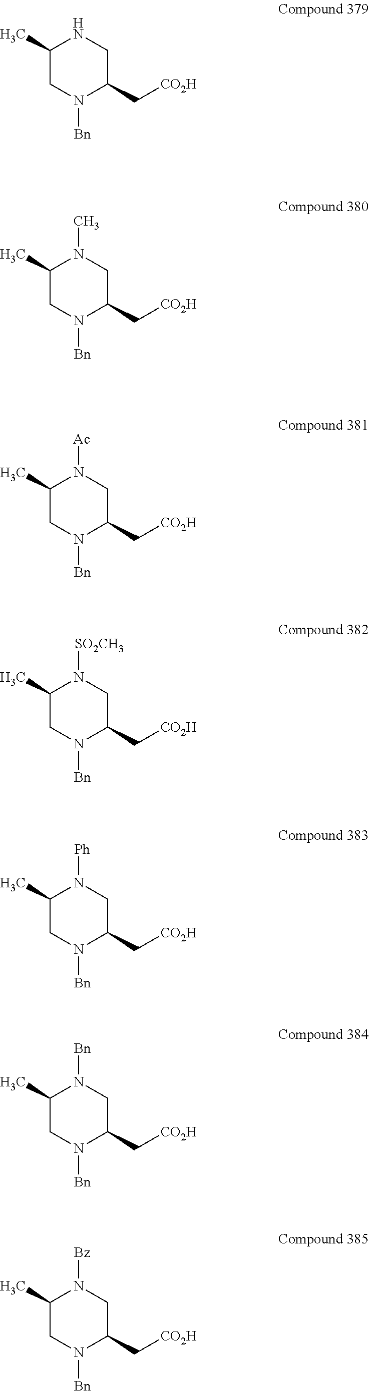

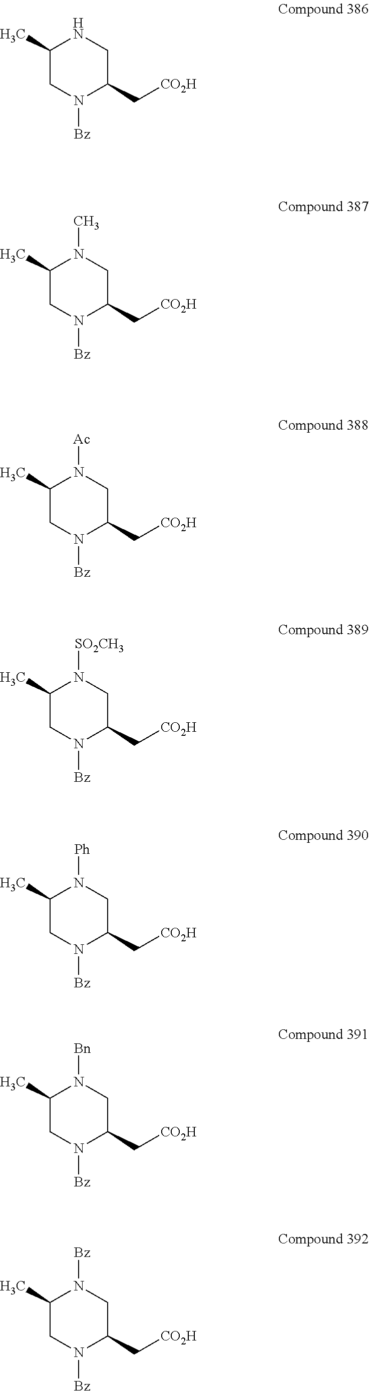

In some cases, the compounds according to Structure I-A are represented by Structure I-A9:

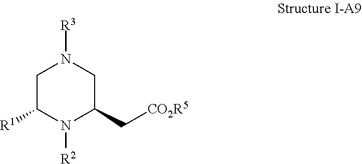

In Structure I-A9, R¹, R², and R³ are as defined above for Formula I. Also in Structure I-A9, R⁵ is hydrogen, substituted or unsubstituted alkyl, substituted or unsubstituted heteroalkyl, substituted or unsubstituted aryl, or substituted or unsubstituted heteroaryl.

Examples of Structure I-A9 include the following compounds:

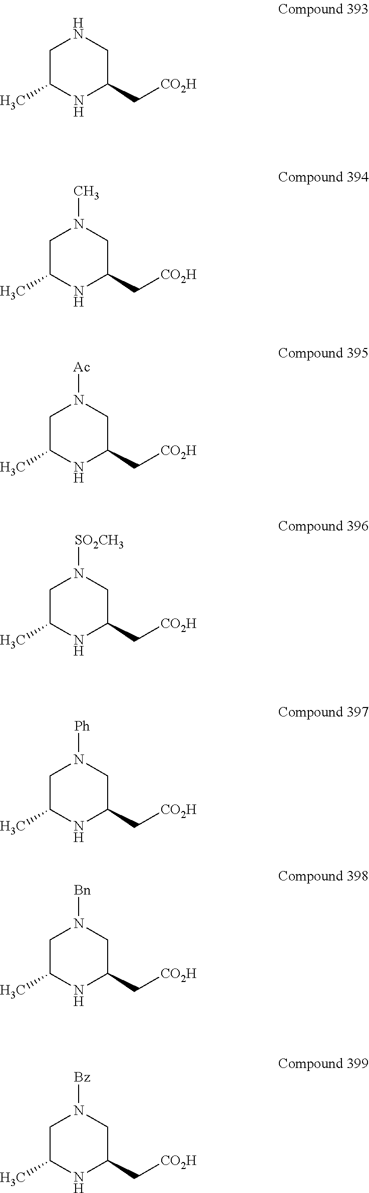

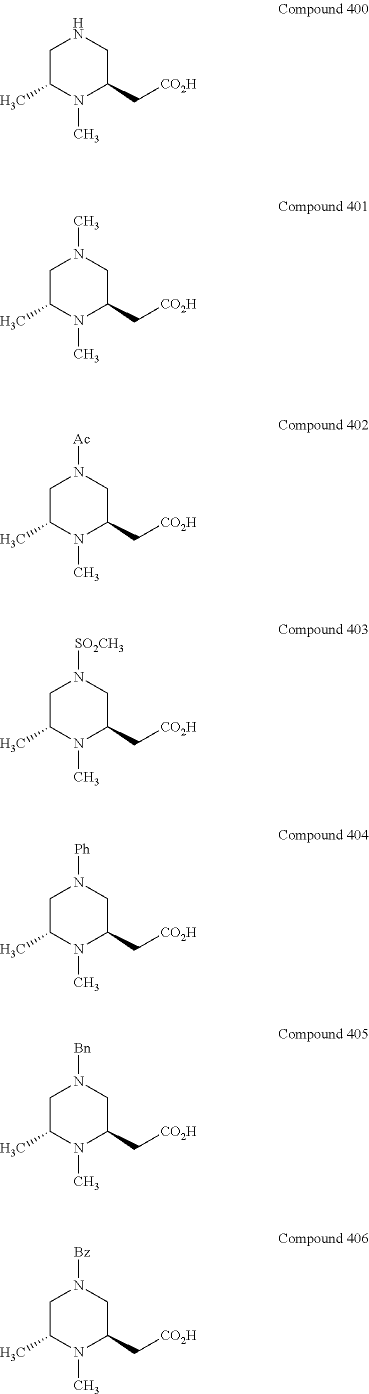

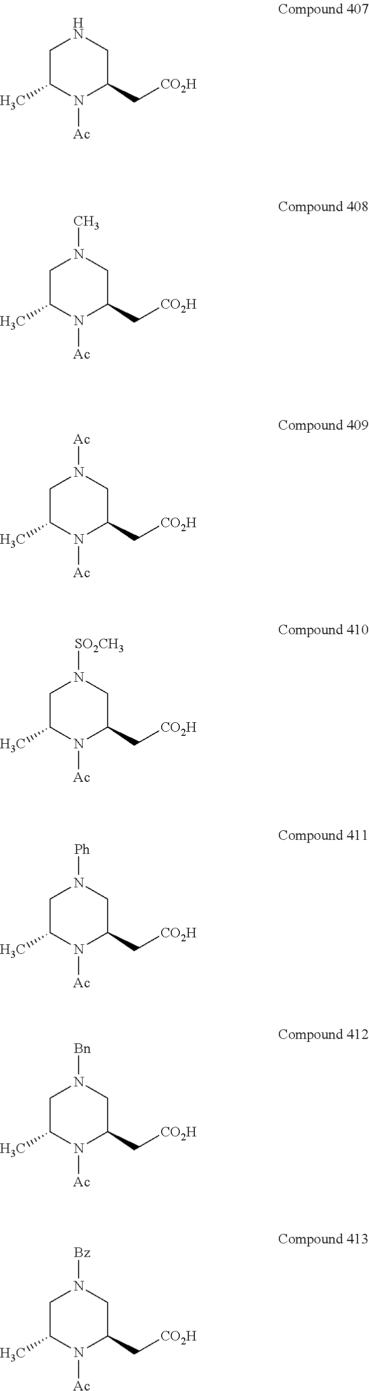

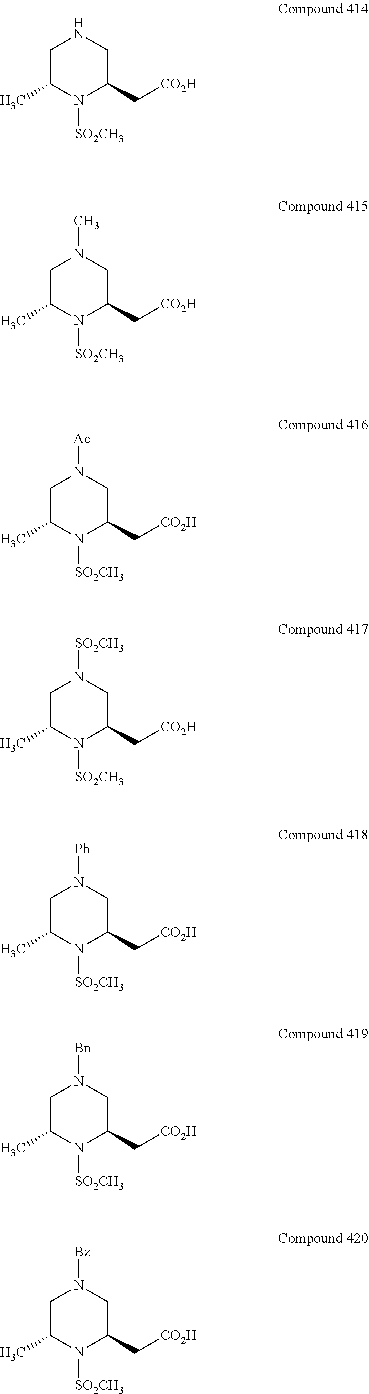

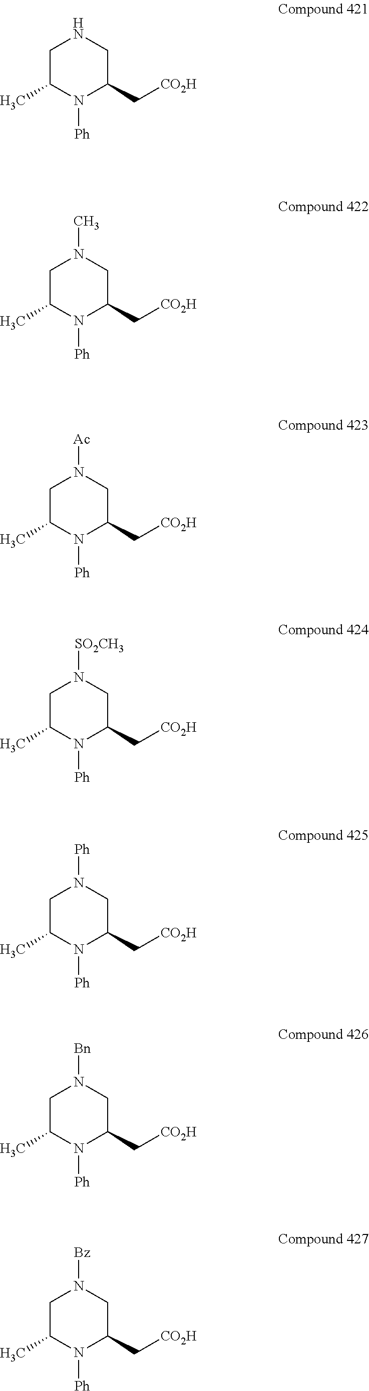

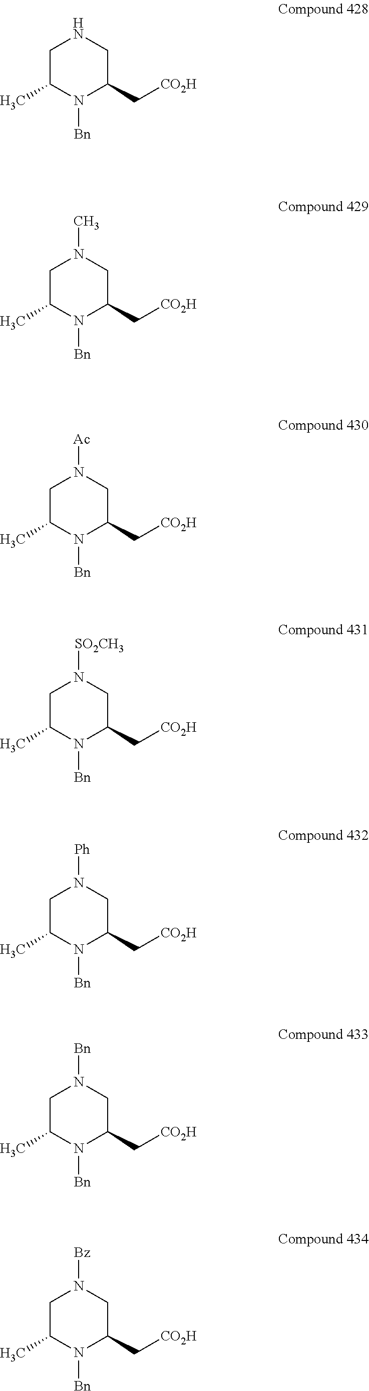

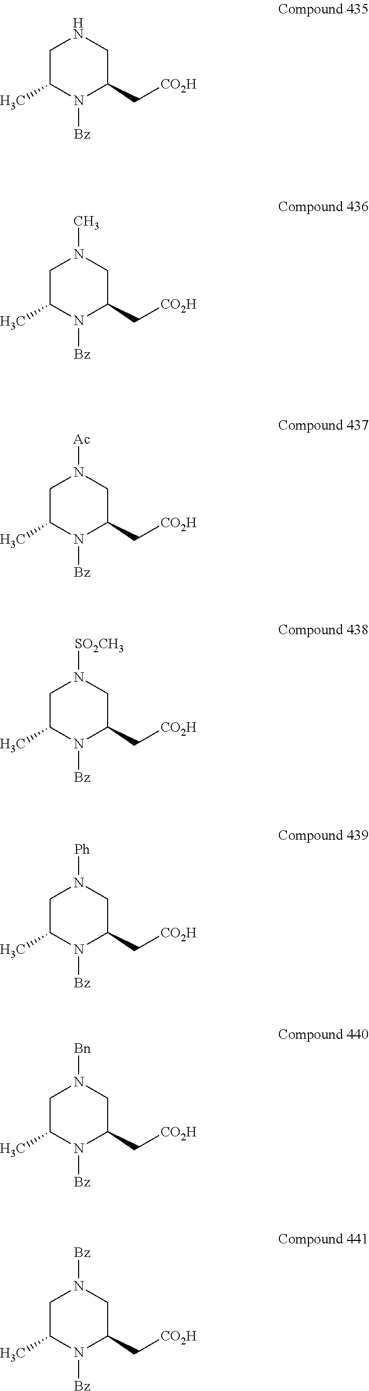

In some cases, the compounds according to Structure I-A are represented by Structure I-A10:

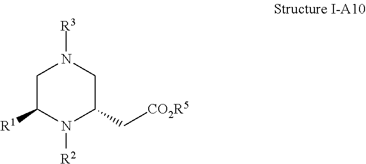

In Structure I-A10, R¹, R², and R³ are as defined above for Formula I. Also in Structure I-A10, R⁵ is hydrogen, substituted or unsubstituted alkyl, substituted or unsubstituted heteroalkyl, substituted or unsubstituted aryl, or substituted or unsubstituted heteroaryl.

Examples of Structure I-A10 include the following compounds:

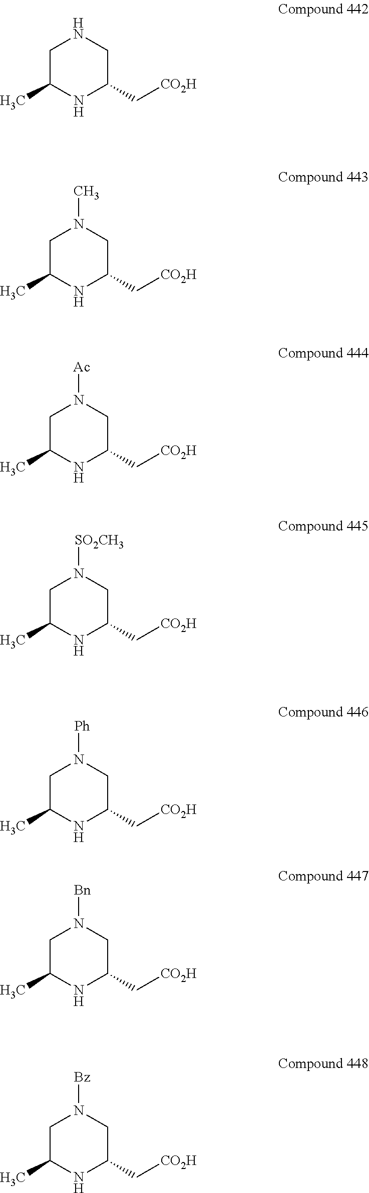

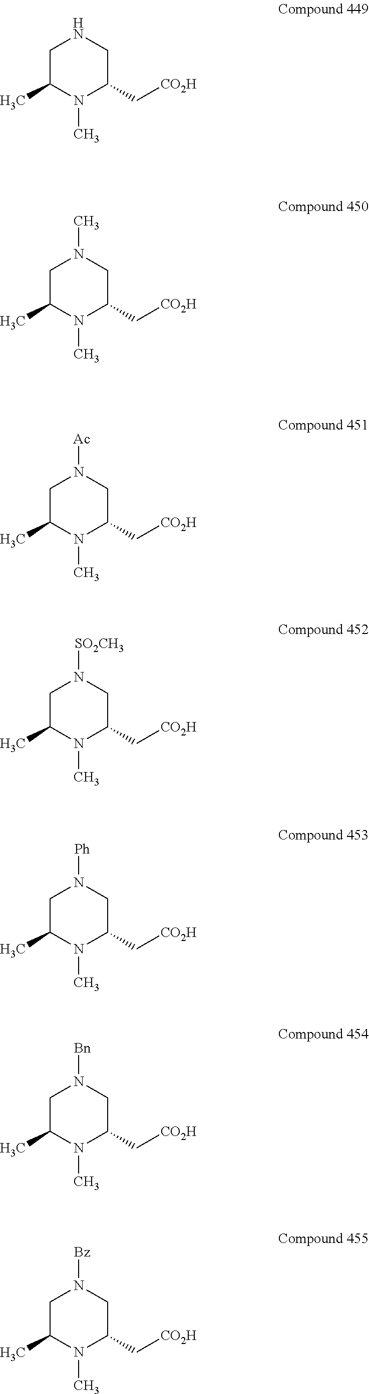

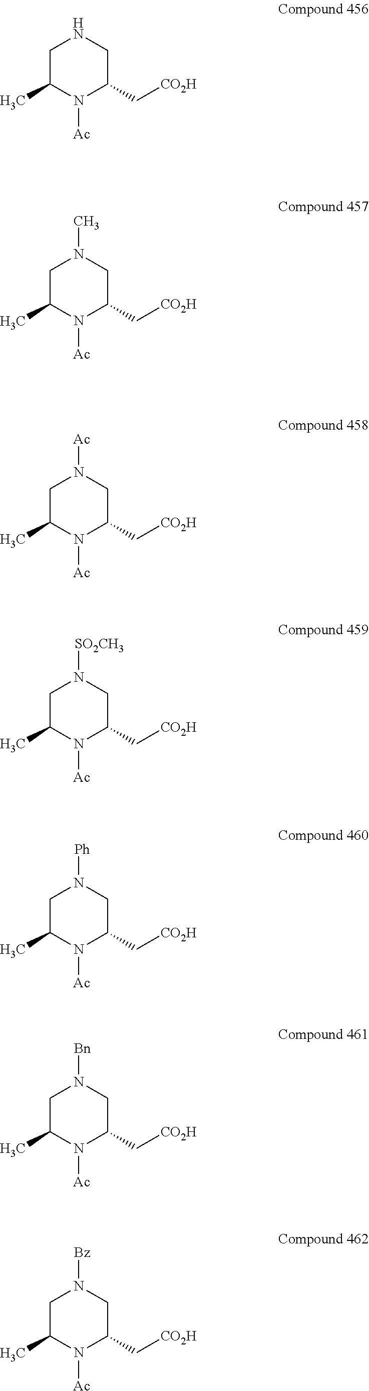

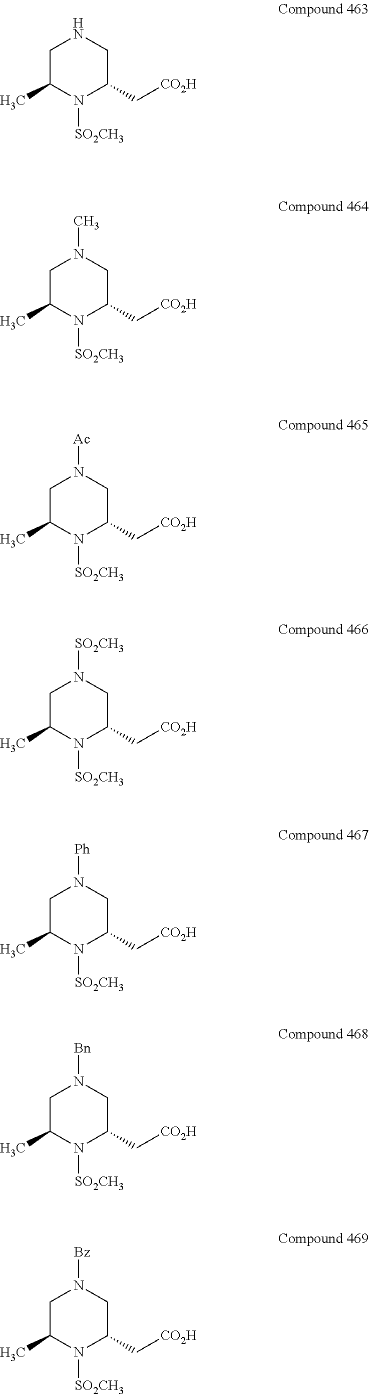

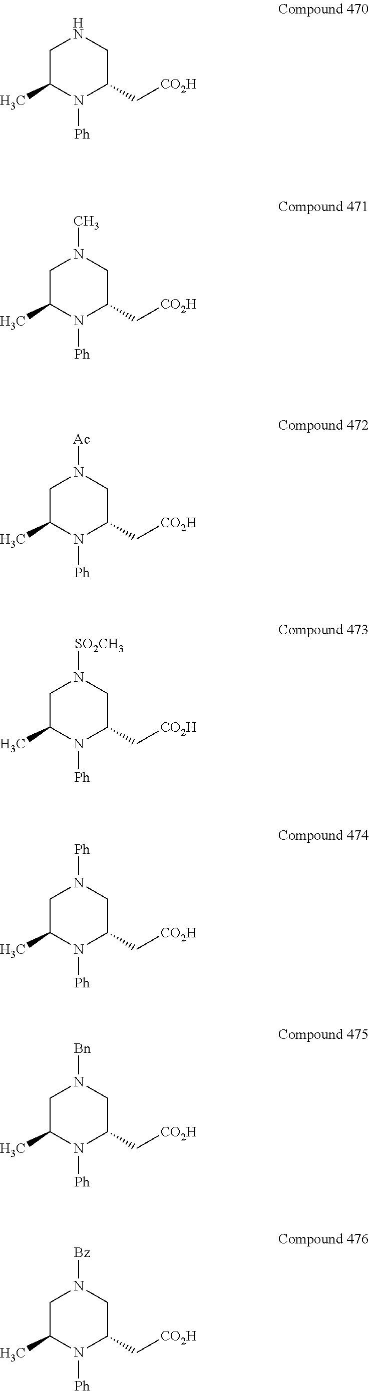

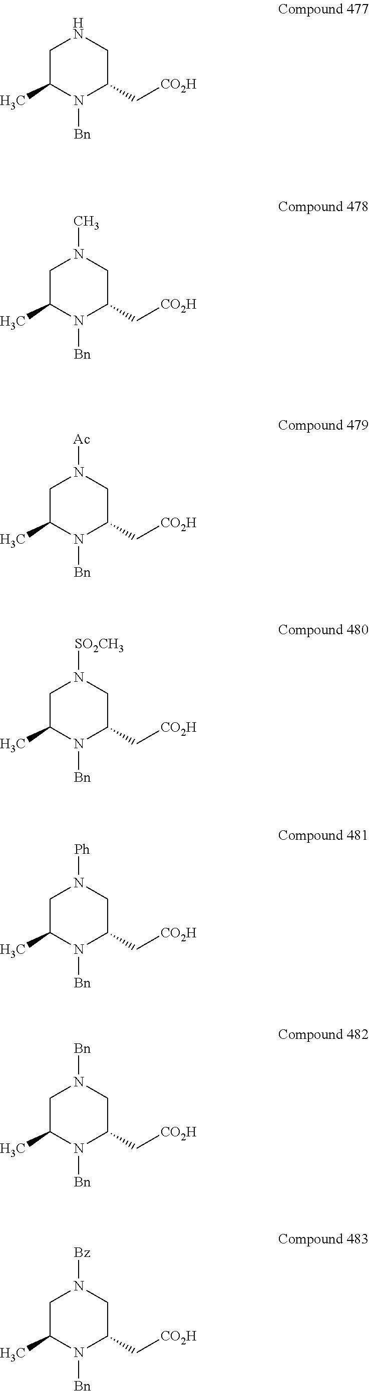

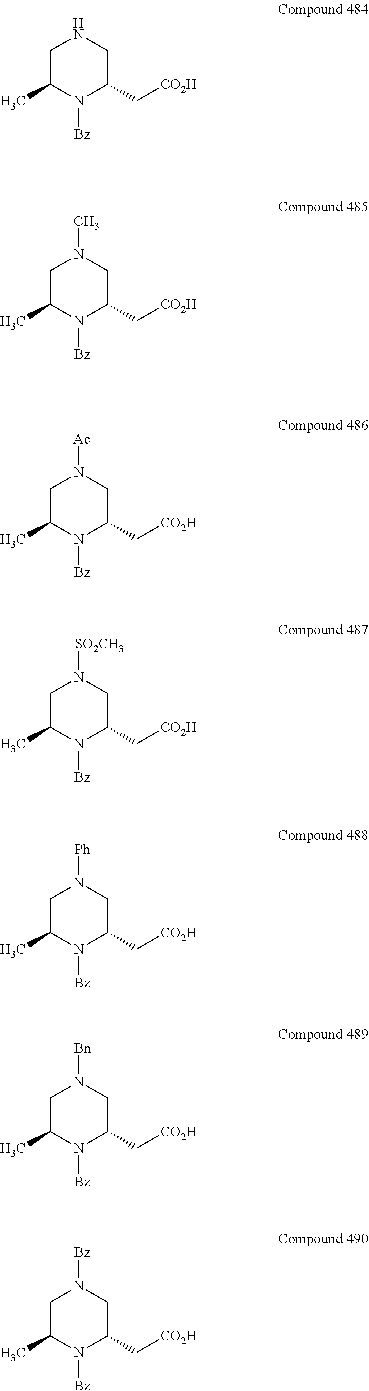

In some cases, the compounds according to Structure I-A are represented by Structure I-A11:

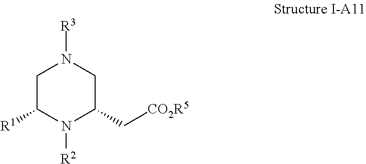

In Structure I-A11, R¹, R², and R³ are as defined above for Formula I. Also in Structure I-A11, R⁵ is hydrogen, substituted or unsubstituted alkyl, substituted or unsubstituted heteroalkyl, substituted or unsubstituted aryl, or substituted or unsubstituted heteroaryl.

Examples of Structure I-A11 include the following compounds:

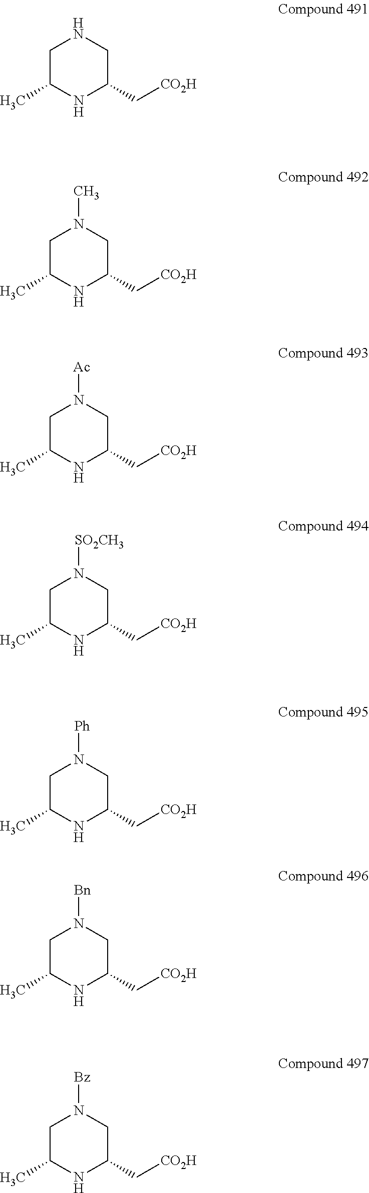

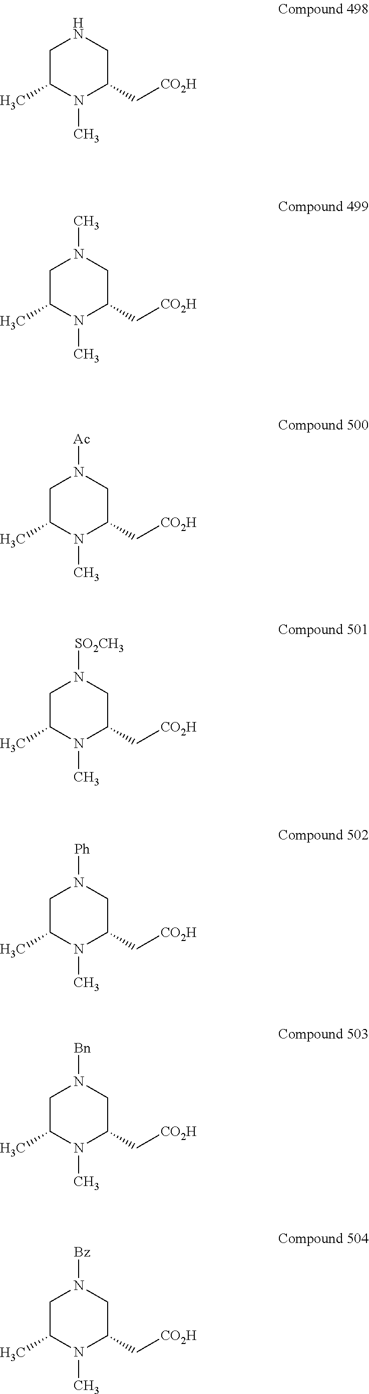

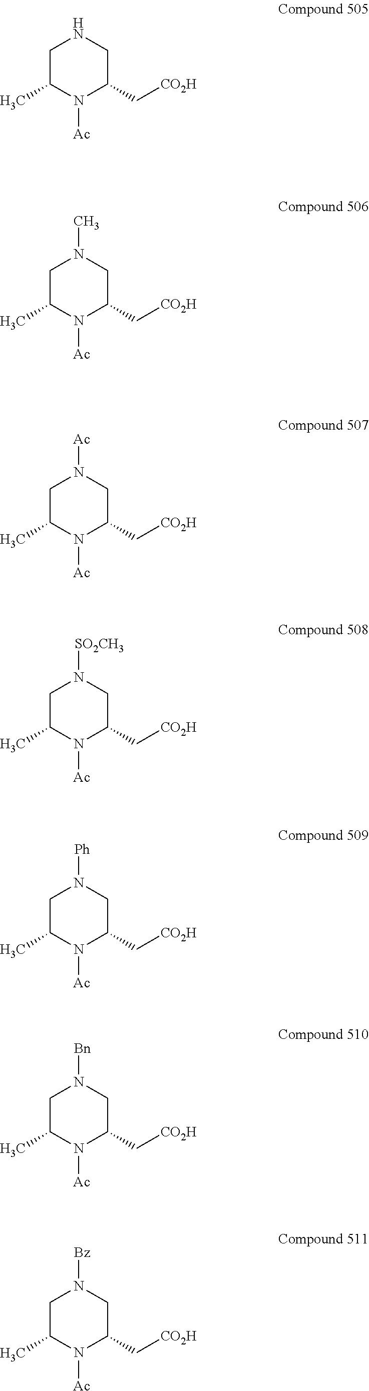

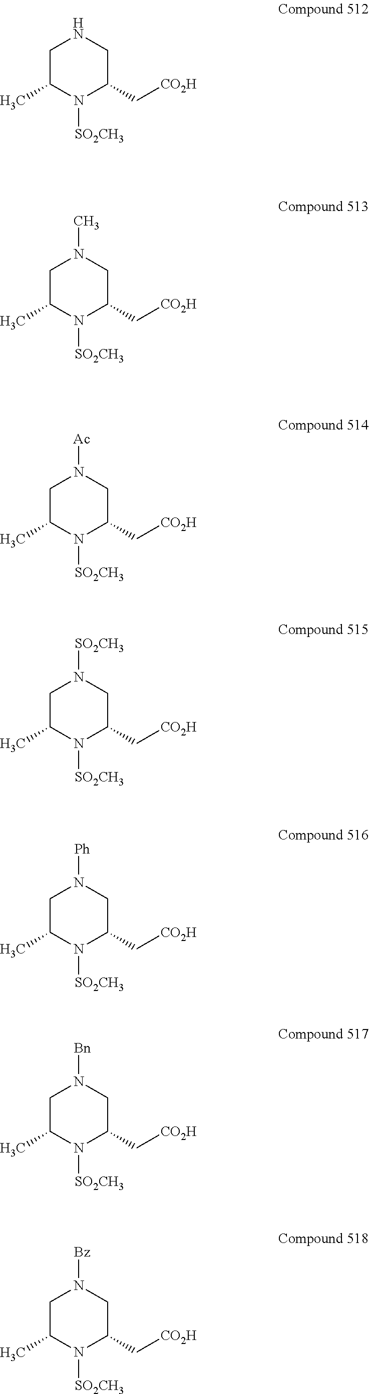

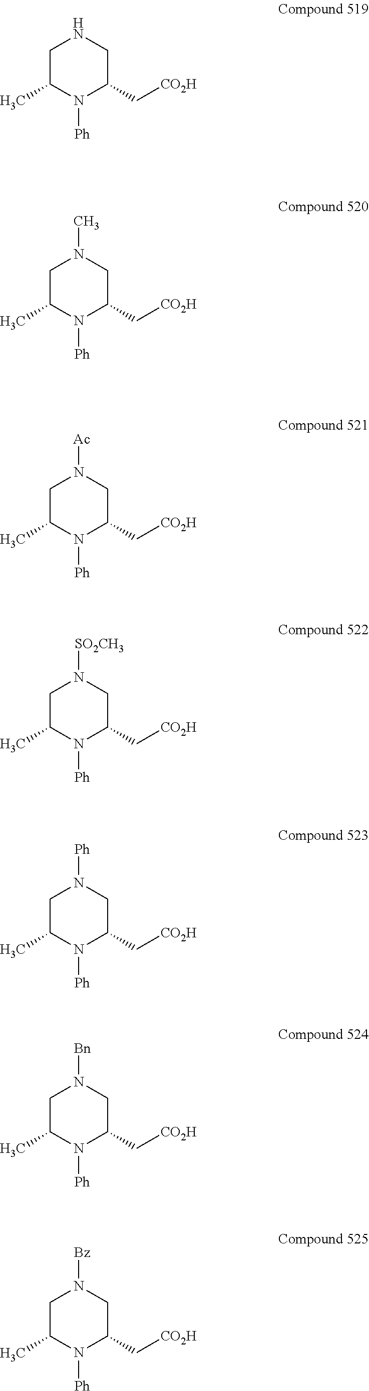

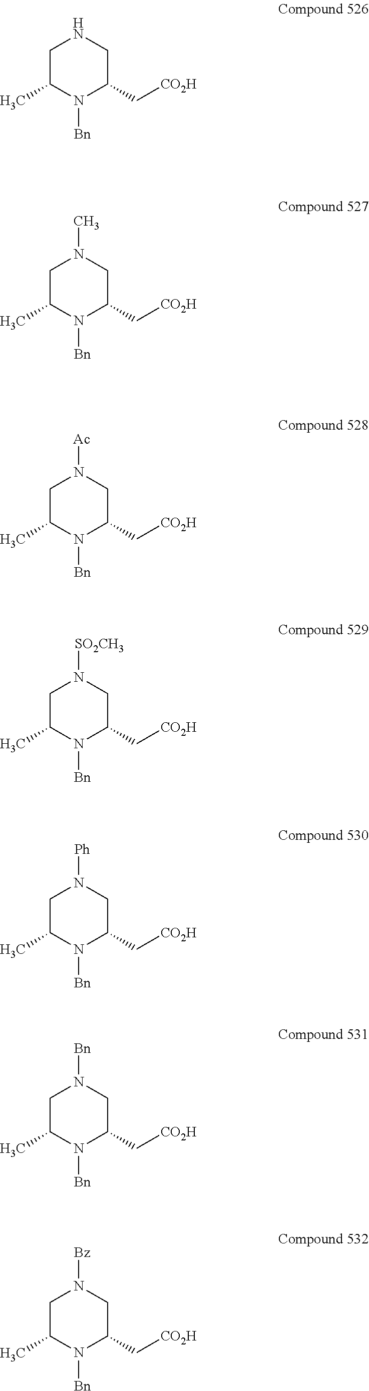

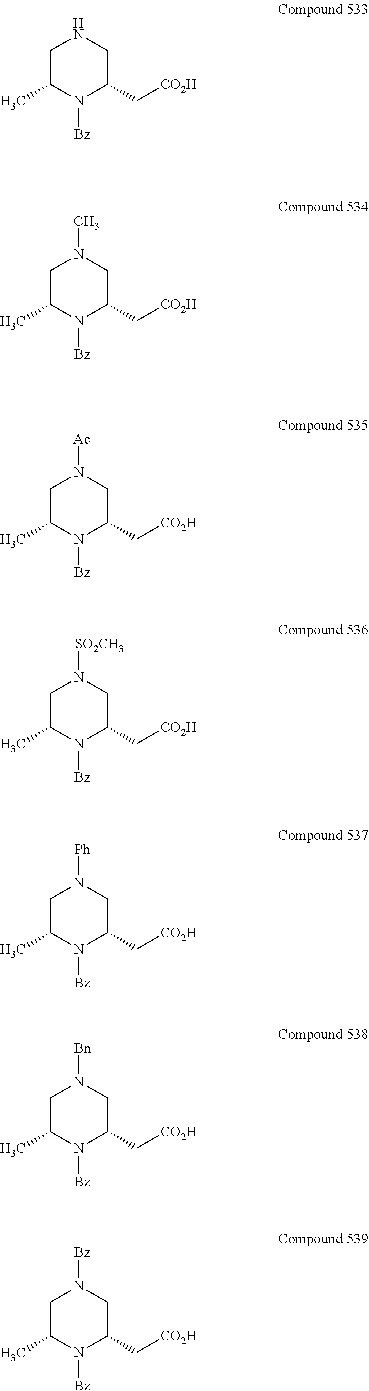

In some cases, the compounds according to Structure I-A are represented by Structure I-A12:

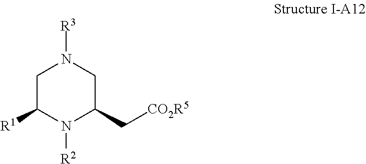

In Structure I-A12, R¹, R², and R³ are as defined above for Formula I. Also in Structure I-A12, R⁵ is hydrogen, substituted or unsubstituted alkyl, substituted or unsubstituted heteroalkyl, substituted or unsubstituted aryl, or substituted or unsubstituted heteroaryl.

Examples of Structure I-A12 include the following compounds:

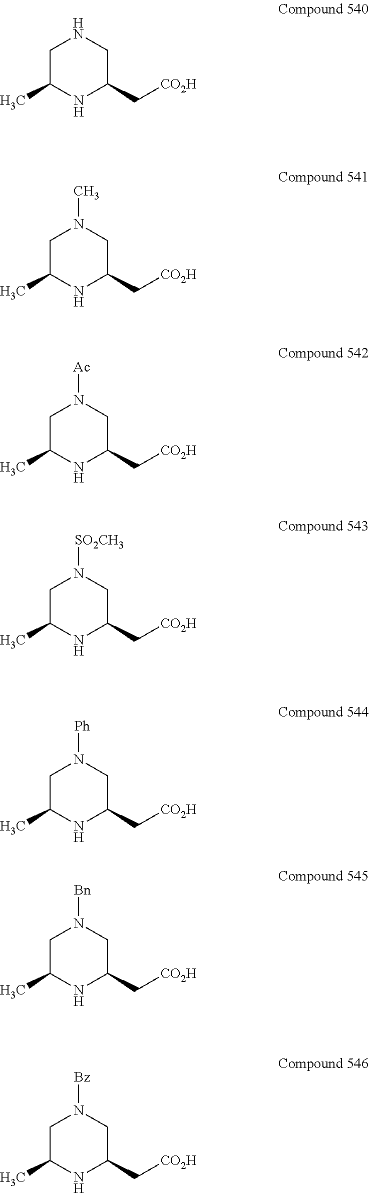

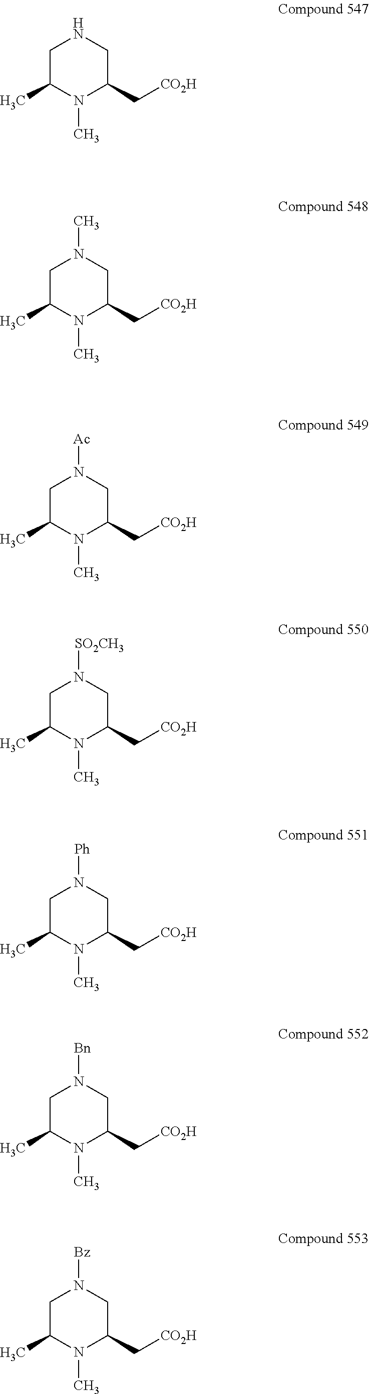

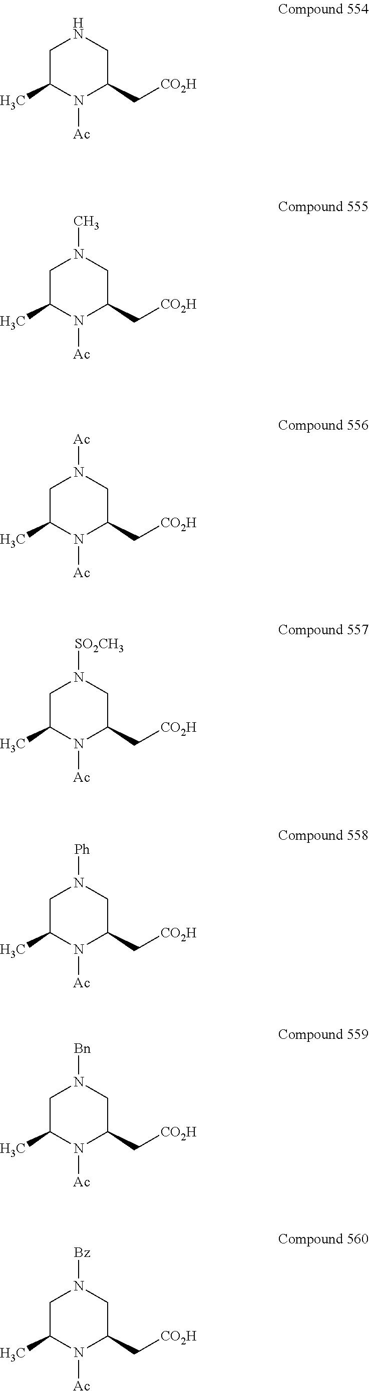

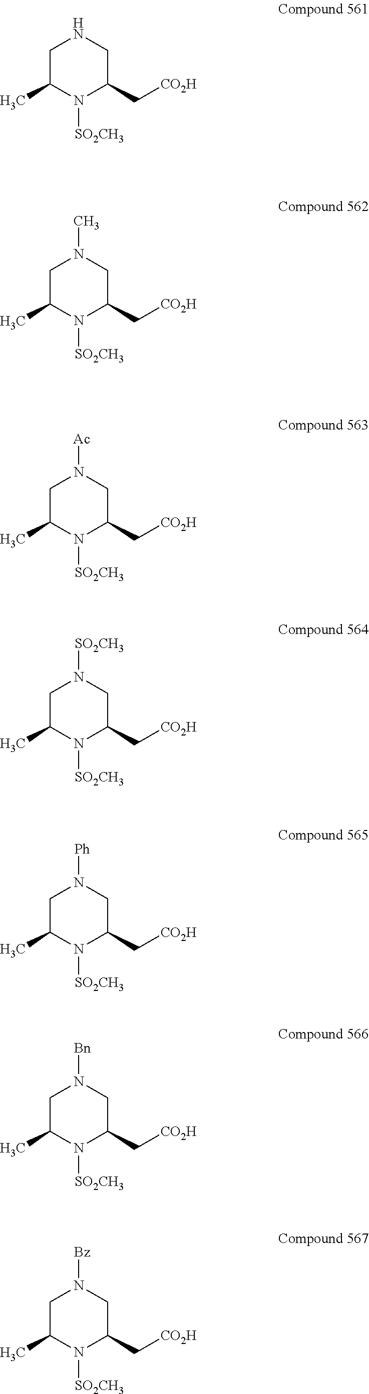

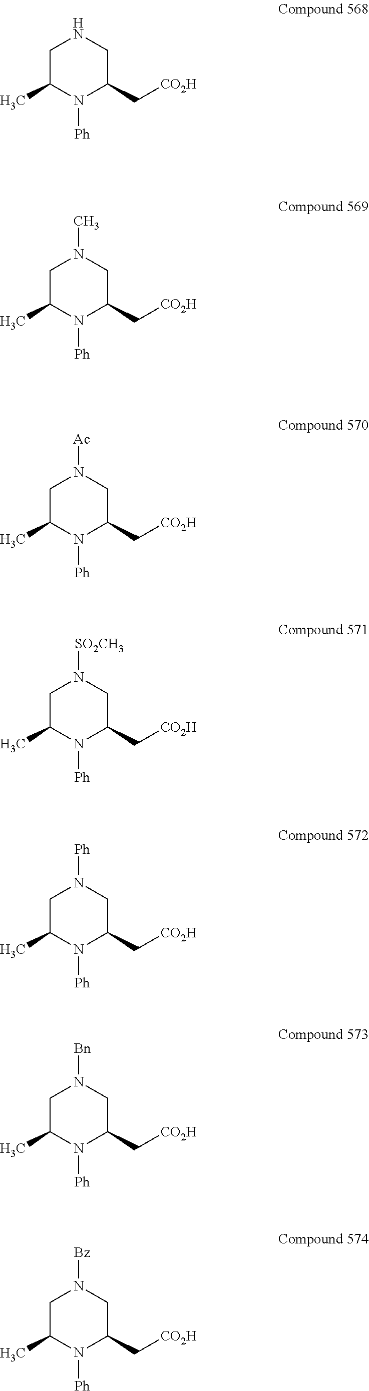

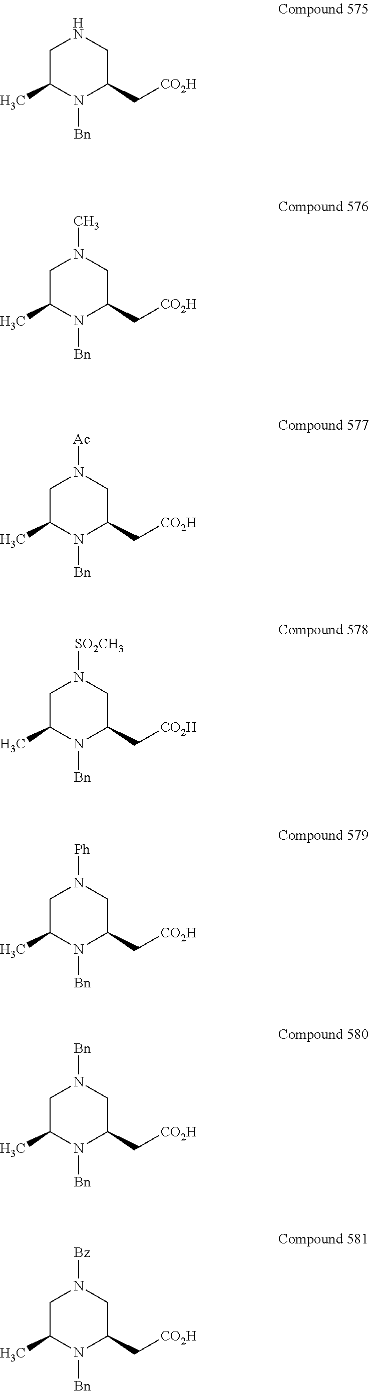

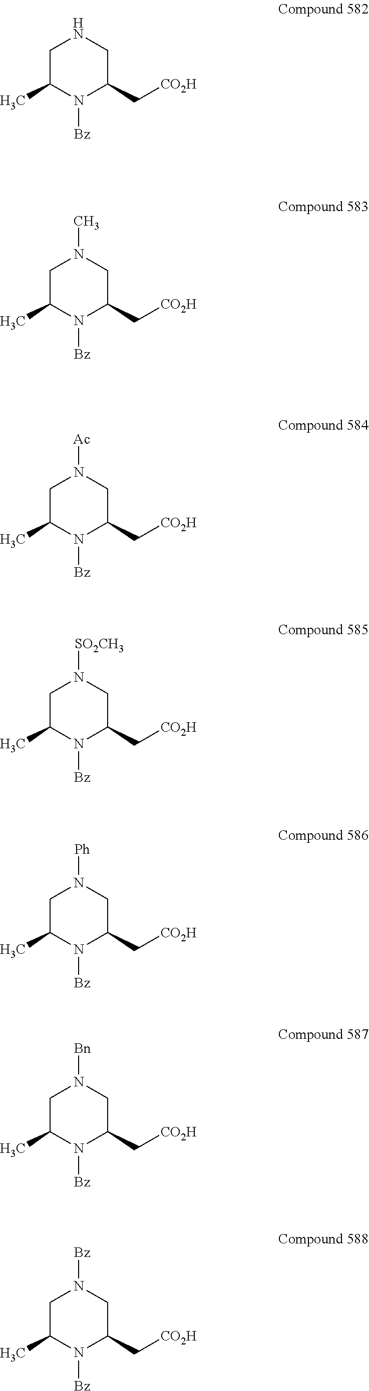

In some cases, the compounds according to Structure I-A are represented by Structure I-A13:

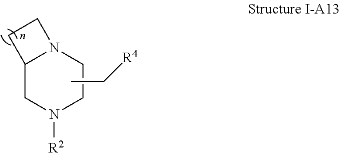

In Structure I-A13, R² and R⁴ are as defined above for Formula I. Also in Structure I-A13, n is 0, 1, 2, or 3.

In some cases, the compounds according to Structure I-A are represented by Structure I-A14:

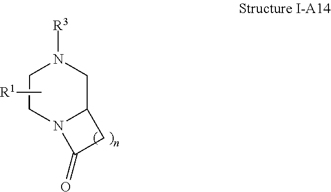

In Structure I-A14, R¹ and R³ are as defined above for Formula I. Also in Structure I-A14, n is 1, 2, or 3.

In some cases, the compounds according to Structure I-A are represented by Structure I-A15:

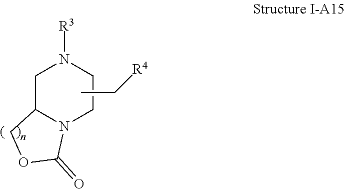

In Structure I-A15, R³ and R⁴ are as defined above for Formula I. Also in Structure I-A15, n is 1 or 2.

In some cases, the compounds according to Structure I-A are represented by Structure I-A16:

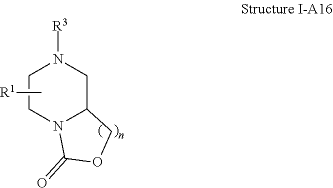

In Structure I-A16, R¹ and R³ are as defined above for Formula I. Also in Structure I-A16, n is 1 or 2.

In some cases, the compounds according to Formula I are represented by Structure I-B:

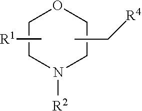

and pharmaceutically acceptable salts or prodrugs thereof.

In Structure I-B, R¹ is substituted or unsubstituted alkyl, substituted or unsubstituted heteroalkyl, substituted or unsubstituted aryl, substituted or unsubstituted heteroaryl.

Also in Structure I-B, R² is hydrogen, substituted or unsubstituted alkyl, substituted or unsubstituted carbonyl, substituted or unsubstituted sulfonyl, substituted or unsubstituted aryl, or substituted or unsubstituted heteroaryl.

Additionally in Structure I-B, R⁴ is substituted or unsubstituted alkyl, substituted or unsubstituted amino, hydroxyl, substituted or unsubstituted alkoxy, substituted or unsubstituted carbonyl, or substituted or unsubstituted sulfonyl.

Optionally in Structure I-B, R¹ and R² or R² and R⁴ can combine to form a substituted or unsubstituted heterocycloalkyl or a substituted or unsubstituted heteroaryl.

In some cases, the compounds according to Formula I are represented by Structure I-C:

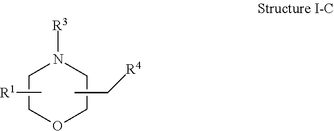

and pharmaceutically acceptable salts or prodrugs thereof.

In Structure I-C, R¹ is substituted or unsubstituted alkyl, substituted or unsubstituted heteroalkyl, substituted or unsubstituted aryl, or substituted or unsubstituted heteroaryl.

Also in Structure I-C, R³ is hydrogen, substituted or unsubstituted alkyl, substituted or unsubstituted carbonyl, substituted or unsubstituted sulfonyl, substituted or unsubstituted aryl, or substituted or unsubstituted heteroaryl.

Additionally in Structure I-C, R⁴ is substituted or unsubstituted alkyl, substituted or unsubstituted amino, hydroxyl, substituted or unsubstituted alkoxy, substituted or unsubstituted carbonyl, or substituted or unsubstituted sulfonyl.

Optionally in Structure I-C, R¹ and R³ or R³ and R⁴ can combine to form a substituted or unsubstituted heterocycloalkyl or a substituted or unsubstituted heteroaryl.

Also described herein are compounds represented by Formula II:

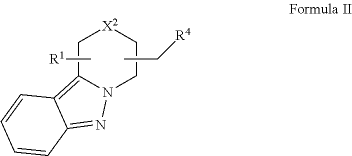

and pharmaceutically acceptable salts or prodrugs thereof.

In Formula II, X² is NR² or O.

Also in Formula II, R¹ is substituted or unsubstituted alkyl, substituted or unsubstituted heteroalkyl, substituted or unsubstituted aryl, or substituted or unsubstituted heteroaryl.

Additionally in Formula II, R⁴ is substituted or unsubstituted alkyl, substituted or unsubstituted amino, hydroxyl, substituted or unsubstituted alkoxy, substituted or unsubstituted carbonyl, or substituted or unsubstituted sulfonyl.

In some cases, the compounds according to Formula II are represented by Structure II-A:

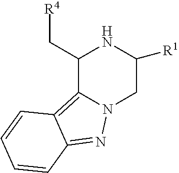

In Structure II-A, R¹ and R⁴ are as defined above for Formula II.

In some cases, the compounds according to Formula II are represented by Structure II-B:

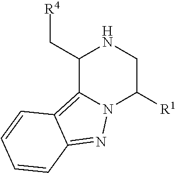

In Structure II-B, R¹ and R⁴ are as defined above for Formula II.

In some cases, the compounds according to Formula II are represented by Structure II-C:

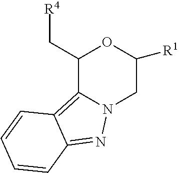

In Structure II-C, R¹ and R⁴ are as defined above for Formula II.

In some cases, the compounds according to Formula II are represented by Structure II-D:

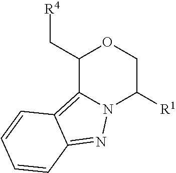

In Structure II-D, R¹ and R⁴ are as defined above for Formula II.

Further described herein are compounds represented by Formula III:

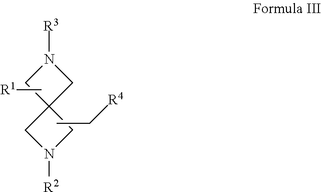

and pharmaceutically acceptable salts or prodrugs thereof.

In Formula III, R¹ is substituted or unsubstituted alkyl, substituted or unsubstituted heteroalkyl, substituted or unsubstituted aryl, or substituted or unsubstituted heteroaryl.

Also in Formula III, R² and R³ are independently selected from the group consisting of hydrogen, substituted or unsubstituted alkyl, substituted or unsubstituted carbonyl, substituted or unsubstituted sulfonyl, substituted or unsubstituted aryl, or substituted or unsubstituted heteroaryl.

Additionally in Formula III, R⁴ is substituted or unsubstituted alkyl, substituted or unsubstituted amino, hydroxyl, substituted or unsubstituted alkoxy, substituted or unsubstituted carbonyl, or substituted or unsubstituted sulfonyl.

In Formula III, the R¹ and R⁴ substituents can be located on either ring of the compound.

As used herein, the terms alkyl, alkenyl, and alkynyl include straight- and branched-chain monovalent substituents. Examples include methyl, ethyl, isobutyl, 3-butynyl, and the like. Ranges of these groups useful with the compounds and methods described herein include C₁-C₂₀ alkyl, C₂-C₂₀ alkenyl, and C₂-C₂₀ alkynyl. Additional ranges of these groups useful with the compounds and methods described herein include C₁-C₁₂ alkyl, C₂-C₁₂ alkenyl, C₂-C₁₂ alkynyl, C₁-C₆ alkyl, C₂-C₆ alkenyl, C₂-C₆ alkynyl, C₁-C₄ alkyl, C₂-C₄ alkenyl, and C₂-C₄ alkynyl.

Heteroalkyl, heteroalkenyl, and heteroalkynyl are defined similarly as alkyl, alkenyl, and alkynyl, but can contain O, S, or N heteroatoms or combinations thereof within the backbone. Ranges of these groups useful with the compounds and methods described herein include C₁-C₂₀ heteroalkyl, C₂-C₂₀ heteroalkenyl, and C₂-C₂₀ heteroalkynyl. Additional ranges of these groups useful with the compounds and methods described herein include C₁-C₁₂ heteroalkyl, C₂-C₁₂ heteroalkenyl, C₂-C₁₂ heteroalkynyl, C₁-C₆ heteroalkyl, C₂-C₆ heteroalkenyl, C₂-C₆ heteroalkynyl, C₁-C₄ heteroalkyl, C₂-C₄ heteroalkenyl, and C₂-C₄ heteroalkynyl.

The terms cycloalkyl, cycloalkenyl, and cycloalkynyl include cyclic alkyl groups having a single cyclic ring or multiple condensed rings. Examples include cyclohexyl, cyclopentylethyl, and adamantanyl. Ranges of these groups useful with the compounds and methods described herein include C₃-C₂₀ cycloalkyl, C₃-C₂₀ cycloalkenyl, and C₃-C₂₀ cycloalkynyl. Additional ranges of these groups useful with the compounds and methods described herein include C₅-C₁₂ cycloalkyl, C₅-C₁₂ cycloalkenyl, C₅-C₁₂ cycloalkynyl, C₅-C₆ cycloalkyl, C₅-C₆ cycloalkenyl, and C₅-C₆ cycloalkynyl.

The terms heterocycloalkyl, heterocycloalkenyl, and heterocycloalkynyl are defined similarly as cycloalkyl, cycloalkenyl, and cycloalkynyl, but can contain O, S, or N heteroatoms or combinations thereof within the cyclic backbone. Ranges of these groups useful with the compounds and methods described herein include C₃-C₂₀ heterocycloalkyl, C₃-C₂₀ heterocycloalkenyl, and C₃-C₂₀ heterocycloalkynyl. Additional ranges of these groups useful with the compounds and methods described herein include C₅-C₁₂ heterocycloalkyl, C₅-C₁₂ heterocycloalkenyl, C₅-C₁₂ heterocycloalkynyl, C₅-C₆ heterocycloalkyl, C₅-C₆ heterocycloalkenyl, and C₅-C₆ heterocycloalkynyl.

Aryl molecules include, for example, cyclic hydrocarbons that incorporate one or more planar sets of, typically, six carbon atoms that are connected by delocalized electrons numbering the same as if they consisted of alternating single and double covalent bonds. An example of an aryl molecule is benzene. Heteroaryl molecules include substitutions along their main cyclic chain of atoms such as O, N, or S. When heteroatoms are introduced, a set of five atoms, e.g., four carbon and a heteroatom, can create an aromatic system. Examples of heteroaryl molecules include furan, pyrrole, thiophene, imadazole, oxazole, pyridine, and pyrazine. Aryl and heteroaryl molecules can also include additional fused rings, for example, benzofuran, indole, benzothiophene, naphthalene, anthracene, and quinoline. The aryl and heteroaryl molecules can be attached at any position on the ring, unless otherwise noted.

The term alkoxy as used herein is an alkyl group bound through a single, terminal ether linkage. The term aryloxy as used herein is an aryl group bound through a single, terminal ether linkage. Likewise, the terms alkenyloxy, alkynyloxy, heteroalkyloxy, heteroalkenyloxy, heteroalkynyloxy, heteroaryloxy, cycloalkyloxy, and heterocycloalkyloxy as used herein are an alkenyloxy, alkynyloxy, heteroalkyloxy, heteroalkenyloxy, heteroalkynyloxy, heteroaryloxy, cycloalkyloxy, and heterocycloalkyloxy group, respectively, bound through a single, terminal ether linkage.

The term hydroxy as used herein is represented by the formula —OH.

The terms amine or amino as used herein are represented by the formula —NZ¹Z², where Z¹ and Z² can each be substitution group as described herein, such as hydrogen, an alkyl, halogenated alkyl, alkenyl, alkynyl, aryl, heteroaryl, cycloalkyl, cycloalkenyl, heterocycloalkyl, or heterocycloalkenyl group described above.

The alkoxy, cycloalkoxy, aryloxy, amino, alkyl, alkenyl, alkynyl, aryl, heteroalkyl, heteroalkenyl, heteroalkynyl, heteroaryl, cycloalkyl, or heterocycloalkyl molecules used herein can be substituted or unsubstituted. As used herein, the term substituted includes the addition of an alkoxy, cycloalkoxy, aryloxy, amino, alkyl, alkenyl, alkynyl, aryl, heteroalkyl, heteroalkenyl, heteroalkynyl, heteroaryl, cycloalkyl, or heterocycloalkyl group to a position attached to the main chain of the alkoxy, cycloalkoxy, aryloxy, amino, alkyl, alkenyl, alkynyl, aryl, heteroalkyl, heteroalkenyl, heteroalkynyl, heteroaryl, cycloalkyl, or heterocycloalkyl, e.g., the replacement of a hydrogen by one of these molecules. Examples of substitution groups include, but are not limited to, hydroxy, halogen (e.g., F, Br, Cl, or I), and carboxyl groups. Conversely, as used herein, the term unsubstituted indicates the alkoxy, cycloalkoxy, aryloxy, amino, alkyl, alkenyl, alkynyl, aryl, heteroalkyl, heteroalkenyl, heteroalkynyl, heteroaryl, cycloalkyl, or heterocycloalkyl has a full complement of hydrogens, i.e., commensurate with its saturation level, with no substitutions, e.g., linear decane (—(CH₂)₉—CH₃).

### I. Methods of Making the Compounds

The compounds described herein can be prepared in a variety of ways. The compounds can be synthesized using various synthetic methods. At least some of these methods are known in the art of synthetic organic chemistry. The compounds described herein can be prepared from readily available starting materials. Optimum reaction conditions can vary with the particular reactants or solvent used, but such conditions can be determined by one skilled in the art by routine optimization procedures.

Variations on Formula I, Formula II, and Formula III include the addition, subtraction, or movement of the various constituents as described for each compound. Similarly, when one or more chiral centers are present in a molecule, all possible chiral variants are included. Additionally, compound synthesis can involve the protection and deprotection of various chemical groups. The use of protection and deprotection, and the selection of appropriate protecting groups can be determined by one skilled in the art. The chemistry of protecting groups can be found, for example, in Wuts, Greene's Protective Groups in Organic Synthesis, 5th. Ed., Wiley & Sons, 2014, which is incorporated herein by reference in its entirety.

Reactions to produce the compounds described herein can be carried out in solvents, which can be selected by one of skill in the art of organic synthesis. Solvents can be substantially nonreactive with the starting materials (reactants), the intermediates, or products under the conditions at which the reactions are carried out, i.e., temperature and pressure. Reactions can be carried out in one solvent or a mixture of more than one solvent. Product or intermediate formation can be monitored according to any suitable method known in the art. For example, product formation can be monitored by spectroscopic means, such as nuclear magnetic resonance spectroscopy (e.g., ¹H or ¹³C) infrared spectroscopy, spectrophotometry (e.g., UV-visible), or mass spectrometry, or by chromatography such as high performance liquid chromatography (HPLC) or thin layer chromatography.

Reductive methylation was used to introduce the N-methyl capping group. Exposure of the piperazine scaffolds described herein bearing an unprotected N₁ or N₄ atom to 37% aq. HCHO for a short period of time (for example, less than <30 minutes at room temperature) followed by treatment with sodium triacetoxyborohydride (STAB)/AcOH quantitatively converted them to N-methylated derivatives. Acetonitrile (CAN) can be used as the solvent for the reaction.

Acetylation and benzoylation of piperazines bearing an unprotected nitrogen atom at either N₁ or N₄ can be accomplished by reacting with AcCl or PhC(O)Cl (BzCl) in the presence an amine (e.g., trimethylamine) and a solvent or solvent mixture (e.g., DCM) at room temperature to afford N-acetyl/benzoyl derivatives (Scheme 1).

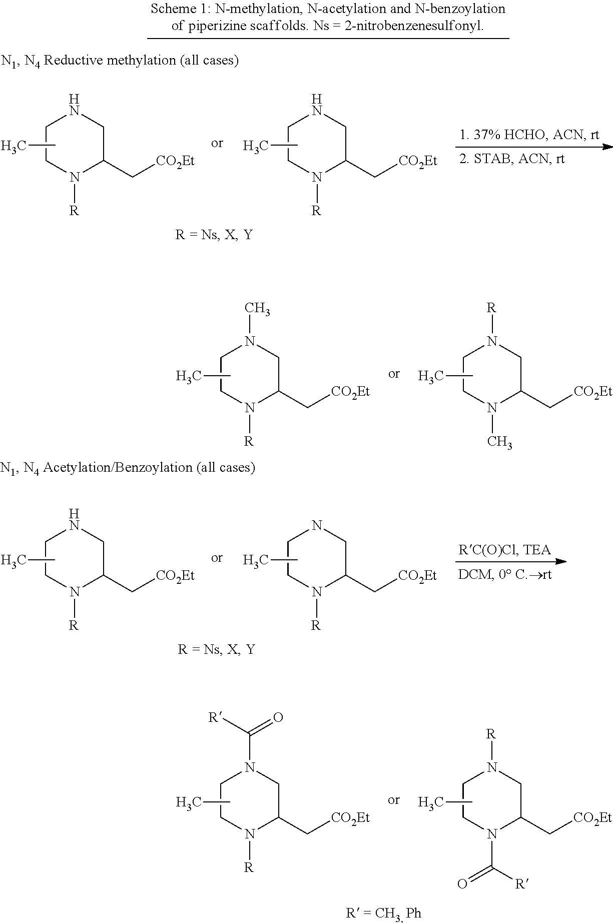

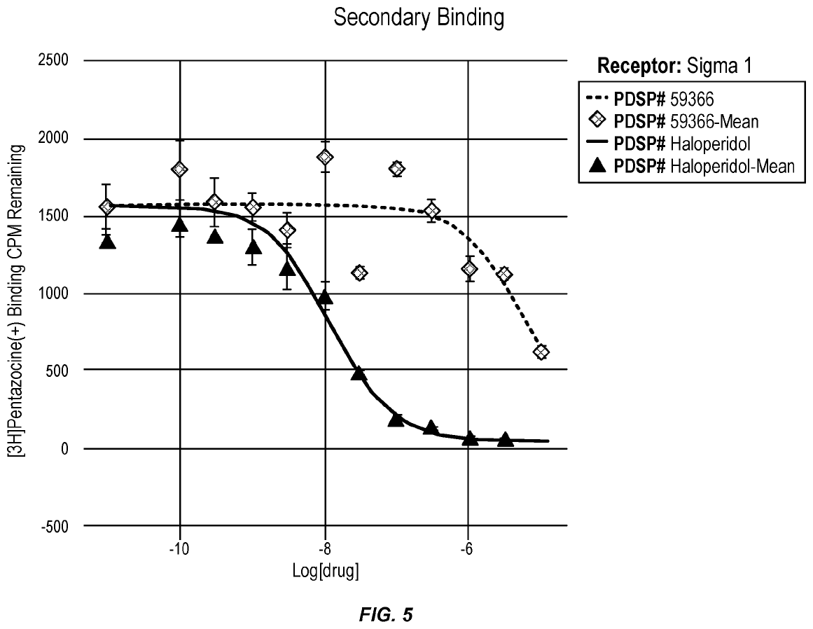

N-mesylation methodology (MsCl, TEA, DCM, room temperature) can be used to convert all unprotected nitrogen atoms in both the 2,3- and 2,5-scaffold family branches to N-mesyl derivatives (FIG. 5). The N₄ atom of the 2,6-scaffold family branch is also efficiently converted to its N₄-mesyl derivative using the same method. The N₁ atom of the 2,6-scaffold family branch, being the most sterically hindered, is more resistant. The trans diastereomers can be converted to N₁-mesyl derivatives by treatment with MsCl/pyr (for example, at 60° C., 3 h).

A de novo batch synthesis of the cis N₁-mesyl 2,6-scaffold derivatives was devised as shown in Scheme 2. The antipodes of alaninol are converted to their bis-methanesulfonamides. Reaction with NaN₃ affords the methanesulfonamide azides. Staudinger reduction produces the monoprotected diamine, which is converted to its bis-sulfonaminde derivative and then regioselectively alkylated with ethyl 4-bromocrotonate to afford the cyclization precursor (e.g., Bordwell pKa (DMSO) MeSO₂NH₂=17.5; PhSO₂NH₂=16.1). Base promoted cyclization affords a mixture of cis and trans diastereomers (dr=2.3:1). The diastereomers are separated by column chromatography, giving the N₁-mesyl-N₄-Ns piperazines. Denosylation provides the cis N₁-mesyl 2,6-scaffolds with an unprotected N₄ atom available for diversification.

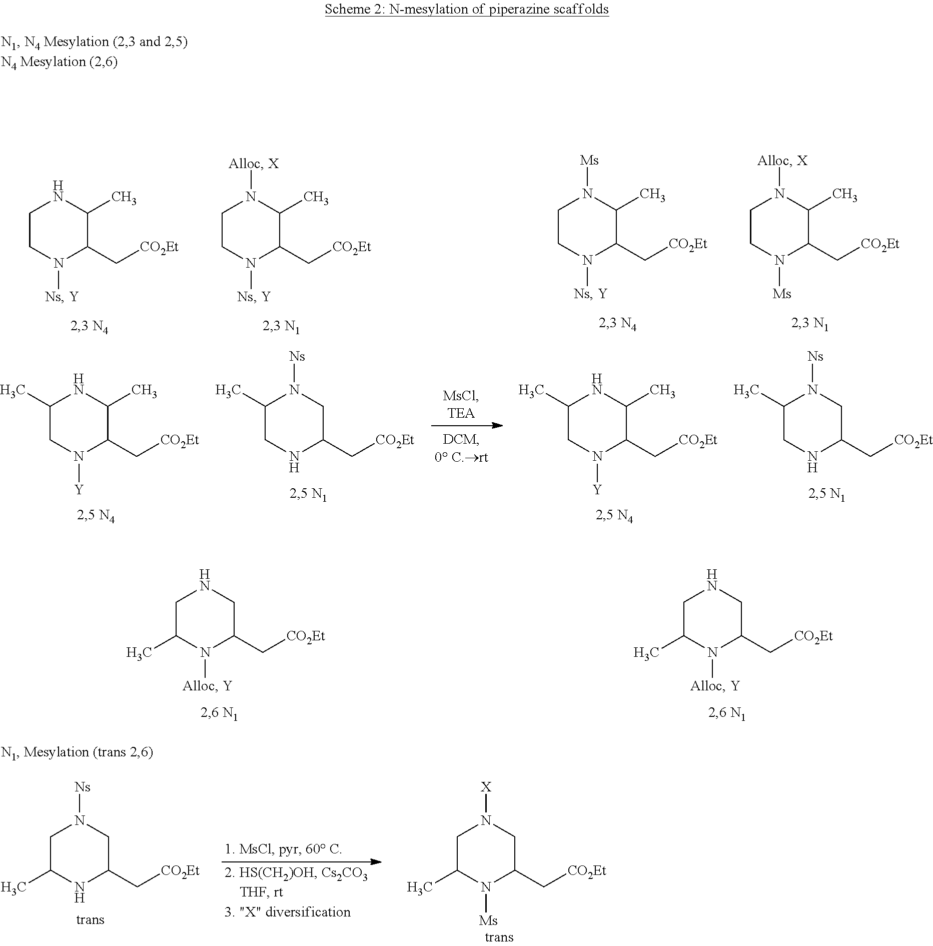

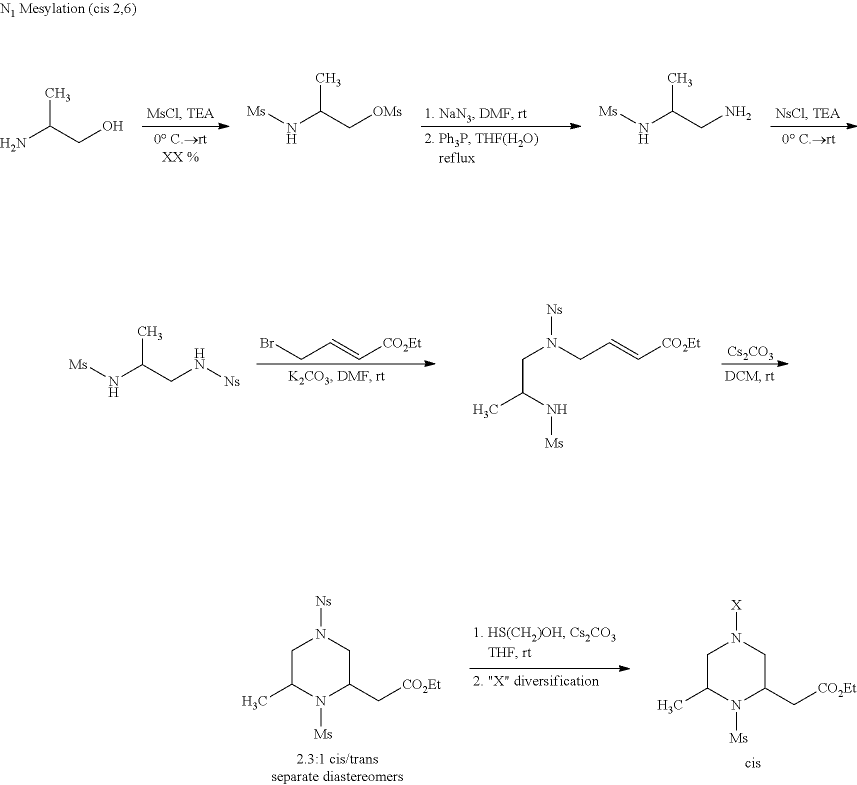

Benzylation follows a similar pattern as mesylation (Scheme 3). All unprotected nitrogen atoms in the 2,3- and 2,5-scaffold family branches, as well as the N₄ atom of the 2,6-branch, are efficiently benzylated using reductive alkylation conditions (for example, PhCHO, AcOH, STAB, DCM, room temperature). For the 2,6-branch, reductive alkylation fails to fully convert the N₁ atom of both diastereomers to their benzylated derivatives. Instead, alkylation (e.g., PhCH₂Br, K₂CO₃, DMF, TBAI (cat), room temperature) affords the desired N-benzyl derivatives with no evidence of quaternization.

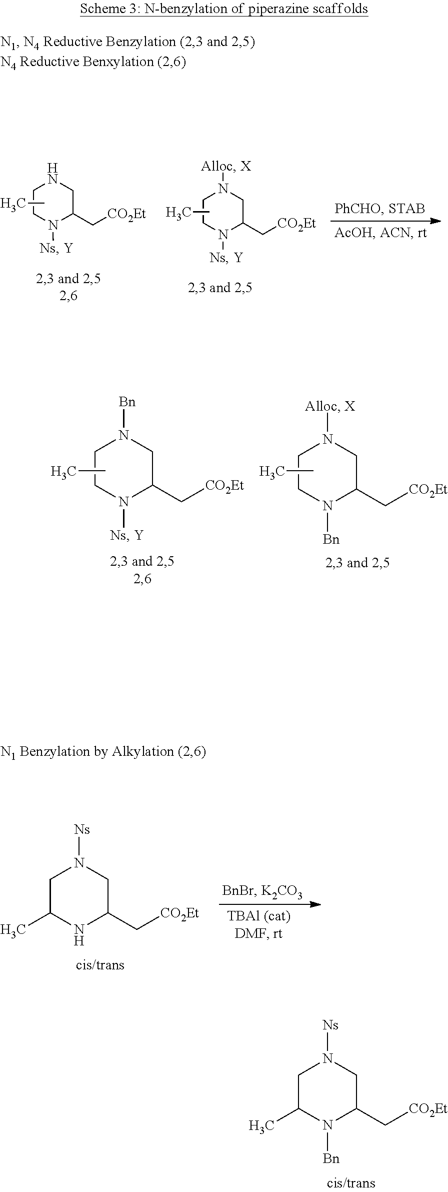

N-Phenylation can be performed according to the synthetic method shown in Scheme 4. N-phenylation was the use of benzyne chemistry. Using ((2-trifluoromethylsulfonyloxy))phenyl)trimethylsilane as the benzyne source, a number of fluoride reagents can be used, including tetrabutylammonium fluoride (TBAF) or tetrabutylammonium triflate (TBAT).

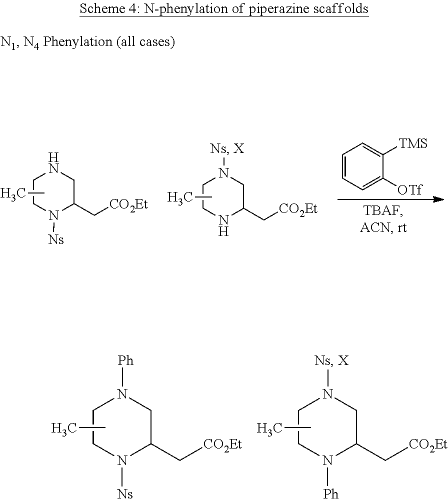

In cases where the target compound requires protection of the free N atom that emerged from the scaffold synthesis prior to diversification, the Alloc group is found to effectively protect either N₁ or N₄ in all twelve stereoisomers. The production sequence is shown in Scheme 5. Alloc protection was followed by denosylation. The denosylated N atom is diversified and the Alloc group removed using (TPP)₄Pd with excess di-isopropylamine (DIA) added as a scavenger for the allyl cation byproduct.

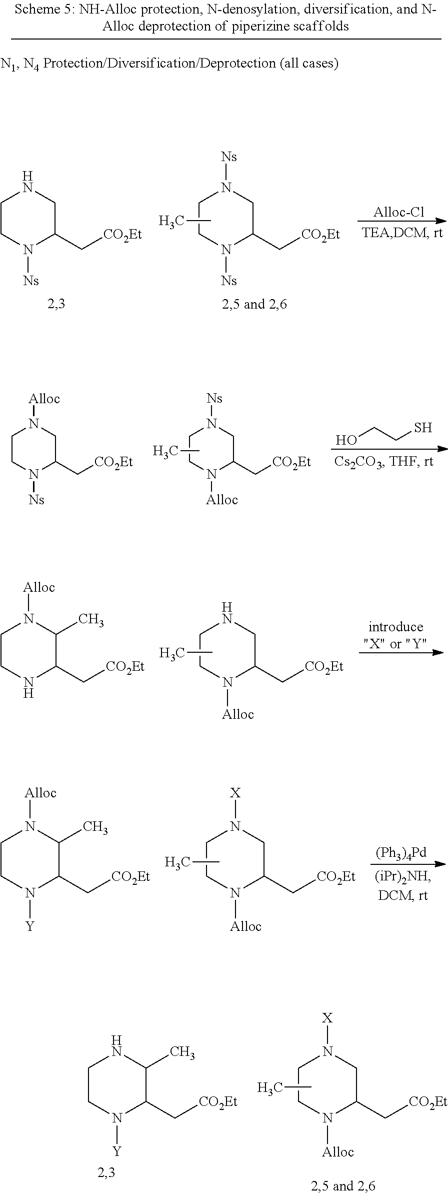

Exemplary procedures for synthesizing the compounds as described herein are provided in Example 1 below.

### III. Pharmaceutical Formulations

The compounds described herein or derivatives thereof can be provided in a pharmaceutical composition. Depending on the intended mode of administration, the pharmaceutical composition can be in the form of solid, semi-solid or liquid dosage forms, such as, for example, tablets, suppositories, pills, capsules, powders, liquids, or suspensions, preferably in unit dosage form suitable for single administration of a precise dosage. The compositions will include a therapeutically effective amount of the compound described herein or derivatives thereof in combination with a pharmaceutically acceptable carrier and, in addition, may include other medicinal agents, pharmaceutical agents, carriers, or diluents. By pharmaceutically acceptable is meant a material that is not biologically or otherwise undesirable, which can be administered to an individual along with the selected compound without causing unacceptable biological effects or interacting in a deleterious manner with the other components of the pharmaceutical composition in which it is contained.

As used herein, the term carrier encompasses any excipient, diluent, filler, salt, buffer, stabilizer, solubilizer, lipid, stabilizer, or other material well known in the art for use in pharmaceutical formulations. The choice of a carrier for use in a composition will depend upon the intended route of administration for the composition. The preparation of pharmaceutically acceptable carriers and formulations containing these materials is described in, e.g., Remington: The Science and Practice of Pharmacy, 22d Edition, Loyd et al. eds., Pharmaceutical Press and Philadelphia College of Pharmacy at University of the Sciences (2012). Examples of physiologically acceptable carriers include buffers, such as phosphate buffers, citrate buffer, and buffers with other organic acids; antioxidants including ascorbic acid; low molecular weight (less than about 10 residues) polypeptides; proteins, such as serum albumin, gelatin, or immunoglobulins; hydrophilic polymers, such as polyvinylpyrrolidone; amino acids such as glycine, glutamine, asparagine, arginine or lysine; monosaccharides, disaccharides, and other carbohydrates, including glucose, mannose, or dextrins; chelating agents, such as EDTA; sugar alcohols, such as mannitol or sorbitol; salt-forming counterions, such as sodium; and/or nonionic surfactants, such as TWEEN® (ICI, Inc.; Bridgewater, New Jersey), polyethylene glycol (PEG), and PLURONICS™ (BASF; Florham Park, NJ).

Compositions containing the compound described herein or derivatives thereof suitable for parenteral injection may comprise physiologically acceptable sterile aqueous or nonaqueous solutions, dispersions, suspensions or emulsions, and sterile powders for reconstitution into sterile injectable solutions or dispersions. Examples of suitable aqueous and nonaqueous carriers, diluents, solvents or vehicles include water, ethanol, polyols (propyleneglycol, polyethyleneglycol, glycerol, and the like), suitable mixtures thereof, vegetable oils (such as olive oil) and injectable organic esters such as ethyl oleate. Proper fluidity can be maintained, for example, by the use of a coating such as lecithin, by the maintenance of the required particle size in the case of dispersions and by the use of surfactants.

These compositions may also contain adjuvants, such as preserving, wetting, emulsifying, and dispensing agents. Prevention of the action of microorganisms can be promoted by various antibacterial and antifungal agents, for example, parabens, chlorobutanol, phenol, sorbic acid, and the like. Isotonic agents, for example, sugars, sodium chloride, and the like may also be included. Prolonged absorption of the injectable pharmaceutical form can be brought about by the use of agents delaying absorption, for example, aluminum monostearate and gelatin.

Solid dosage forms for oral administration of the compounds described herein or derivatives thereof include capsules, tablets, pills, powders, and granules. In such solid dosage forms, the compounds described herein or derivatives thereof is admixed with at least one inert customary excipient (or carrier), such as sodium citrate or dicalcium phosphate, or (a) fillers or extenders, as for example, starches, lactose, sucrose, glucose, mannitol, and silicic acid, (b) binders, as for example, carboxymethylcellulose, alignates, gelatin, polyvinylpyrrolidone, sucrose, and acacia, (c) humectants, as for example, glycerol, (d) disintegrating agents, as for example, agar-agar, calcium carbonate, potato or tapioca starch, alginic acid, certain complex silicates, and sodium carbonate, (e) solution retarders, as for example, paraffin, (f) absorption accelerators, as for example, quaternary ammonium compounds, (g) wetting agents, as for example, cetyl alcohol, and glycerol monostearate, (h) adsorbents, as for example, kaolin and bentonite, and (i) lubricants, as for example, talc, calcium stearate, magnesium stearate, solid polyethylene glycols, sodium lauryl sulfate, or mixtures thereof. In the case of capsules, tablets, and pills, the dosage forms may also comprise buffering agents.

Solid compositions of a similar type may also be employed as fillers in soft and hard-filled gelatin capsules using such excipients as lactose or milk sugar as well as high molecular weight polyethyleneglycols, and the like.

Solid dosage forms such as tablets, dragees, capsules, pills, and granules can be prepared with coatings and shells, such as enteric coatings and others known in the art. They may contain opacifying agents and can also be of such composition that they release the active compound or compounds in a certain part of the intestinal tract in a delayed manner. Examples of embedding compositions that can be used are polymeric substances and waxes. The active compounds can also be in micro-encapsulated form, if appropriate, with one or more of the above-mentioned excipients.

Liquid dosage forms for oral administration of the compounds described herein or derivatives thereof include pharmaceutically acceptable emulsions, solutions, suspensions, syrups, and elixirs. In addition to the active compounds, the liquid dosage forms may contain inert diluents commonly used in the art, such as water or other solvents, solubilizing agents, and emulsifiers, as for example, ethyl alcohol, isopropyl alcohol, ethyl carbonate, ethyl acetate, benzyl alcohol, benzyl benzoate, propyleneglycol, 1,3-butyleneglycol, dimethylformamide, oils, in particular, cottonseed oil, groundnut oil, corn germ oil, olive oil, castor oil, sesame oil, glycerol, tetrahydrofurfuryl alcohol, polyethyleneglycols, and fatty acid esters of sorbitan, or mixtures of these substances, and the like.

Besides such inert diluents, the composition can also include additional agents, such as wetting, emulsifying, suspending, sweetening, flavoring, or perfuming agents.

Suspensions, in addition to the active compounds, may contain additional agents, as for example, ethoxylated isostearyl alcohols, polyoxyethylene sorbitol and sorbitan esters, microcrystalline cellulose, aluminum metahydroxide, bentonite, agar-agar and tragacanth, or mixtures of these substances, and the like.

Compositions of the compounds described herein or derivatives thereof for rectal administrations are optionally suppositories, which can be prepared by mixing the compounds with suitable non-irritating excipients or carriers, such as cocoa butter, polyethyleneglycol or a suppository wax, which are solid at ordinary temperatures but liquid at body temperature and, therefore, melt in the rectum or vaginal cavity and release the active component.

Dosage forms for topical administration of the compounds described herein or derivatives thereof include ointments, powders, sprays, inhalants, and skin patches. The compounds described herein or derivatives thereof are admixed under sterile conditions with a physiologically acceptable carrier and any preservatives, buffers, or propellants as may be required. Ophthalmic formulations, ointments, powders, and solutions are also contemplated as being within the scope of the compositions.

Optionally, the compounds described herein can be contained in a drug depot. A drug depot comprises a physical structure to facilitate implantation and retention in a desired site (e.g., a synovial joint, a disc space, a spinal canal, abdominal area, a tissue of the patient, etc.). The drug depot can provide an optimal concentration gradient of the compound at a distance of up to about 0.1 cm to about 5 cm from the implant site. A depot, as used herein, includes but is not limited to capsules, microspheres, microparticles, microcapsules, microfibers particles, nanospheres, nanoparticles, coating, matrices, wafers, pills, pellets, emulsions, liposomes, micelles, gels, antibody-compound conjugates, protein-compound conjugates, or other pharmaceutical delivery compositions. Suitable materials for the depot include pharmaceutically acceptable biodegradable materials that are preferably FDA approved or GRAS materials. These materials can be polymeric or non-polymeric, as well as synthetic or naturally occurring, or a combination thereof. The depot can optionally include a drug pump.

The compositions can include one or more of the compounds described herein and a pharmaceutically acceptable carrier. As used herein, the term pharmaceutically acceptable salt refers to those salts of the compound described herein or derivatives thereof that are, within the scope of sound medical judgment, suitable for use in contact with the tissues of subjects without undue toxicity, irritation, allergic response, and the like, commensurate with a reasonable benefit/risk ratio, and effective for their intended use, as well as the zwitterionic forms, where possible, of the compounds described herein. The term salts refers to the relatively non-toxic, inorganic and organic acid addition salts of the compounds described herein. These salts can be prepared in situ during the isolation and purification of the compounds or by separately reacting the purified compound in its free base form with a suitable organic or inorganic acid and isolating the salt thus formed. Representative salts include the hydrobromide, hydrochloride, sulfate, bisulfate, nitrate, acetate, oxalate, valerate, oleate, palmitate, stearate, laurate, borate, benzoate, lactate, phosphate, tosylate, citrate, maleate, fumarate, succinate, tartrate, naphthylate mesylate, glucoheptonate, lactobionate, methane sulphonate, and laurylsulphonate salts, and the like. These may include cations based on the alkali and alkaline earth metals, such as sodium, lithium, potassium, calcium, magnesium, and the like, as well as non-toxic ammonium, quaternary ammonium, and amine cations including, but not limited to ammonium, tetramethylammonium, tetraethylammonium, methylamine, dimethylamine, trimethylamine, triethylamine, ethylamine, and the like. (See S. M. Barge et al., J. Pharm. Sci. (1977) 66, 1, which is incorporated herein by reference in its entirety, at least, for compositions taught therein.)

Administration of the compounds and compositions described herein or pharmaceutically acceptable salts thereof can be carried out using therapeutically effective amounts of the compounds and compositions described herein or pharmaceutically acceptable salts thereof as described herein for periods of time effective to treat a disorder. The effective amount of the compounds and compositions described herein or pharmaceutically acceptable salts thereof as described herein may be determined by one of ordinary skill in the art and includes exemplary dosage amounts for a mammal of from about 0.0001 to about 200 mg/kg of body weight of active compound per day, which may be administered in a single dose or in the form of individual divided doses, such as from 1 to 4 times per day. Alternatively, the dosage amount can be from about 0.01 to about 150 mg/kg of body weight of active compound per day, about 0.1 to 100 mg/kg of body weight of active compound per day, about 0.5 to about 75 mg/kg of body weight of active compound per day, about 0.5 to about 50 mg/kg of body weight of active compound per day, about 0.01 to about 50 mg/kg of body weight of active compound per day, about 0.05 to about 25 mg/kg of body weight of active compound per day, about 0.1 to about 25 mg/kg of body weight of active compound per day, about 0.5 to about 25 mg/kg of body weight of active compound per day, about 1 to about 20 mg/kg of body weight of active compound per day, about 1 to about 10 mg/kg of body weight of active compound per day, about 20 mg/kg of body weight of active compound per day, about 10 mg/kg of body weight of active compound per day, about 5 mg/kg of body weight of active compound per day, about 2.5 mg/kg of body weight of active compound per day, about 1.0 mg/kg of body weight of active compound per day, or about 0.5 mg/kg of body weight of active compound per day, or any range derivable therein. Optionally, the dosage amounts are from about 0.01 mg/kg to about 10 mg/kg of body weight of active compound per day. Optionally, the dosage amount is from about 0.01 mg/kg to about 5 mg/kg. Optionally, the dosage amount is from about 0.01 mg/kg to about 2.5 mg/kg.

Those of skill in the art will understand that the specific dose level and frequency of dosage for any particular subject may be varied and will depend upon a variety of factors, including the activity of the specific compound employed, the metabolic stability and length of action of that compound, the species, age, body weight, general health, sex and diet of the subject, the mode and time of administration, rate of excretion, drug combination, and severity of the particular condition.

The precise dose to be employed in the formulation will also depend on the route of administration, and the seriousness of the disease or disorder, and should be decided according to the judgment of the practitioner and each subject's circumstances. Effective doses can be extrapolated from dose-response curves derived from in vitro or animal model test systems. Further, depending on the route of administration, one of skill in the art would know how to determine doses that result in a plasma concentration for a desired level of response in the cells, tissues and/or organs of a subject.

### IV. Methods of Use

Provided herein are methods to treat or prevent a condition or disease associated with a G protein-coupled receptor (GPCR) in a subject. The methods include administering to a subject an effective amount of one or more of the compounds or compositions described herein, or a pharmaceutically acceptable salt or prodrug thereof. Effective amount, when used to describe an amount of compound in a method, refers to the amount of a compound that achieves the desired pharmacological effect or other biological effect. The effective amount can be, for example, the concentrations of compounds at which a targeted GPCR is modulated in vitro, as provided herein. Also contemplated is a method that includes administering to the subject an amount of one or more compounds described herein such that an in vivo concentration at a target cell in the subject corresponding to the concentration administered in vitro is achieved.

The compounds and compositions described herein or pharmaceutically acceptable salts thereof are useful for treating GPCR-associated conditions or diseases in humans, including, without limitation, pediatric and geriatric populations, and in animals, e.g., veterinary applications.

In some examples, the GPCR-associated condition or disease is ageing or an age-related disease. Optionally, the age-related disease is insulin resistance (also referred to as impaired glucose tolerance), benign prostatic hyperplasia, hearing loss, osteoporosis, age-related macular degeneration, neurodegenerative diseases such as Alzheimer's disease or Parkinson's disease, a skin disease, or aging skin.

In some examples, the GPCR-associated condition or disease is cancer. Optionally, the cancer is bladder cancer, brain cancer, breast cancer (e.g., triple negative breast cancer), bronchus cancer, colorectal cancer (e.g., colon cancer, rectal cancer), cervical cancer, chondrosarcoma, endometrial cancer, gastrointestinal cancer, gastric cancer, genitourinary cancer, glioblastoma, head and neck cancer, hepatic cancer, hepatocellular carcinoma, leukemia, liver cancer, lung cancer, lymphoma, melanoma of the skin, ovarian cancer, pancreatic cancer, prostate cancer, renal cancer, skin cancer, testicular cancer, thyroid cancer, or uterine cancer. Optionally, the cancer is a cancer that affects one or more of the following sites: oral cavity and pharynx (e.g., tongue, mouth, pharynx, or other oral cavity); digestive system (e.g., esophagus, stomach, small intestine, colon, rectum, anus, anal canal, anorectum, liver and intrahepatic bile duct, gallbladder and other biliary, pancreas, or other digestive organs); respiratory system (e.g., larynx, lung and bronchus, or other respiratory organs); bones and joints; soft tissue (e.g., heart); skin (e.g., melanoma of the skin or other nonepithelial skin); breast; genital system (e.g., uterine cervix, uterine corpus, ovary, vulva, vagina and other female genital areas, prostate, testis, penis and other male genital areas); urinary system (e.g., urinary bladder, kidney and renal pelvis, and ureter and other urinary organs); eye and orbit; brain and other nervous system; endocrine system (e.g., thyroid and other endocrine); lymphoma (e.g., Hodgkin lymphoma and non-Hodgkin lymphoma); myeloma; or leukemia (e.g., acute lymphocytic leukemia, chronic lymphocytic leukemia, acute myeloid leukemia, chronic myeloid leukemia, or other leukemia).

In some examples, the GPCR-associated condition or disease is a cardiovascular disorder, such as atherosclerosis, post-angioplasty, restenosis, coronary artery diseases or angina.

In some examples, the GPCR-associated condition or disease is a hematologic disorder. Optionally, the hematologic disorder can be a leukemia (e.g., acute myeloid leukemia, acute promyelocytic leukemia, acute lymphoblastic leukemia, chronic myelogenous leukemia), the myelodysplastic syndromes, or an anemia (e.g., sickle cell anemia).

In some examples, the GPCR-associated condition or disease is an infectious disease. Optionally, the infectious disease can result from the presence of pathogenic agents, including pathogenic viruses, pathogenic bacteria, fungi, protozoa, multicellular parasites and aberrant proteins known as prions. Optionally, the infectious disease can include virus infectious diseases and bacterial infectious diseases. The virus infectious disease is not particularly limited and includes, for example, infectious diseases with respiratory infectious viruses (e.g., infectious diseases due to respiratory infectious viruses such as influenza virus, rhino virus, corona virus, parainfluenza virus, RS virus, adeno virus, reo virus and the like), herpes zoster caused by herpes virus, diarrhea caused by rotavirus, viral hepatitis, AIDS and the like. The bacterial infectious disease is not particularly limited and includes, for example, infectious diseases caused by Bacillus cereus, Vibrio parahaemolyticus, Enterohemorrhagic Escherichia coli, Staphylococcus aureus (e.g., methicillin-resistant Staphylococcus aureus), Salmonella, Botulinus, Candida and the like.

In some examples, the GPCR-associated condition or disease is an inflammatory disease. Optionally, the inflammatory disorder can be respiratory or pulmonary disorders (including asthma, COPD, chronic bronchitis and cystic fibrosis); inflammatory diseases of the joints (including rheumatoid and osteoarthritis); skin disorders (including dermatitis, eczematous dermatitis and psoriasis); post transplantation late and chronic solid organ rejection; multiple sclerosis; autoimmune conditions (including systemic lupus erythematosus, dermatomyositis, polymyositis, Sjogren's syndrome, polymyalgia rheumatica, temporal arteritis, Behcet's disease, Guillain Barre, Wegener's granulomatosus, polyarteritis nodosa); inflammatory neuropathies (including inflammatory polyneuropathies); vasculitis (including Churg-Strauss syndrome, Takayasu's arteritis); inflammatory disorders of adipose tissue; and proliferative disorders (including Kaposi's sarcoma and other proliferative disorders of smooth muscle cells).

In some examples, the GPCR-associated condition or disease is a metabolic disease. Optionally, the metabolic disease is obesity, diabetes, or a genetic disorder.

In some examples, the GPCR-associated condition or disease is a neurodegenerative disorder. Optionally, the neurodegenerative disorder is Alexander disease, Alper's disease, Alzheimer disease, amyotrophic lateral sclerosis, ataxia telangiectasia, Batten disease (also known as Spielmeyer-Vogt-Sjogren-Batten disease), Canavan disease, Cockayne syndrome, Corticobasal degeneration, Creutzfeldt-Jakob disease, Huntington's disease, Kennedy's disease, Krabbe disease, Lewy body dementia, Machado-Joseph disease, Spinocerebellar ataxia type 3, multiple sclerosis, multiple system atrophy, Parkinson's disease, Pelizaeus-Merzbacher disease, Pick's disease, Primary lateral sclerosis, Refsum's disease, Sandhoff disease, Schilder's disease, Spielmeyer-Vogt-Sjogren-Batten disease (also known as Batten disease), Spinocerebellar ataxia (multiple types with varying characteristics), Spinal muscular atrophy, Steele-Richardson-Olszewski disease, Tay-Sachs, Transmissible spongiform encephalopathies (TSE), or Tabes dorsalis.

In some examples, the GPCR-associated condition or disease is a respiratory disease, such as chronic obstructive pulmonary disease, chronic bronchitis, emphysema, or asthma.

In some examples, the GPCR-associated condition or disease is a urological disorder. Optionally, the urological disorder can be urinary incontinence, overflow incontinence, stress incontinence, idiopathic chronic urinary retention, interstitial cystitis, neuro-urological disorder, vesico-urethral dysfunctions, bladder inflammation, bladder pain, pelvic pain, constipation, and genito-urinary disorders such as prostatitis, prostatalgia, or prostatodynia.

The methods of treating or preventing a GPCR-associated condition or disease in a subject can further comprise administering to the subject a second compound, biomolecule, or composition. The one or more additional agents and the compounds described herein or pharmaceutically acceptable salts or prodrugs thereof can be administered in any order, including concomitant, simultaneous, or sequential administration. Sequential administration can be administration in a temporally spaced order of up to several days apart. The methods can also include more than a single administration of the one or more additional agents and the compounds described herein or pharmaceutically acceptable salts or prodrugs thereof. The administration of the one or more additional agents and the compounds described herein or pharmaceutically acceptable salts or prodrugs thereof can be by the same or different routes and concurrently or sequentially.

Any of the aforementioned therapeutic agents can be used in any combination with the compositions described herein. Combinations are administered either concomitantly (e.g., as an admixture), separately but simultaneously (e.g., via separate intravenous lines into the same subject), or sequentially (e.g., one of the compounds or agents is given first followed by the second). Thus, the term combination is used to refer to concomitant, simultaneous, or sequential administration of two or more agents.

The methods and compounds as described herein are useful for both prophylactic and therapeutic treatment. For prophylactic use, a therapeutically effective amount of the compounds and compositions or pharmaceutically acceptable salts thereof as described herein are administered to a subject prior to onset (e.g., before obvious signs of a GPCR-associated condition or disease), during early onset (e.g., upon initial signs and symptoms of a GPCR-associated condition or disease), or after the development of a GPCR-associated condition or disease. Prophylactic administration can occur for several days to years prior to the manifestation of symptoms of a GPCR-associated condition or disease. Therapeutic treatment involves administering to a subject a therapeutically effective amount of the compounds and compositions or pharmaceutically acceptable salts thereof as described herein after a GPCR-associated condition or disease is diagnosed.

The methods of treating or preventing a GPCR-associated condition or disease can also include administering the compounds or pharmaceutical compositions described herein by one or more clinically acceptable routes. The compounds or pharmaceutical compositions described herein can be administered orally, intraperitoneally, sublingually, subcutaneously, intravenously, or any clinically acceptable administration route.

The compounds described herein are also useful in regulating a G protein-coupled receptor in a cell. The methods for regulating a G protein-coupled receptor in a cell include contacting a cell with an effective amount of one or more of the compounds as described herein. Optionally, the contacting is performed in vivo. Optionally, the contacting is performed in vitro.

Optionally, the G protein-coupled receptor is a serotonin receptor (e.g., 5-HT1A, 5-HT1B, 5-HT1D, 5-HT1E, 5-HT2A, 5-HT2B, 5-HT2C, 5-HT3, 5-HT5A, 5-HT6, and 5-HT7A), a dopamine receptor (e.g., D1, D2, D3, D4, and D5), a muscarinic receptor (e.g., M1, M2, M3, M4, and M5), an adrenergic receptor (e.g., Alpha-1A, Alpha-1B, Alpha-1C, Alpha-1D, Alpha-2A, Alpha-2B, Alpha-2C, Beta-1, Beta-2, and Beta-3), a histamine receptor (e.g., H1, H2, H3, and H4), or a sigma receptor (e.g., Sigma-1 and Sigma-2). Optionally, the G protein-coupled receptor is selected from the group consisting of kappa-opioid receptor (KOR), mu-opioid receptor (MOR), delta-opioid receptor (DOR), gamma-aminobutyric acid A (GABAA) receptor, peripheral-type benzodiazepine receptors (PBR), dopamine transporter (DAT) receptor, norepinephrine transporter (NET) receptor, serotonin transporter (SERT) receptor, and benzodiazepine (BZP) rat brain receptors.

### V. Kits

Also provided herein are kits for treating or preventing a condition or disease associated with a G protein-coupled receptor (e.g., ageing, cancer, a cardiovascular disorder, a hematologic disorder, an infectious disease, an inflammatory disease, a metabolic disease, a neurodegenerative disorder, a respiratory disease, and/or a urological disorder) in a subject. A kit can include any of the compounds or compositions described herein. For example, a kit can include one or more compounds of Formula I, Formula II, and/or Formula III. A kit can further include one or more additional agents, such as a second compound, biomolecule, or composition (e.g., one or more anti-inflammatory agents or anti-cancer agents. A kit can include an oral formulation of any of the compounds or compositions described herein. A kit can include an intravenous formulation of any of the compounds or compositions described herein. A kit can additionally include directions for use of the kit (e.g., instructions for treating a subject), a container, a means for administering the compounds or compositions (e.g., a syringe), and/or a carrier.

As used herein the terms treatment, treat, or treating refer to a method of reducing one or more symptoms of a disease or condition. Thus in the disclosed method, treatment can refer to a 10%, 20%, 30%, 40%, 50%, 60%, 70%, 80%, 90%, or 100% reduction in the severity of one or more symptoms of the disease or condition. For example, a method for treating a disease is considered to be a treatment if there is a 10% reduction in one or more symptoms or signs (e.g., size of a tumor or rate of tumor growth) of the disease in a subject as compared to a control. As used herein, control refers to the untreated condition (e.g., at-risk populations not treated with the compounds and compositions described herein or tumor cells not treated with the compounds and compositions described herein). Thus the reduction can be a 10%, 20%, 30%, 40%, 50%, 60%, 70%, 80%, 90%, 100%, or any percent reduction in between 10% and 100% as compared to native or control levels. It is understood that treatment does not necessarily refer to a cure or complete ablation of the disease, condition, or symptoms of the disease or condition.

As used herein, the terms prevent, preventing, and prevention of a disease or disorder refer to an action, for example, administration of a composition or therapeutic agent, that occurs before or at about the same time a subject begins to show one or more symptoms of the disease or disorder, which inhibits or delays onset or severity of one or more symptoms of the disease or disorder.

As used herein, references to decreasing, reducing, or inhibiting include a change of 10%, 20%, 30%, 40%, 50%, 60%, 70%, 80%, 90% or greater as compared to a control level. Such terms can include, but do not necessarily include, complete elimination.

As used herein, subject means both mammals and non-mammals. Mammals include, for example, humans; non-human primates, e.g., apes and monkeys; cattle; horses; sheep; rats; mice; pigs; and goats. Non-mammals include, for example, fish and birds.

Throughout this application, various publications are referenced. The disclosures of these publications in their entireties are hereby incorporated by reference into this application.

The examples below are intended to further illustrate certain aspects of the methods and compositions described herein, and are not intended to limit the scope of the claims.

### EXAMPLES

### Example 1: Compound Synthesis

The compounds described herein were prepared according to Schemes 1-5 shown above using the general procedures outlined below.

Reductive Methylation. A 10 mL glass reaction vessel with a filter frit and male Luer tip was placed into the rubber female Luer receptacle of a Bohdan MiniBlock. The MiniBlock was placed atop a Bohdan MiniBlock orbital shaker. The vessel was charged with a solution of secondary amine (1 eq) in dry ACN (2 mL). A septum sheet and bracket was placed atop the block and secured to the vessel by clamping; 37% formaldehyde (10.0 eq) was added by syringe followed by acetic acid (1 equiv), also added by syringe. The shaker was turned on and set to a rate that ensured thorough mixing (˜600 rpm). Agitation was continued for 30 minutes. Agitation was halted, and the septum sheet was removed. Sodium triacetoxyborohydride (3 equiv) was added. The septum sheet was re-secured atop the reactor; agitation was resumed and continued for 16 hours. UPLC/MS analysis indicated the complete conversion to methylated product. The MiniBlock was removed from the shaker and placed atop a Bohdan MiniBlock filtration extension block having a glass tube positioned directly below the reaction vessel. A valve in the top block was opened allowing the contents of the reaction vessel to drain through the filter frit, the Luer tip, and into the glass tube below. The homogeneous solution was evaporated to a residue. The residue was redissolved in DCM (2 mL) and returned to another glass reaction vessel in a block atop the shaker. Saturated aqueous NaHCO₃ (2 mL) was added by pipet. The septum sheet was secured atop the reaction vessel and agitation carried out for 5 min. Agitation was stopped, and the 2-phase mixture was allowed to drain through a phase separator. The lower organic layer passed through the separator membrane into a glass tube, while the upper aqueous was retained in the separator. The organic was evaporated to a residue and carried forward into the next reaction without further purification.

Acetylation/Mesylation. A 10 mL glass vessel was positioned as described above. The vessel was charged with a solution of secondary amine/amine HCl salt (based on complete conversion to its corresponding deprotected derivative) in dry DCM (2 mL). The vessel was sealed with a septum sheet. Triethylamine (2 equivalents) was added by syringe. Slow agitation (˜100 rpm) was initiated, and acetyl chloride (for acetylation)/methanesulfonyl chloride (for mesylation) (2 equivalents) was slowly added by syringe. Normal agitation (600 rpm) was initiated and continued for 5 h. UPLC/MS indicated complete conversion to product. Saturated aqueous NaHCO₃ (2 mL) was added, and the 2-phase mixture was agitated for 10 minutes. Agitation was stopped, and the mixture drained into a phase separator. The lower organic layer was recovered in a glass tube and evaporated to a residue. The residue was carried forward into the next reaction without further purification.

Nosyl Deprotection. A 10 mL glass vessel was positioned as described above. The vessel was charged with a solution of nosyl compound (based on complete conversion of previous reaction) in dry THF (2 mL). 2-Mercaptoethanol (3 equiv) was added by syringe, followed by Cs₂CO₃ (2 equiv). The vessel was sealed with a septum sheet and agitated for 16 hours. UPLC/MS analysis indicated complete removal of the 2-nosyl protecting group. Agitation was stopped; the reaction mixture was drained into a glass tube and evaporated to a residue. The residue was dissolved in DCM (2 mL) and transferred to a new 10 mL glass vessel in a block atop the shaker. Aqueous HCl (2 N, 2 mL) was added. The vessel was sealed with a septum sheet and agitated for 5 minutes. The 2-phase mixture was drained into a phase separator. The retained aqueous layer was recovered and evaporated to give a residue containing the HCl salt of the deprotected amine. The material was carried on to the next reaction without further purification

Reductive Benzylation. A 10 mL glass vessel was positioned as described above. The vessel was charged with a solution of secondary amine (1 eq) in dry DCM (2 mL), benzaldehyde (2.0 eq) was added by syringe followed by acetic acid (0.5 equiv), also added by syringe. The shaker was turned on and set to a rate that ensured thorough mixing (˜600 rpm). Agitation was continued for 30 minutes. Agitation was halted, and the septum sheet was removed. Sodium triacetoxyborohydride (2 equiv) was added. The septum sheet was re-secured atop the reactor; agitation was resumed and continued for 16 hours. UPLC/MS analysis indicated the complete conversion to benzylated product. Saturated aqueous NaHCO₃ (2 mL) was added, and the 2-phase mixture was agitated for 10 minutes. Agitation was stopped, and the mixture drained into a phase separator. The lower organic layer was recovered in a glass tube and evaporated to a residue. The residue was carried forward into the next reaction without further purification.

Phenylation. A 10 mL glass vessel was positioned as described above. The vessel was charged with a solution of secondary amine (1 eq) in dry ACN (2 mL) and ((2-trifluoromethylsulfonyloxy))phenyl)trimethylsilane (2.0 eq) was added by syringe followed by 1M solution of TBAF (2.0 eq). The shaker turned on and normal agitation (600 rpm) was initiated and continued for 5 hours. UPLC/MS indicated complete conversion to product. Agitation was stopped; the reaction mixture was drained into a glass tube and evaporated to a residue. The residue was dissolved in DCM (2 mL) and passed through a pad of silica to eliminate excess TBAF salt. The collected organic layer was transferred into a glass tube and evaporated to a residue. The residue was carried forward into the next reaction without further purification.

Hydrolysis. A 10 mL glass vessel was positioned as described above. The vessel was charged with a solution of ethyl ester compound (based on complete conversion of its corresponding bis-diversified derivative) in THF (2 mL). The vessel was sealed with a septum sheet. Aqueous 1 M LiOH·H₂O solution (2 equiv) was added by syringe. Normal agitation (600 rpm) was initiated and continued for 6 hours. UPLC/MS indicated complete conversion to carboxylic acid. Aqueous 1 M HCl (1 mL) was added, and the mixture was agitated for 10 minutes. Agitation was stopped, and the mixture was drained into a glass tube. The glass tube was evaporated to a residue. The residue was triturated with absolute EtOH (2×2 mL). The EtOH supernatant was recovered and evaporated to yield final compound.

### Example 2: Activity Data

Compounds as described herein were tested for activity against a host of GPCR receptors, including adrenergic receptors (Alpha-1A, Alpha-1B, Alpha-1D, Alpha-2A, Alpha-2B, Alpha-2C, Beta-1, Beta-2, and Beta-3); dopamine receptors (D1, D2, D3, D4, and D5); histamine receptors (H1, H2, H3, and H4); muscarinic acetylcholine receptors (M1, M2, M3, M4, and M5); neurotransmitter transporters (DAT, NET, and SERT); opioid receptors (DOR, KOR, and MOR); serotonin receptors (5-HT1A, 5-HT1B, 5-HT1D, 5-HT1E, 5-HT2A, 5-HT2B, 5-HT2C, 5-HT3, 5-HT5A, 5-HT6, and 5-HT7A); and other receptors, including BZP rat brain site, GABAA, PBR, Sigma 1, and Sigma 2). The tested compounds are shown below, along with the compound labeling used for the assays:

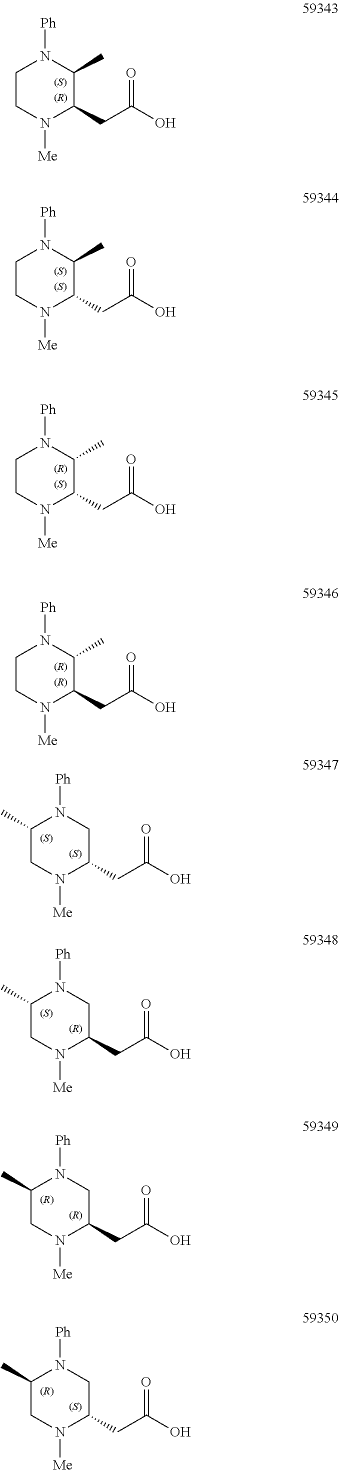

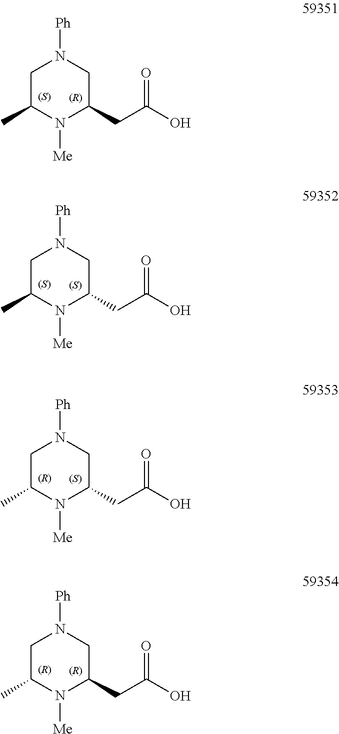

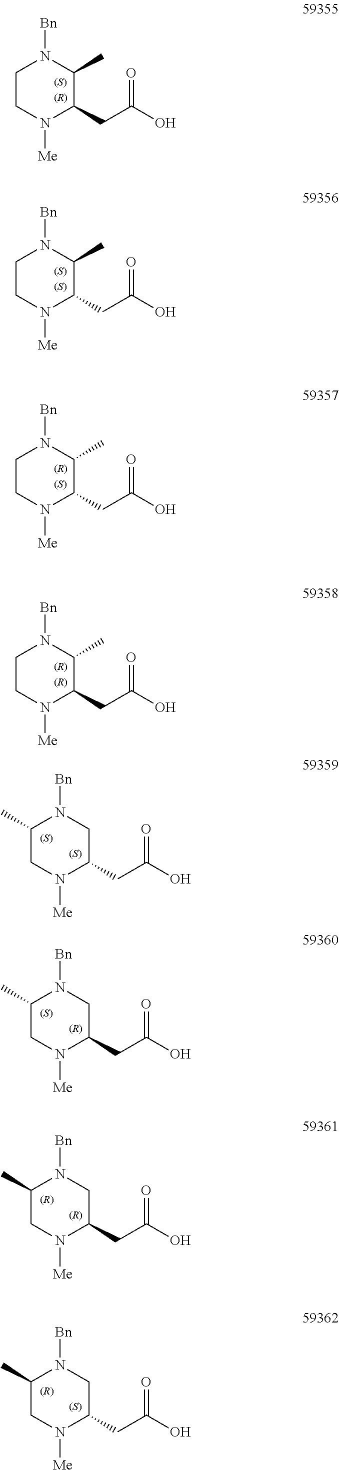

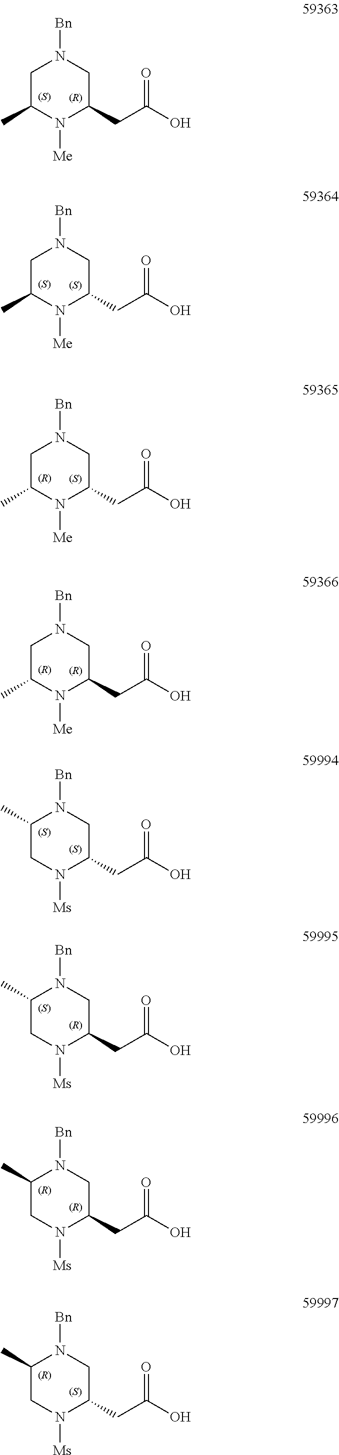

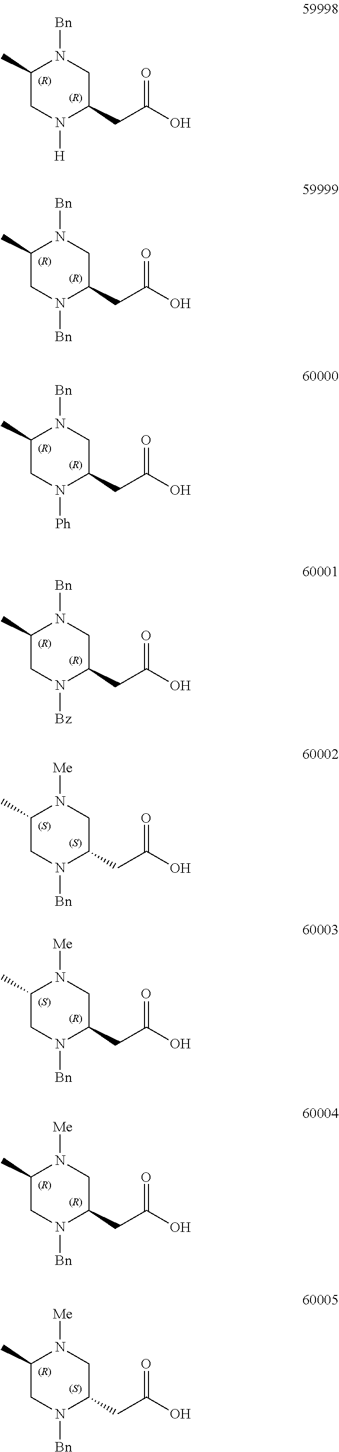

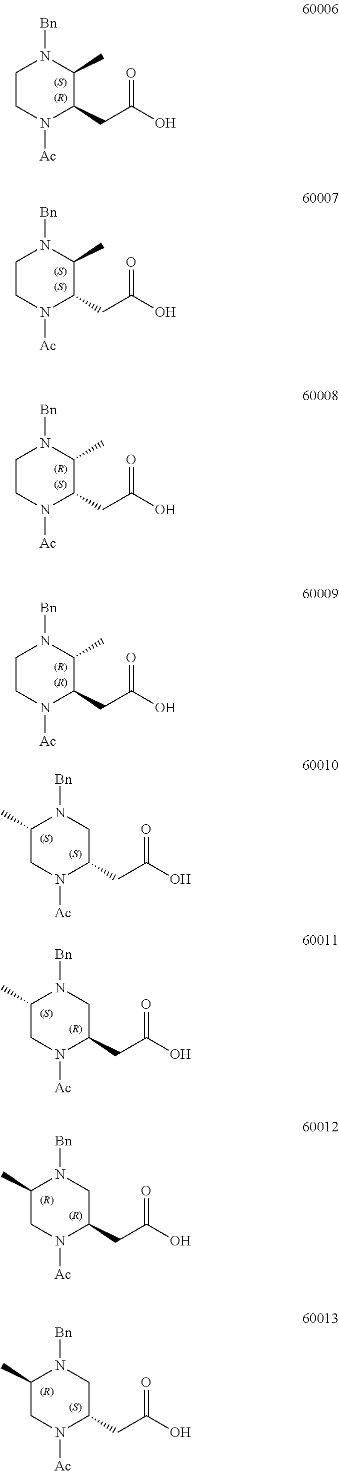

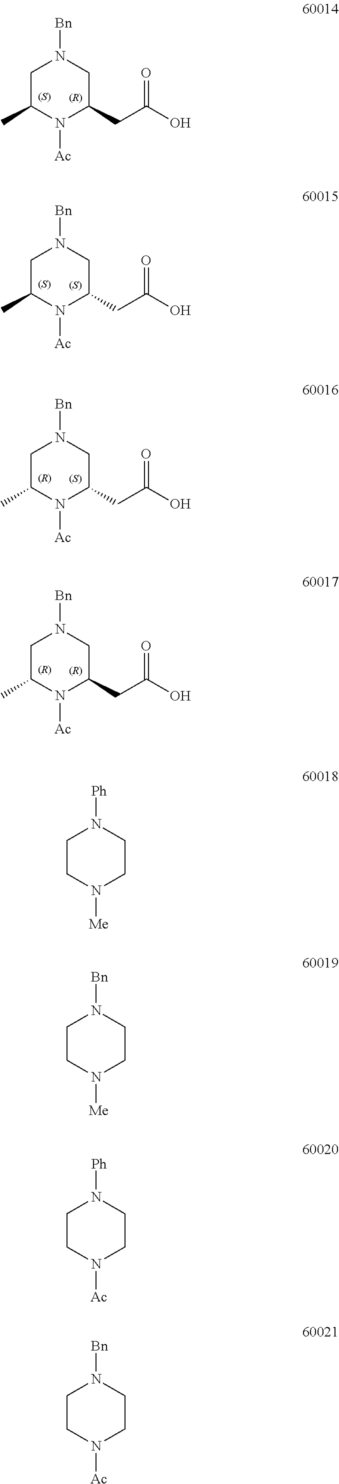

Primary and secondary radioligand binding assays were performed. Both primary and secondary radioligand binding assays were carried out in a final of volume of 125 μl per well in appropriate binding buffers. The hot ligand concentration was at a concentration close to the Kd. Total binding and nonspecific binding were determined in the absence and presence of 10 μM of the appropriate reference compound, respectively. In brief, plates were incubated at room temperature and in the dark for 90 minutes. Reactions were stopped by vacuum filtration onto 0.3% polyethyleneimine (PEI) soaked 96-well filter mats using a 96-well Filtermate harvester, followed by three washes with cold wash buffers. Scintillation cocktail is then melted onto the microwave-dried filters on a hot plate and radioactivity is counted in a Microbeta counter.

A general stepwise procedure for the binding assays that was used is shown below:
(1) Prepare appropriate binding buffers and wash buffers;
(2) Prepare 2.5× of final concentration of radioligand working solution;
(3) Count 50 μl of radioligand working solution to confirm radioligand concentration and activity;
(4) Add 50 μl radioligand to each well;
(5) Add 50 μl membrane suspension to each well;
(6) Mix by gentle and brief shaking;
(7) Incubate the plates in the dark for desired time (usually 60-90 min at RT);
(8) Soak filters in cold 0.3% PEI;
(9) Stop the reaction by vacuum filtration and washing;
(10) Dry the filters using microwave oven;
(11) Melt scintillation cocktail on top of filters;
(12) Wrap filters in plastic wrap; and
(13) Count radioactivity.

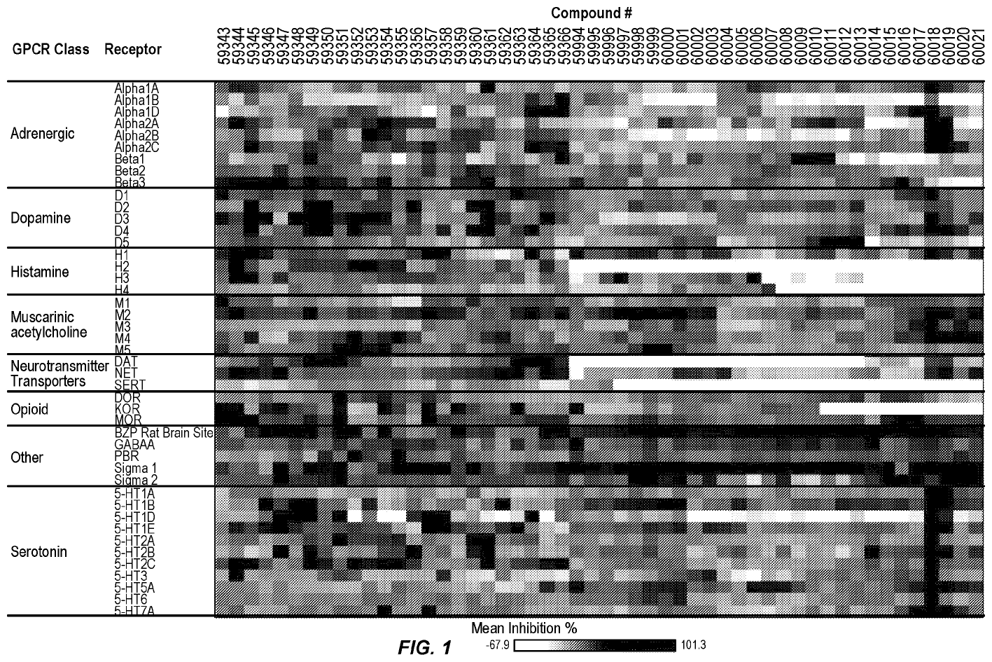

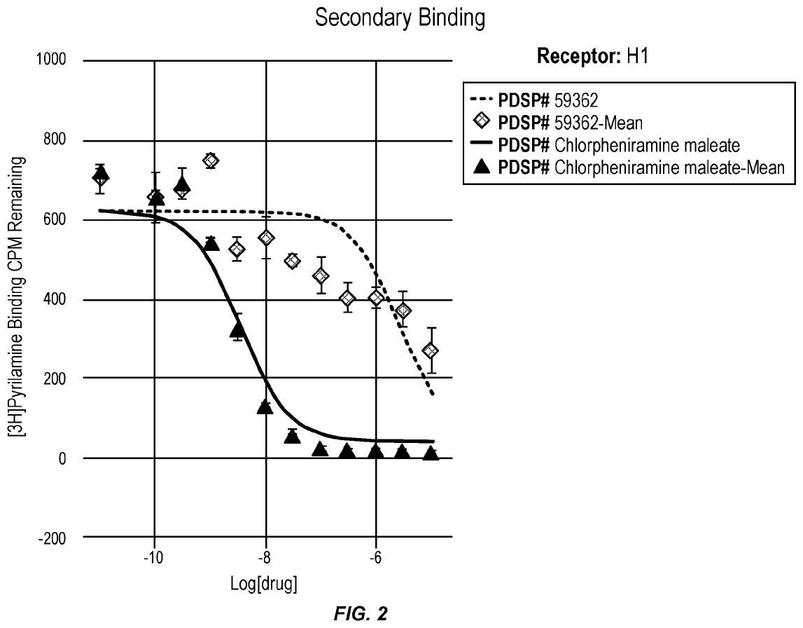

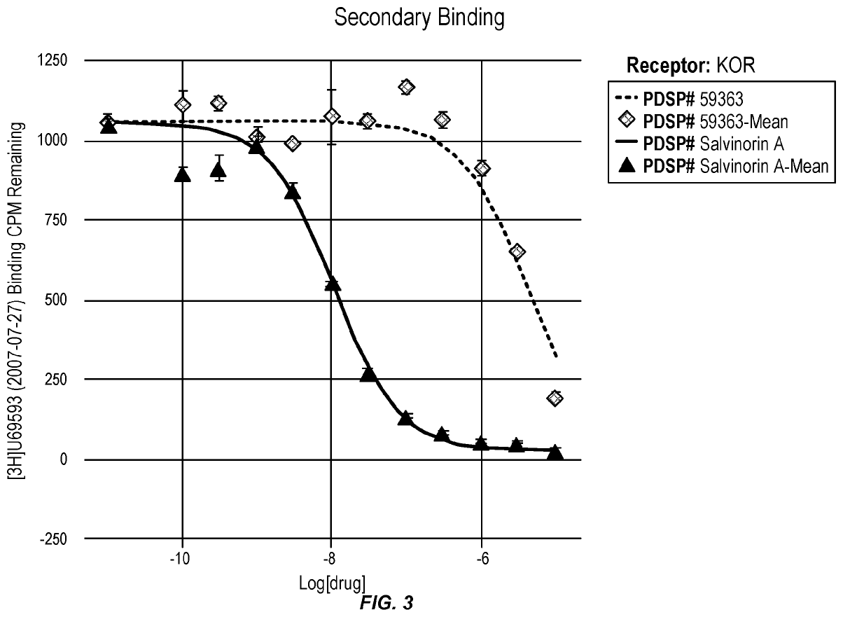

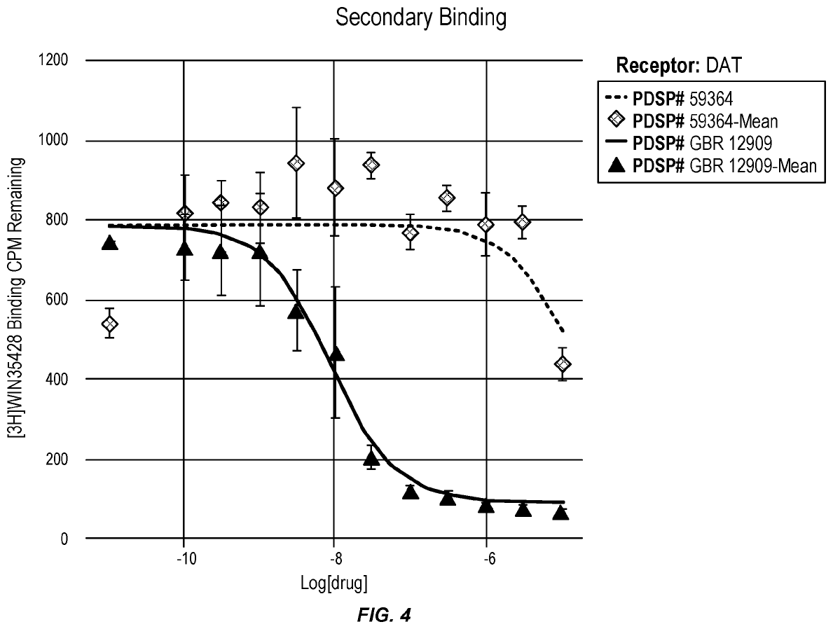

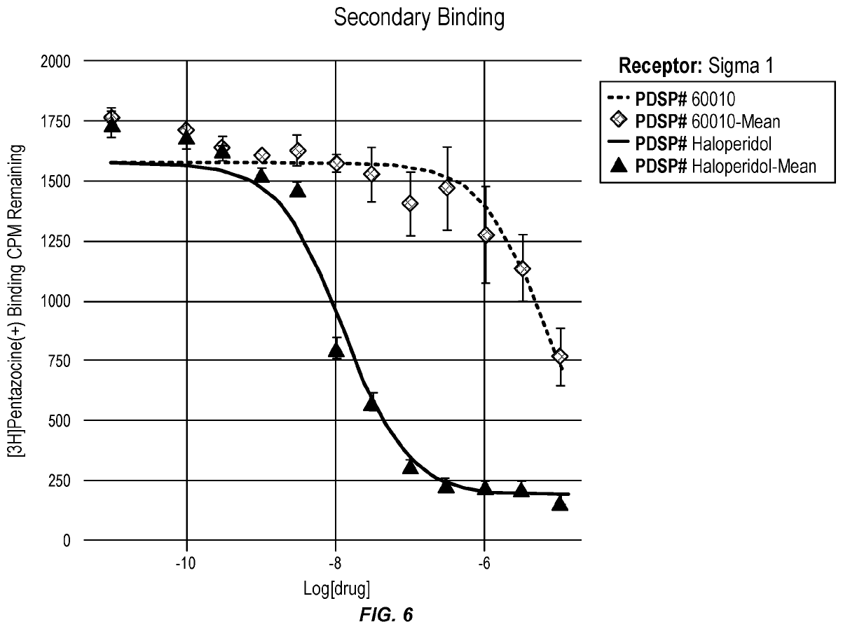

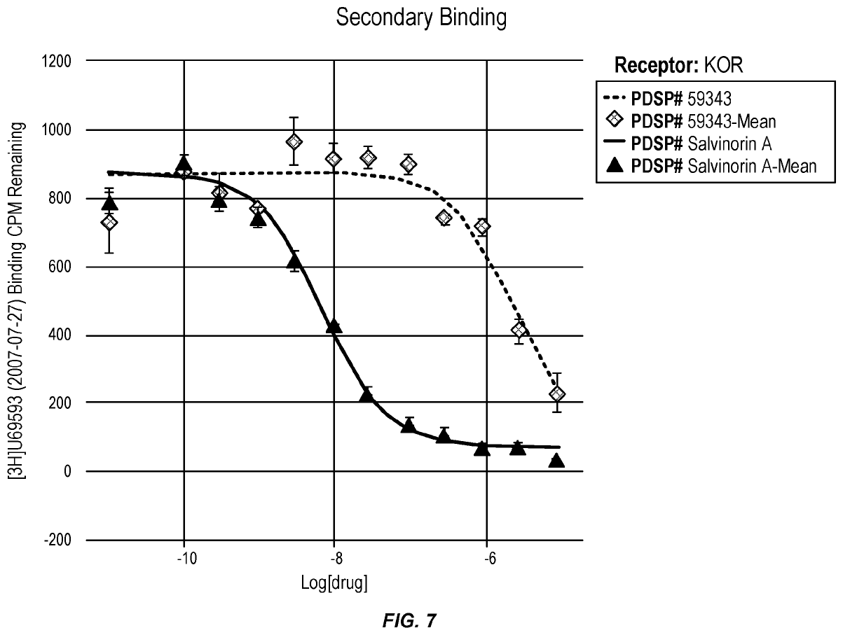

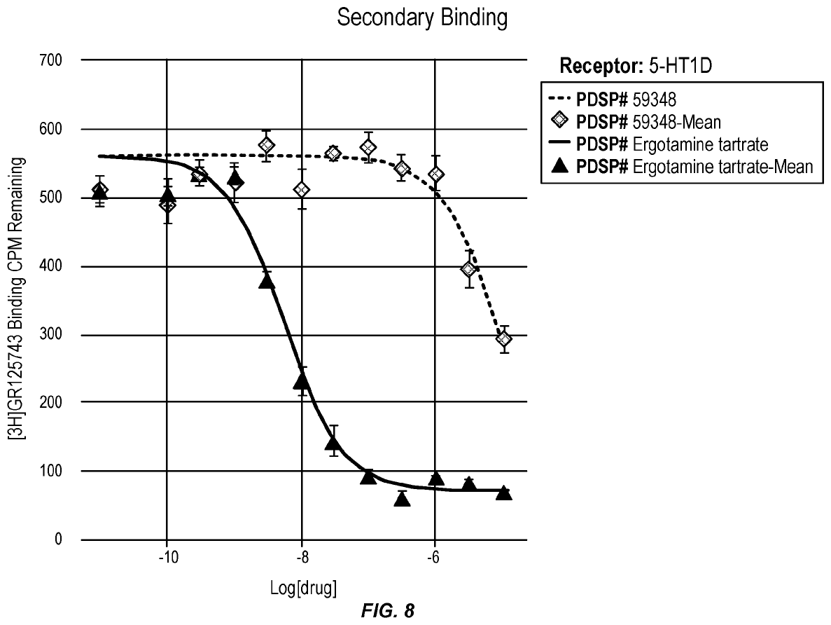

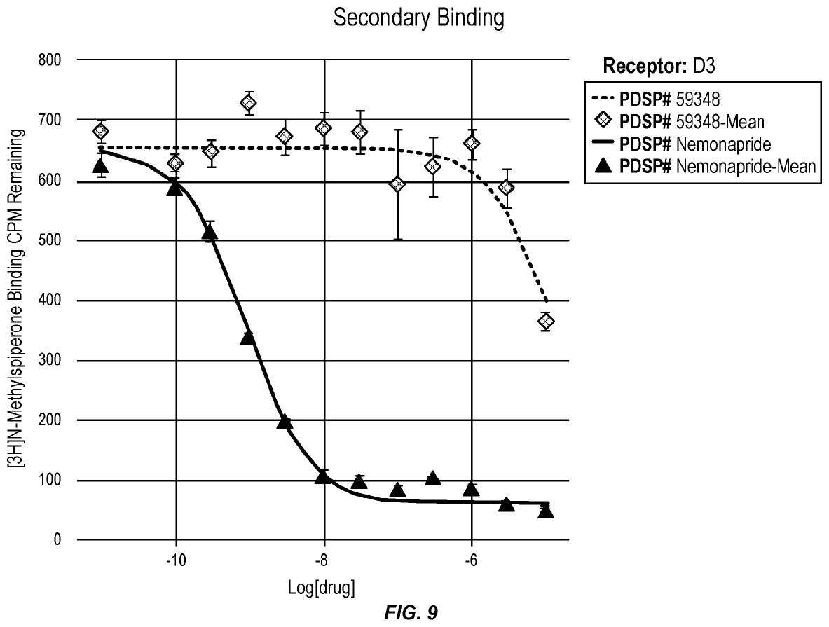

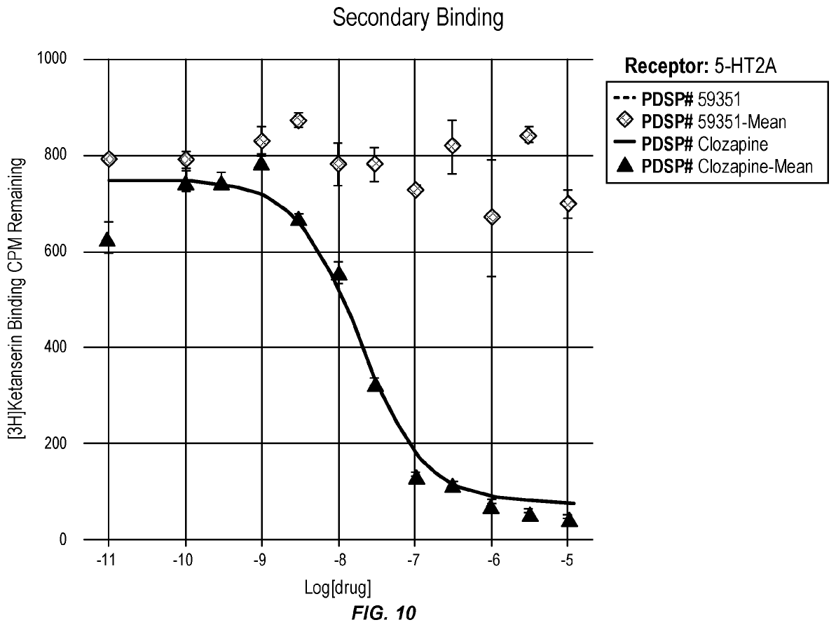

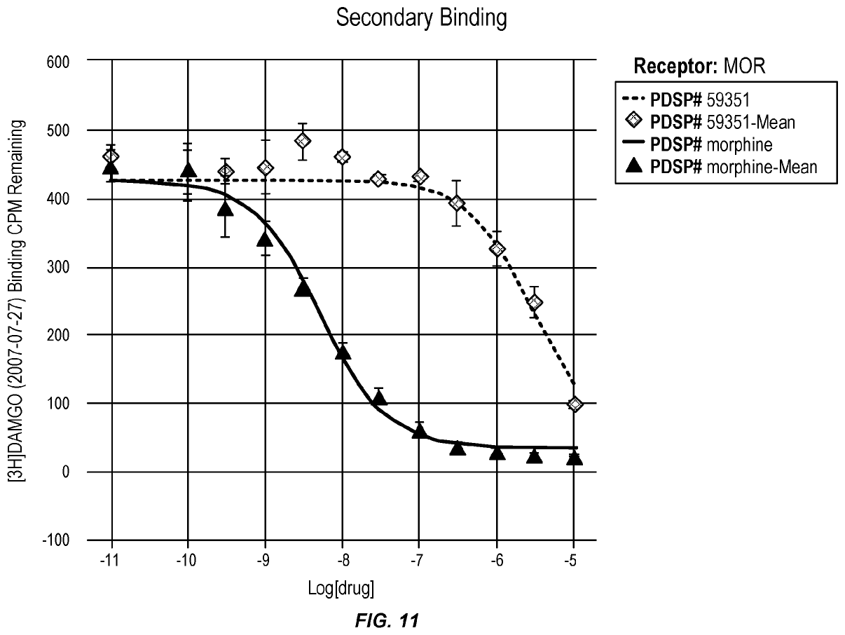

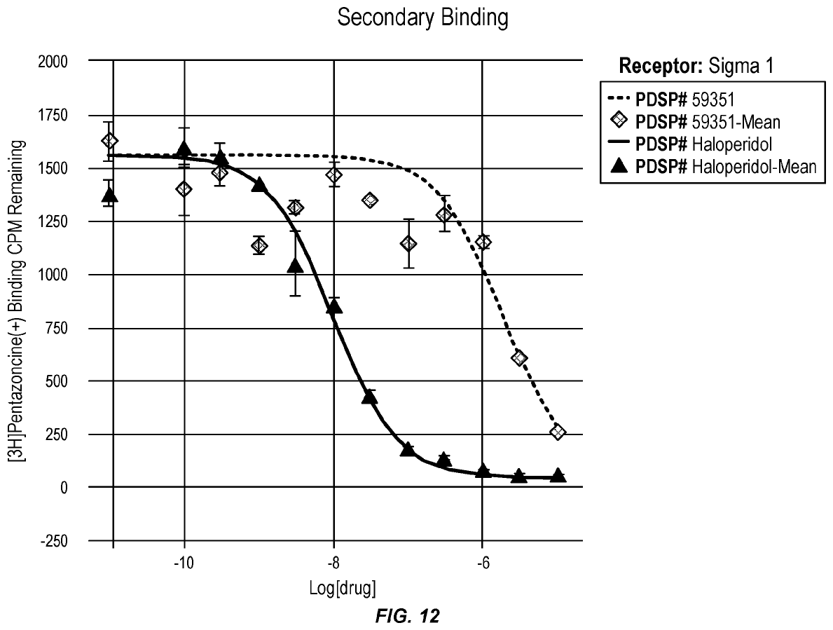

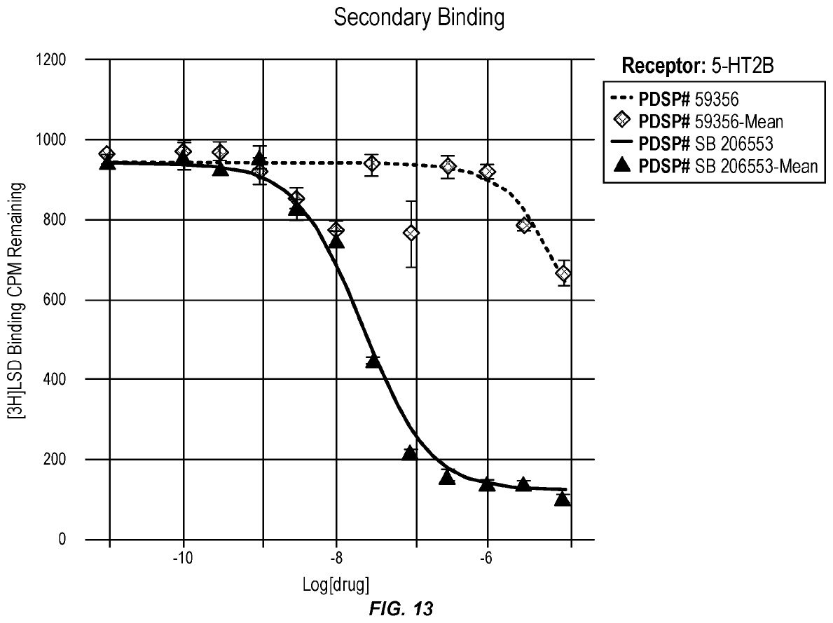

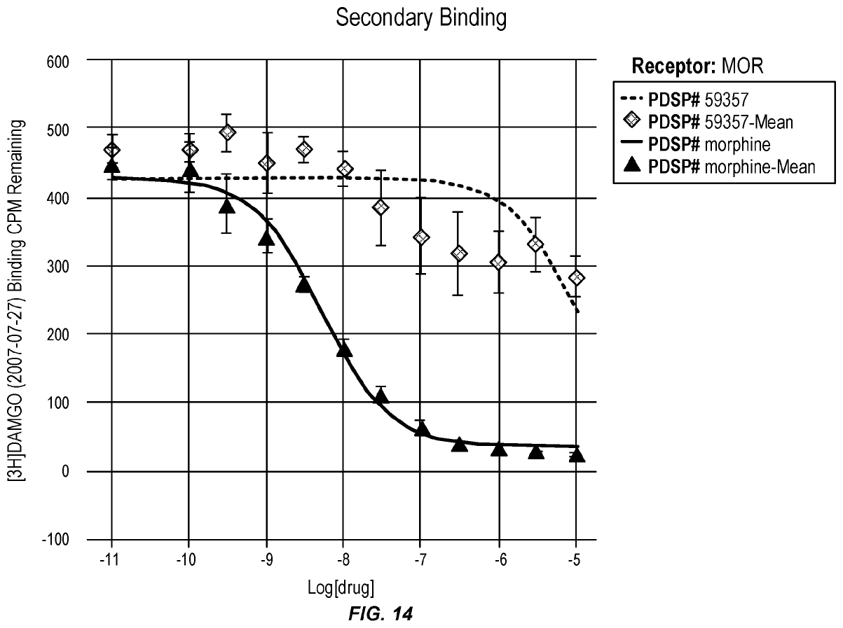

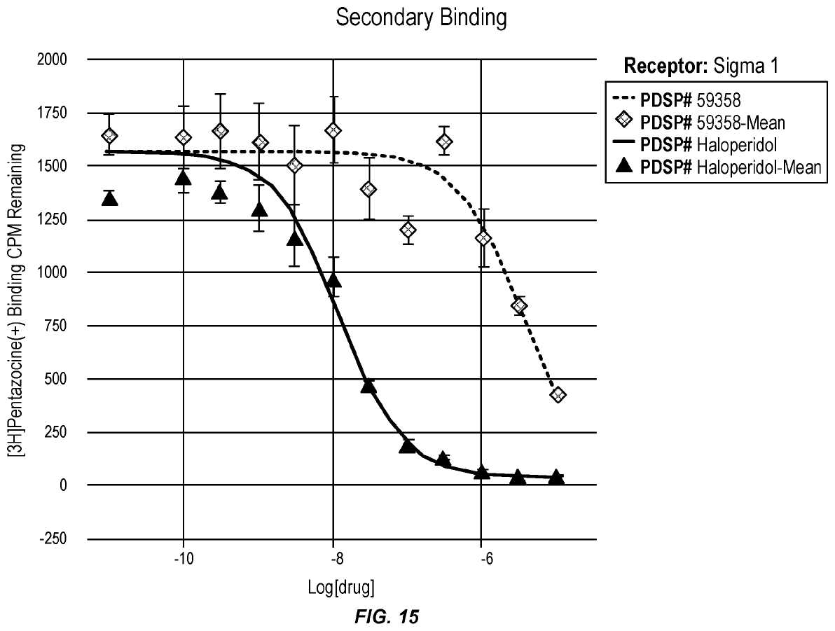

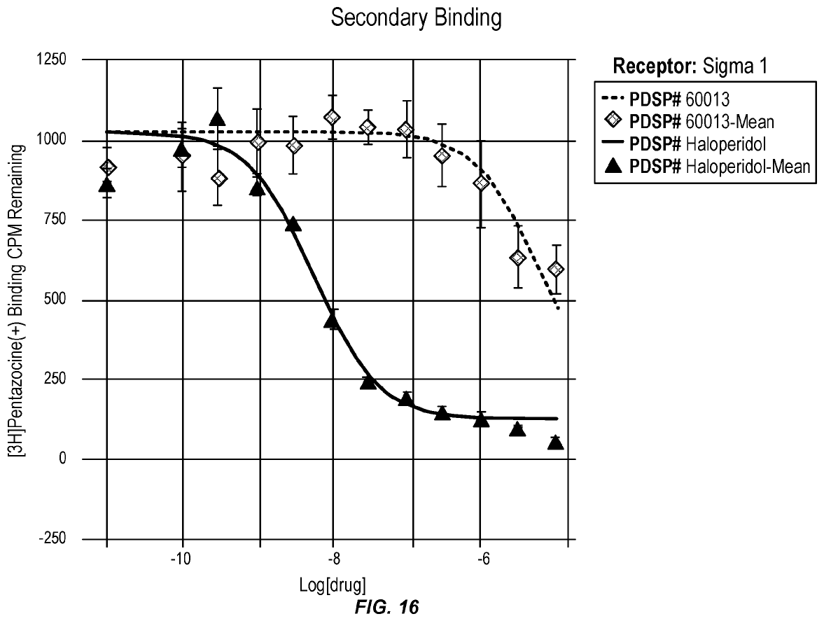

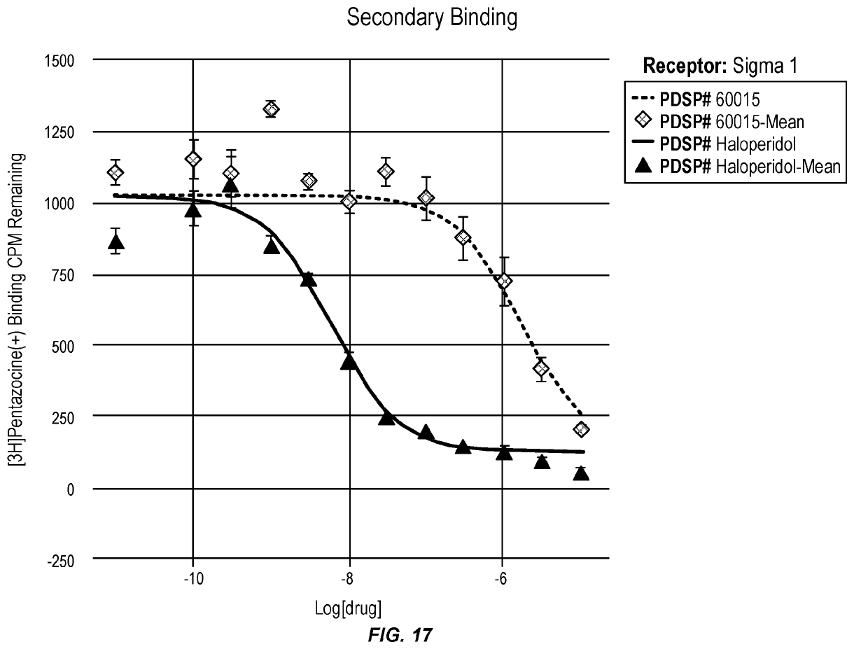

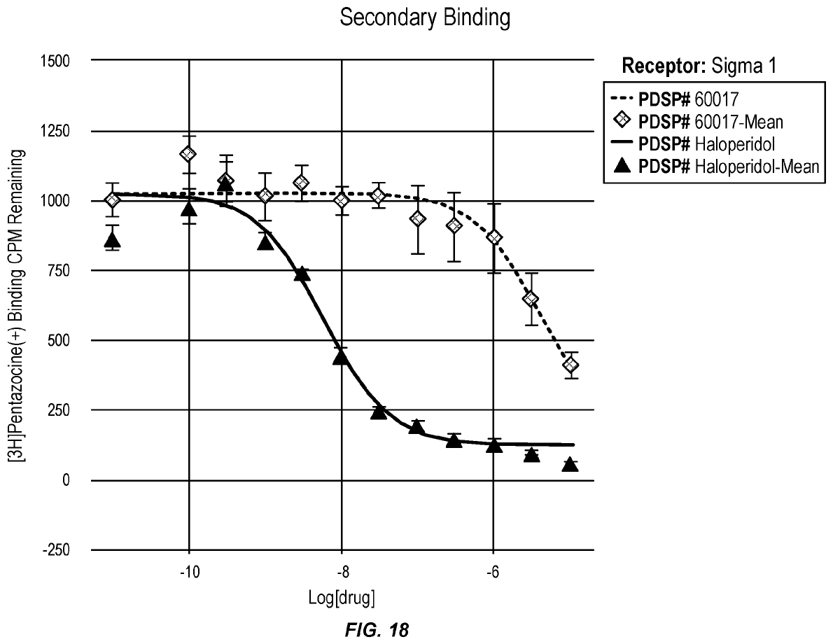

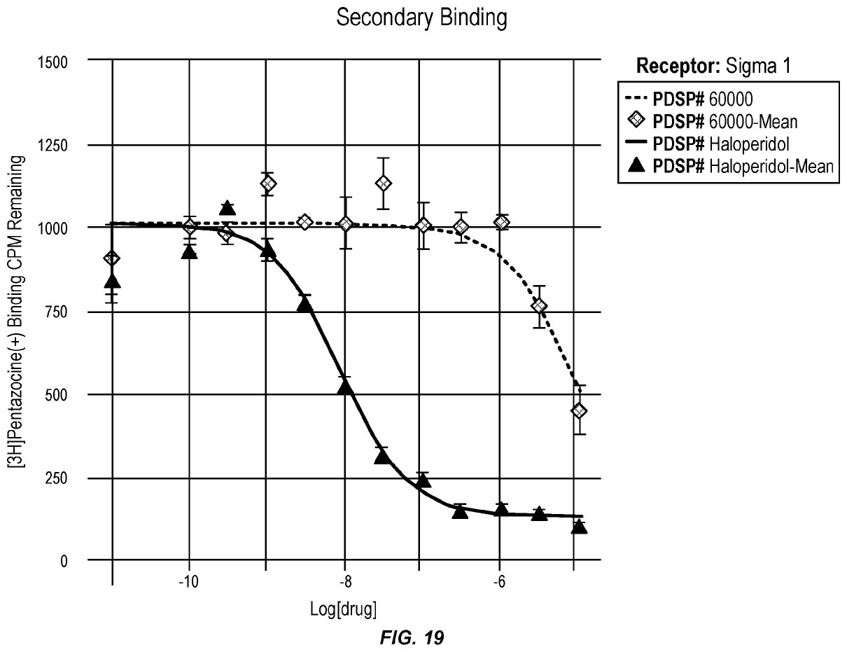

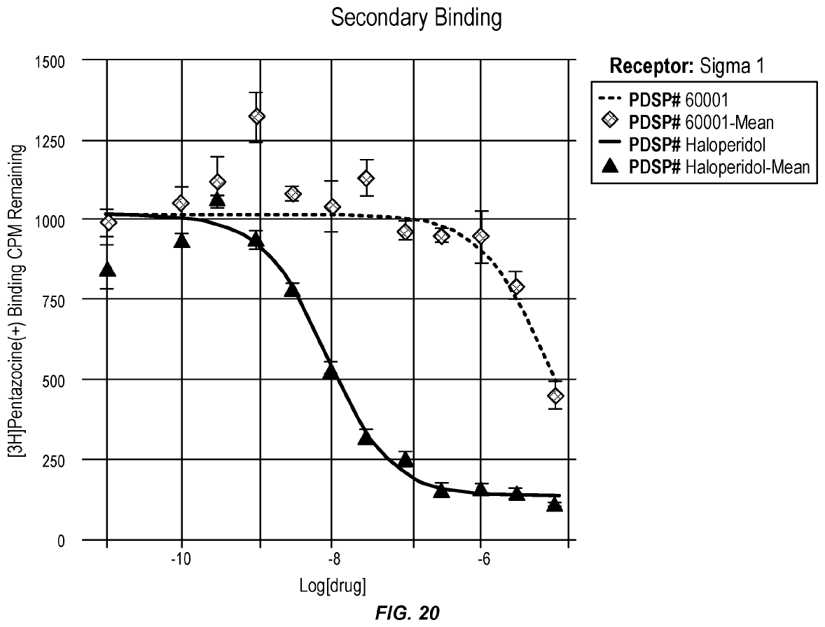

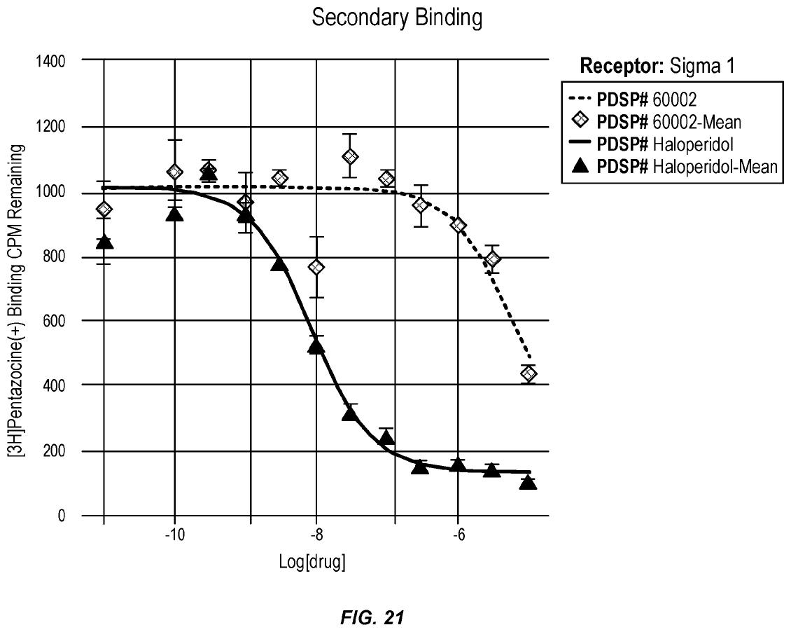

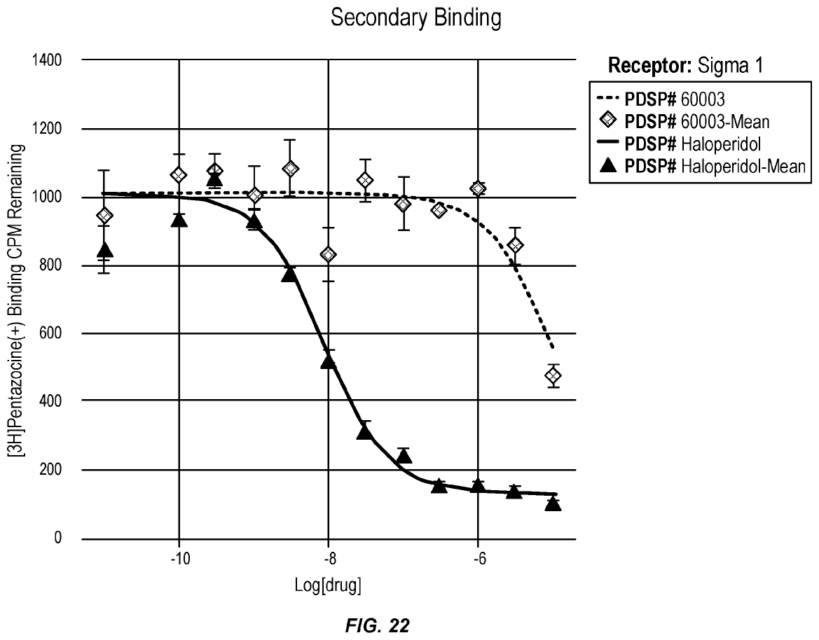

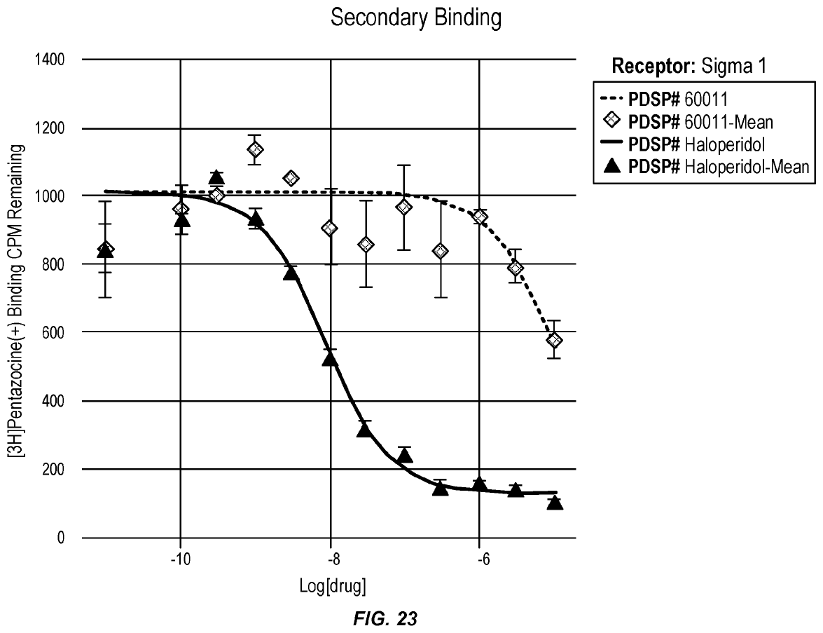

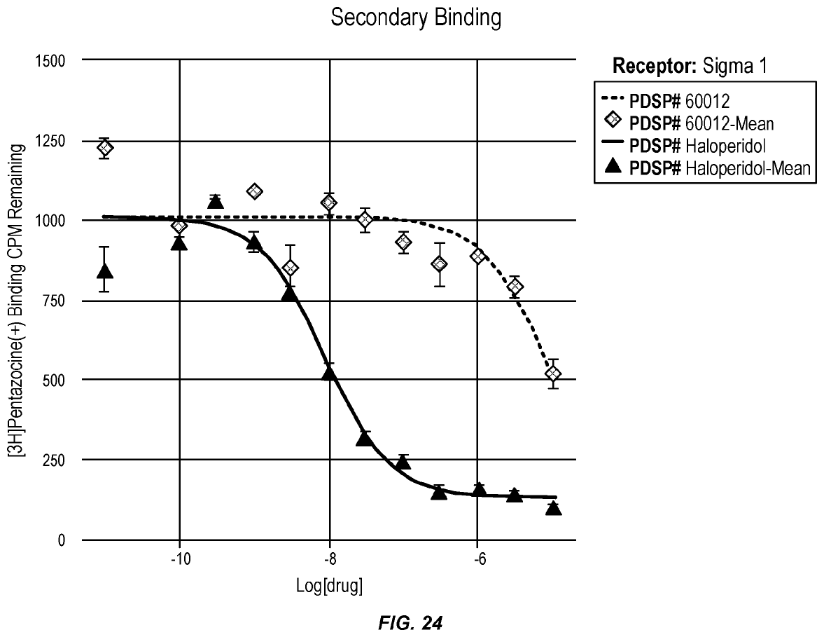

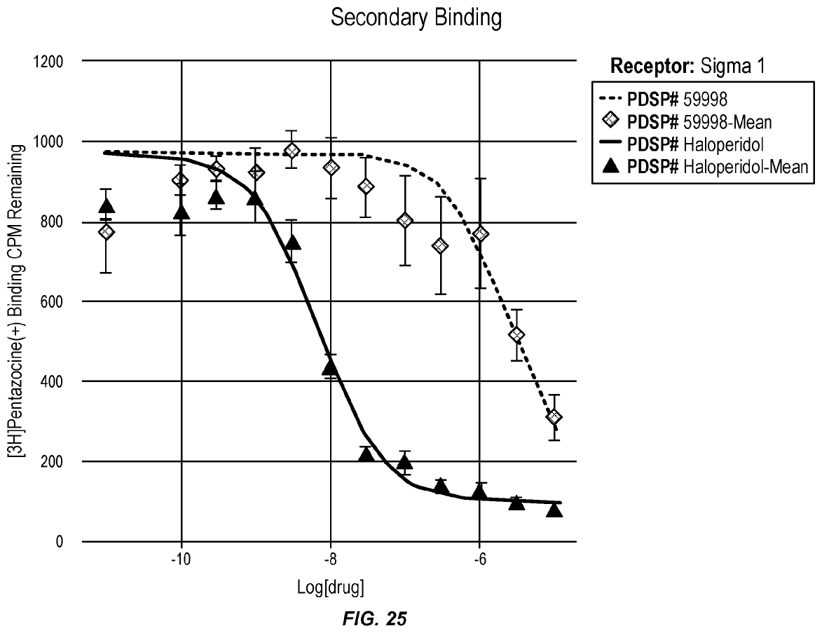

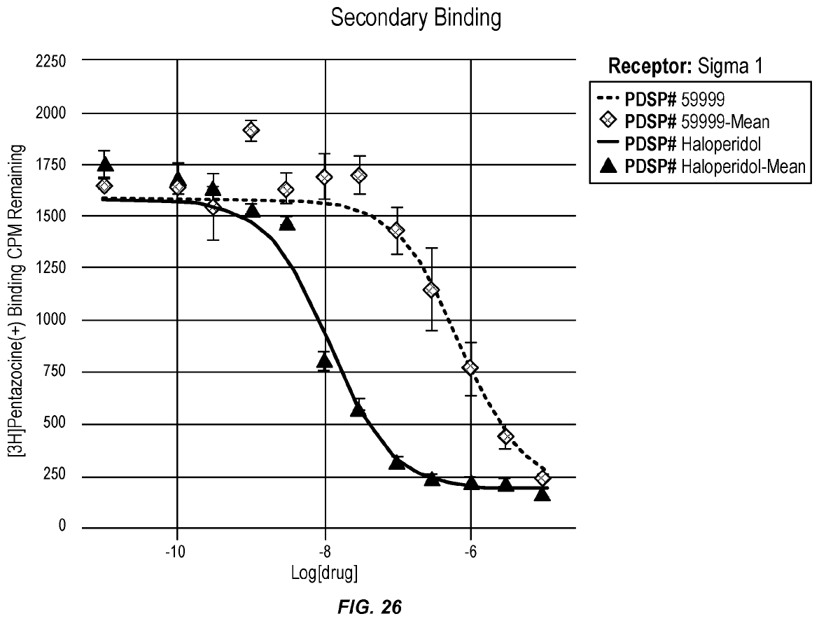

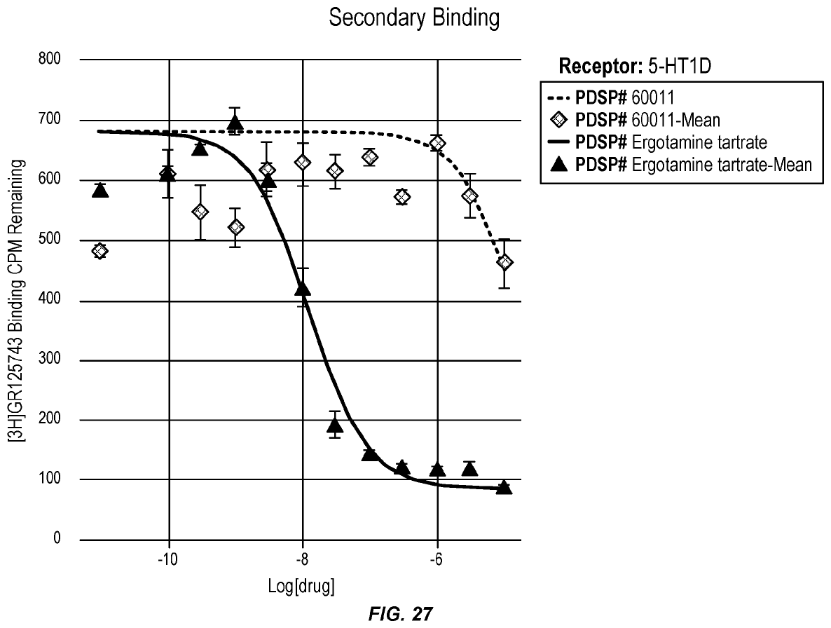

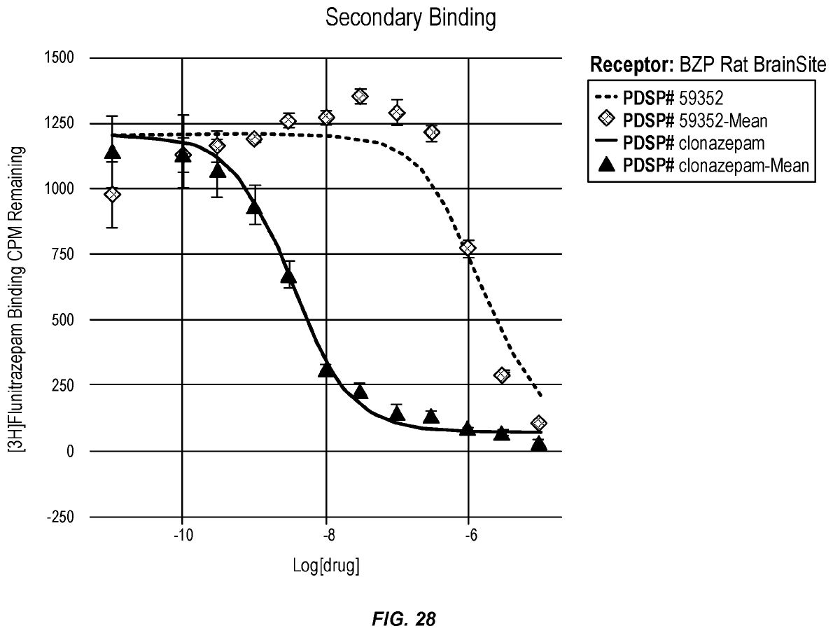

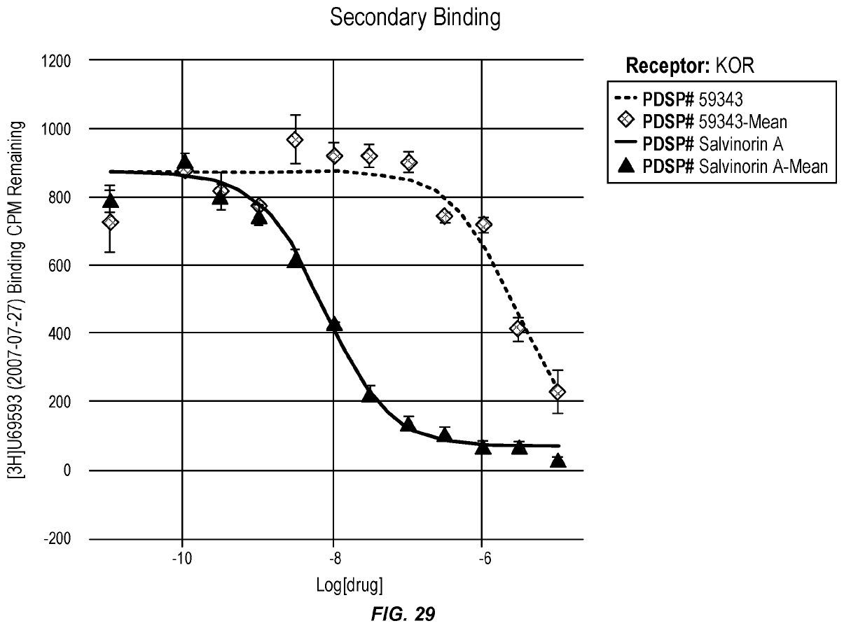

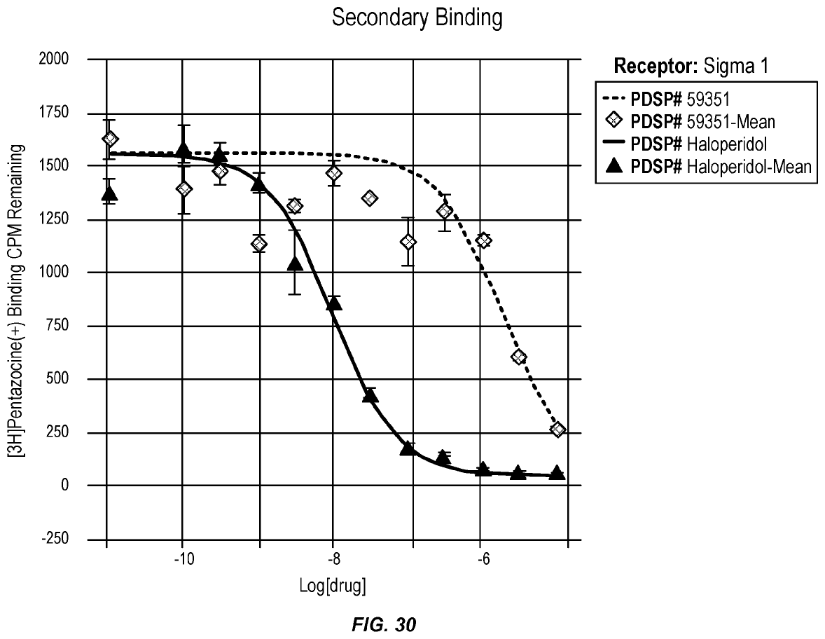

Results from a primary assay are shown in FIG. 1 and results from a secondary assay of the indicated compound against a certain GPCR are shown in FIGS. 2-30.

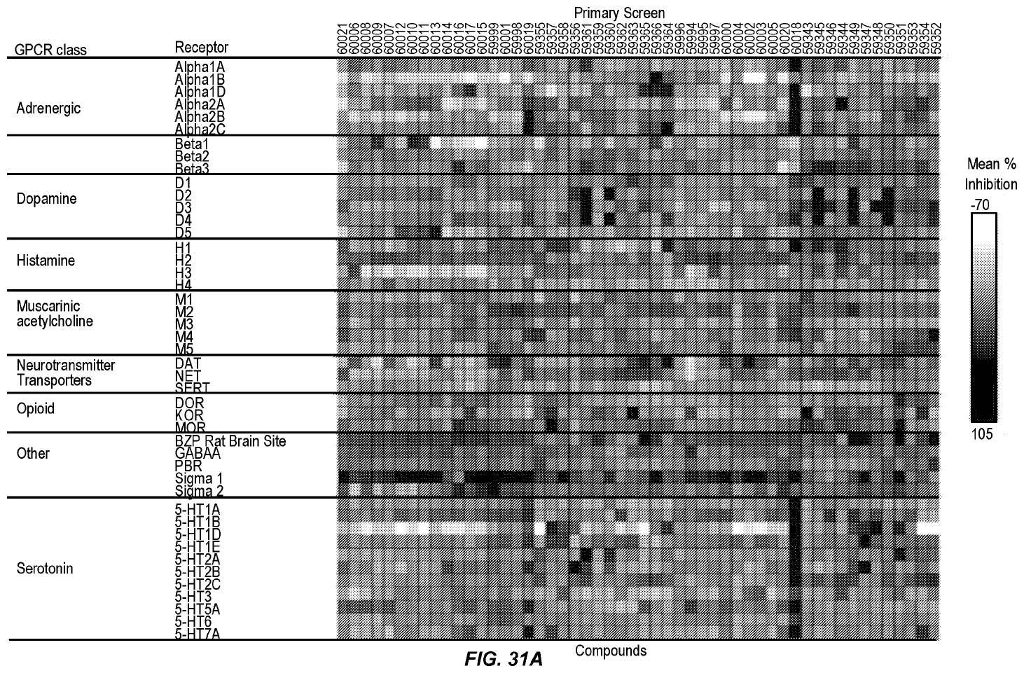

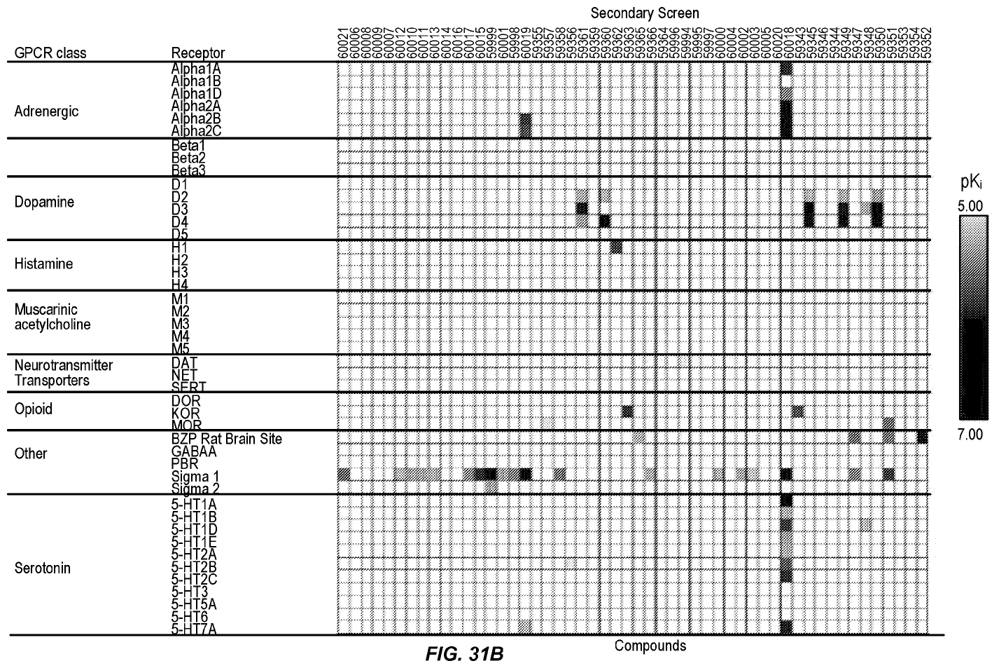

Additional polypharmacology profiles were assessed for compounds as described herein. See FIGS. 31A and 31B. The selected GPCR receptors and their associated class are shown in FIGS. 31A and 31B. The GPCR receptors included adrenergic receptors (Alpha-1A, Alpha-1B, Alpha-1D, Alpha-2A, Alpha-2B, Alpha-2C, Beta-1, Beta-2, and Beta-3); dopamine receptors (D1, D2, D3, D4, and D5); histamine receptors (H1, H2, H3, and H4); muscarinic acetylcholine receptors (M1, M2, M3, M4, and M5); neurotransmiltter transporters (DAT, NET, and SERT); opioid receptors (DOR, KOR, and MOR); serotonin receptors (5-HT1A, 5-HT1B, 5-HT1D, 5-HT1E, 5-HT2A, 5-HT2B, 5-HT2C, 5-HT3, 5-HT5A, 5-HT6, and 5-HT7A); and other receptors, including BZP rat brain site, GABAA, PBR, Sigma 1, and Sigma 2). The primary screen was carried out at a compound concentration of 10 μM and the mean % inhibition was determined. See FIG. 31A for results. For compounds exhibiting greater than 50% inhibition, a secondary dose-response screen was performed. See FIG. 31B for results.

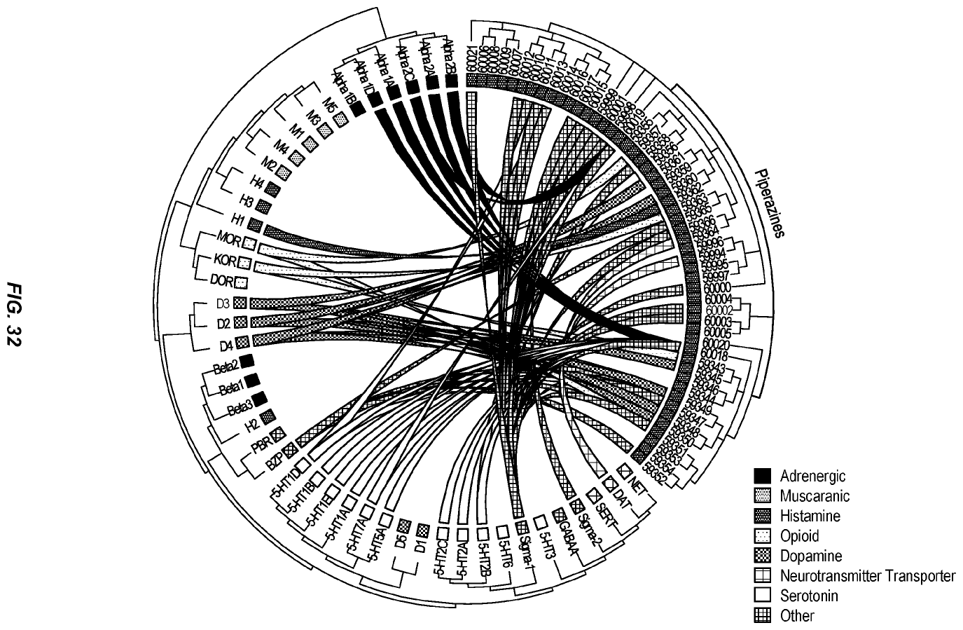

A GPCR chord analysis was also performed to show the activity of the compounds described herein versus the GPCRs noted above in a dose-response screen, and the results are shown in the chord plot in FIG. 32.

The chord plot was created from secondary screening data (for compounds with pKi<5) using Circos (see Krzywinski et al., Genome Res., 19:1639-1645 (2009). The dendrogram for 45 GPCRs was created using MEGA11 (see Tamura et al., Molecular Biology and Evolution, 38(7):3022-3027 (2021)). Protein sequences were obtained from UniProt (see The UniProt Consortium, Nucleic Acids Research, 49(D1): D480-D489 (2021)). Initial trees for the heuristic search were obtained automatically by applying Neighbor-Join and BioNJ algorithms to a matrix of pairwise distances estimated using the Maximum Likelihood method and JTT matrix-based model (see Jones et al., Bioinformatics, 8(3): 275-282 (1992)), and then selecting the tree with the highest log likelihood. The dendrogram for the SDFL was created manually to reflect the structural relationships of the compounds across different levels, namely enantiomers, diastereomers, regioisomers, N1 substituent, and N4 substituent.

The GPCRs are presented in FIG. 32 with a phylogenetic tree. The compounds described herein are clustered hierarchically based on structural features.

The compounds and methods of the appended claims are not limited in scope by the specific compounds and methods described herein, which are intended as illustrations of a few aspects of the claims and any compounds and methods that are functionally equivalent are within the scope of this disclosure. Various modifications of the compounds and methods in addition to those shown and described herein are intended to fall within the scope of the appended claims. Further, while only certain representative compounds, methods, and aspects of these compounds and methods are specifically described, other compounds and methods are intended to fall within the scope of the appended claims. Thus, a combination of steps, elements, components, or constituents can be explicitly mentioned herein; however, all other combinations of steps, elements, components, and constituents are included, even though not explicitly stated.

1. A compound of the following formula: 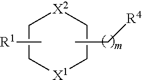
or a pharmaceutically acceptable salt or prodrug thereof, wherein:
m is 0 or 1;
X¹ is NR², ⁺NR²R², or O;
X² is NR³, ⁺NR³R³, or O;
R¹ is substituted or unsubstituted alkyl, substituted or unsubstituted heteroalkyl, substituted or unsubstituted aryl, substituted or unsubstituted heteroaryl;
each R² and R³ are independently selected from the group consisting of hydrogen, substituted or unsubstituted alkyl, substituted or unsubstituted carbonyl, substituted or unsubstituted sulfonyl, substituted or unsubstituted aryl, or substituted or unsubstituted heteroaryl; and
R⁴ is substituted or unsubstituted alkyl, substituted or unsubstituted amino, hydroxyl, substituted or unsubstituted alkoxy, substituted or unsubstituted carbonyl, or substituted or unsubstituted sulfonyl,
wherein X¹ and X² are not simultaneously O; and
wherein two of R¹, R², R³, and R⁴, when present and adjacent, can combine to form a substituted or unsubstituted heterocycloalkyl or a substituted or unsubstituted heteroaryl.

2. The compound of claim 1, having the following formula: 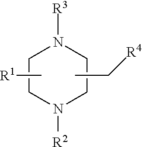
or a pharmaceutically acceptable salt or prodrug thereof, wherein:
R¹ is substituted or unsubstituted alkyl, substituted or unsubstituted heteroalkyl, substituted or unsubstituted aryl, substituted or unsubstituted heteroaryl;
R² and R³ are each independently selected from the group consisting of hydrogen, substituted or unsubstituted alkyl, substituted or unsubstituted carbonyl, substituted or unsubstituted sulfonyl, substituted or unsubstituted aryl, or substituted or unsubstituted heteroaryl; and
R⁴ is substituted or unsubstituted alkyl, substituted or unsubstituted amino, hydroxyl, substituted or unsubstituted alkoxy, substituted or unsubstituted carbonyl, or substituted or unsubstituted sulfonyl,
wherein R¹ and R³, R¹ and R², R² and R⁴, or R³ and R⁴ can combine to form a substituted or unsubstituted heterocycloalkyl or a substituted or unsubstituted heteroaryl.

3. The compound of claim 1, wherein R¹ is a side chain of an amino acid, wherein the amino acid is a natural amino acid or an unnatural amino acid.

4. (canceled)

5. (canceled)

6. The compound of claim 1, wherein R¹ is methyl.

7. The compound of claim 1, wherein R² and R³ are each independently selected from the group consisting of hydrogen, methyl, acetyl, —SO₂CH₃, phenyl, benzyl, benzoyl, aryl-substituted sulfonyl, heteroaryl-substituted sulfonyl, formyl, —CO₂R⁶, and —CONHR⁶,
wherein R⁶ is hydrogen, substituted or unsubstituted alkyl, substituted or unsubstituted heteroalkyl, substituted or unsubstituted aryl, or substituted or unsubstituted heteroaryl.

8. The compound of claim 1, wherein R⁴ is —CO₂R⁵, wherein R⁵ is hydrogen, substituted or unsubstituted alkyl, substituted or unsubstituted heteroalkyl, substituted or unsubstituted aryl, or substituted or unsubstituted heteroaryl.

9. (canceled)

10. The compound of claim 1, wherein the compound is selected from the group consisting of: 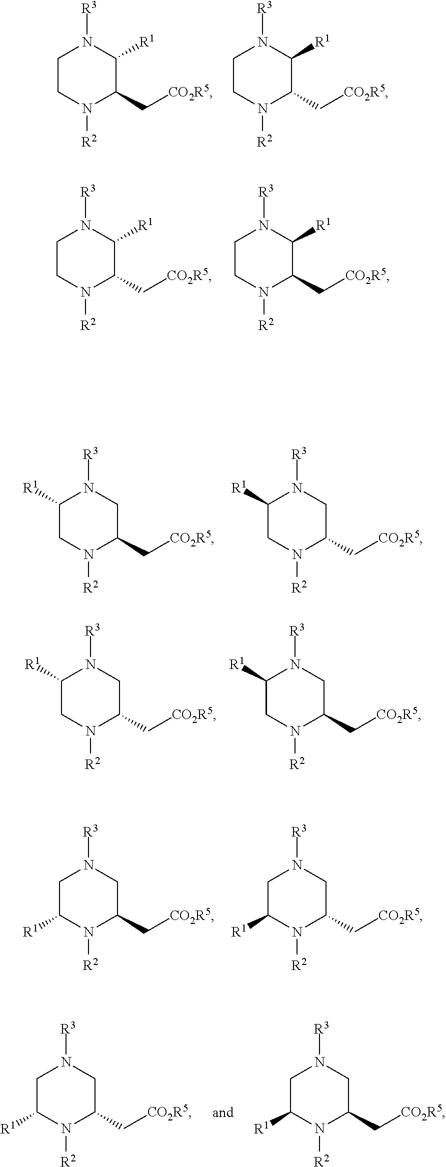
wherein
R⁵ is hydrogen, substituted or unsubstituted alkyl, substituted or unsubstituted heteroalkyl, substituted or unsubstituted aryl, or substituted or unsubstituted heteroaryl.

11.-22. (canceled)

23. The compound of claim 1, wherein the compound has the following formula: 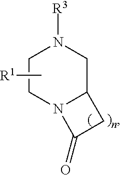
wherein n is 1, 2, or 3, or
wherein the compound has the following formula: 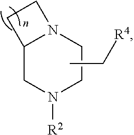
wherein n is 0, 1, 2, or 3.

24. The compound of claim 1, wherein the compound has the following formula: 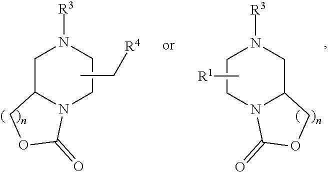
wherein n is 1 or 2.

25. A compound of the following formula: 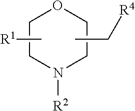
or a pharmaceutically acceptable salt or prodrug thereof, wherein:
R¹ is substituted or unsubstituted alkyl, substituted or unsubstituted heteroalkyl, substituted or unsubstituted aryl, substituted or unsubstituted heteroaryl;
R² is hydrogen, substituted or unsubstituted alkyl, substituted or unsubstituted carbonyl, substituted or unsubstituted sulfonyl, substituted or unsubstituted aryl, or substituted or unsubstituted heteroaryl; and
R⁴ is substituted or unsubstituted alkyl, substituted or unsubstituted amino, hydroxyl, substituted or unsubstituted alkoxy, substituted or unsubstituted carbonyl, or substituted or unsubstituted sulfonyl,
wherein R¹ and R² or R² and R⁴ can combine to form a substituted or unsubstituted heterocycloalkyl or a substituted or unsubstituted heteroaryl.

26. A compound of the following formula: 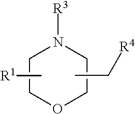
or a pharmaceutically acceptable salt or prodrug thereof, wherein:
R¹ is substituted or unsubstituted alkyl, substituted or unsubstituted heteroalkyl, substituted or unsubstituted aryl, substituted or unsubstituted heteroaryl;
R³ is hydrogen, substituted or unsubstituted alkyl, substituted or unsubstituted carbonyl, substituted or unsubstituted sulfonyl, substituted or unsubstituted aryl, or substituted or unsubstituted heteroaryl; and
R⁴ is substituted or unsubstituted alkyl, substituted or unsubstituted amino, hydroxyl, substituted or unsubstituted alkoxy, substituted or unsubstituted carbonyl, or substituted or unsubstituted sulfonyl,
wherein R¹ and R³ or R³ and R⁴ can combine to form a substituted or unsubstituted heterocycloalkyl or a substituted or unsubstituted heteroaryl.

27. A compound of the following formula: 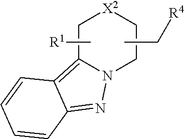
or a pharmaceutically acceptable salt or prodrug thereof, wherein:
X² is NR² or O;
R¹ is substituted or unsubstituted alkyl, substituted or unsubstituted heteroalkyl, substituted or unsubstituted aryl, or substituted or unsubstituted heteroaryl;
R⁴ is substituted or unsubstituted alkyl, substituted or unsubstituted amino, hydroxyl, substituted or unsubstituted alkoxy, substituted or unsubstituted carbonyl, or substituted or unsubstituted sulfonyl.

28. The compound of claim 27, wherein the compound has the following formula:

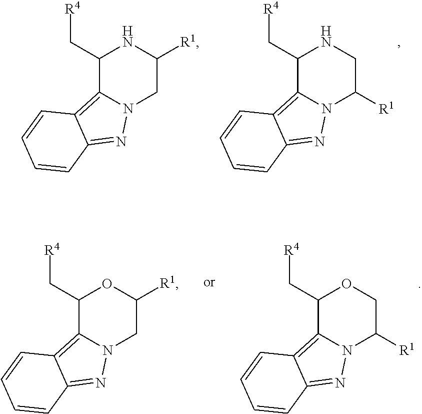

29. A compound of the following formula: 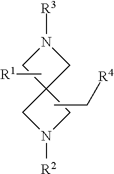
or a pharmaceutically acceptable salt or prodrug thereof, wherein:
R¹ is substituted or unsubstituted alkyl, substituted or unsubstituted heteroalkyl, substituted or unsubstituted aryl, or substituted or unsubstituted heteroaryl;
R² and R³ are independently selected from the group consisting of hydrogen, substituted or unsubstituted alkyl, substituted or unsubstituted carbonyl, substituted or unsubstituted sulfonyl, substituted or unsubstituted aryl, or substituted or unsubstituted heteroaryl; and
R⁴ is substituted or unsubstituted alkyl, substituted or unsubstituted amino, hydroxyl, substituted or unsubstituted alkoxy, substituted or unsubstituted carbonyl, or substituted or unsubstituted sulfonyl.

30. A pharmaceutical composition comprising a compound of claim 1 and a pharmaceutically acceptable carrier.

31. (canceled)

32. A method of treating or preventing a condition or disease associated with a G protein-coupled receptor in a subject, comprising administering to the subject an effective amount of a compound of claim 1.

33. The method of claim 32, wherein the condition or disease associated with a G protein-coupled receptor comprises ageing, cancer, a cardiovascular disorder, a hematologic disorder, an infectious disease, an inflammatory disease, a metabolic disease, a neurodegenerative disorder, a respiratory disease, or a urological disorder.

34. The method of claim 32, further comprising administering to the subject a second compound, biomolecule, or composition.

35. A method of regulating a G protein-coupled receptor in a cell, comprising contacting a cell with an effective amount of a compound of claim 1.

36. The method of claim 35, wherein the G protein-coupled receptor is a serotonin receptor, a dopamine receptor, a muscarinic receptor, an adrenergic receptor, a histamine receptor, or a sigma receptor.

37. The method of claim 35, wherein:
the G protein-coupled receptor is a serotonin receptor selected from the group consisting of 5-HT1A, 5-HT1B, 5-HT1D, 5-HT1E, 5-HT2A, 5-HT2B, 5-HT2C, 5-HT3, 5-HT5A, 5-HT6, and 5-HT7A;
the G protein-coupled receptor is a dopamine receptor selected from the group consisting of D1, D2, D3, D4, and D5;
the G protein-coupled receptor is a muscarinic receptor selected from the group consisting of M1, M2, M3, M4, and M5;
the G protein-coupled receptor is an adrenergic receptor selected from the group consisting of Alpha-1A, Alpha-1B, Alpha-1C, Alpha-2A, Alpha-2B, Alpha-2C, Beta-1, Beta-2, and Beta-3;
the G protein-coupled receptor is a histamine receptor selected from the group consisting of H1, H2, H3, and H4; or
the G protein-coupled receptor is a sigma receptor selected from the group consisting of Sigma-1 and Sigma-2.

38.-42. (canceled)

43. The method of claim 35, wherein the G protein-coupled receptor is selected from the group consisting of kappa-opioid receptor (KOR), mu-opioid receptor (MOR), delta-opioid receptor (DOR), gamma-aminobutyric acid A (GABAA) receptor, peripheral-type benzodiazepine receptors (PBR), serotonin transporter (SERT) receptor, and benzodiazepine (BZP) rat brain receptors.

44. (canceled)
